SEQUENCE LISTING (12 sequences)

SEQ ID NO: 1 - DNA, 2056 bp, Mus musculus
cgaattcctcgcgccgtgggaggggcggcccggcccacccccacgccccgcccgggagggacggggggagagagagcgcggcgacgggtatctgggtcaaagctgttcggagaaattggaactacagttttatctagccacatctctgagaattctgaagaaagcagcaggtgaaagtcattgccaagtgattttgttctgtaaggaagcctccctcattcacttacaccagtgagacagcaggaccagtcattcaaagggccgtgtacaggacgcgtgcgaatcagacaatgactcagctatacacttacatcagattactgggagcctgtctgttcatcatttctcatgttcaagggcagaatctagatagtatgctccatggcactggtatgaaatcagacttggaccagaagaagccagaaaatggagtgactttagcaccagaggataccttgcctttcttaaagtgctattgctcaggacactgcccagatgatgctattaataacacatgcataactaatggccattgctttgccattatagaagaagatgatcagggagaaaccacattaacttctgggtgtatgaagtatgaaggctctgattttcaatgcaaggattcaccgaaagcccagctacgcaggacaatagaatgttgtcggaccaatttgtgcaaccagtatttgcagcctacactgccccctgttgttataggtccgttctttgatggcagcatccgatggctggttgtgctcatttccatggctgtctgtatagttgctatgatcatcttctccagctgcttttgctataagcattattgtaagagtatctcaagcaggggtcgttacaaccgtgatttggaacaggatgaagcatttattccagtaggagaatcattgaaagacctgattgaccagtcccaaagctctgggagtggatctggattgcctttattggttcagcgaactattgccaaacagattcagatggttcggcaggttggtaaaggccgctatggagaagtatggatgggtaaatggcgtggtgaaaaagtggctgtcaaagtgttttttaccactgaagaagctagctggtttagagaaacagaaatctaccagacggtgttaatgcgtcatgaaaatatacttggttttatagctgcagacattaaaggcactggttcctggactcagctgtatttgattactgattaccatgaaaatggatctctctatgacttcctgaaatgtgccacactagacaccagagccctactcaagttagcttattctgctgcttgtggtctgtgccacctccacacagaaatttatggtacccaagggaagcctgcaattgctcatcgagacctgaagagcaaaaacatccttattaagaaaaatggaagttgctgtattgctgacctgggcctagctgttaaattcaacagtgatacaaatgaagttgacatacccttgaataccagggtgggcaccaagcggtacatggctccagaagtgctggatgaaagcctgaataaaaaccatttccagccctacatcatggctgacatctatagctttggtttgatcatttgggaaatggctcgtcgttgtattacaggaggaatcgtggaggaatatcaattaccatattacaacatggtgcccagtgacccatcctatgaggacatgcgtgaggttgtgtgtgtgaaacgcttgcggccaatcgtgtctaaccgctggaacagcgatgaagtaagttggagccaagtccctgtaaagtgatgagtgagtgccgagttactctgtgctcaccacactctgtttgcatttatttctctttagtgtcttcgagcagttttgaagctaatgtcagaatgttgggcccataatccagcctccagactcacagctttgagaatcaagaagacacttgcaaaaatggttgaatcccaggatgtaaagatttgacaattaaacaattttgagggagaatttagactgcaagaacttcttcacccaaggaaggaattcctgcaggcccgggg

SEQ ID NO: 2 - DNA, 1891 bp, Mus musculus
cgaattcctcgcgccgtgggaggggcggcccggcccacccccacgccccgcccgggagggacggggggagagagagcgcggcgacgggtatctgggtcaaagctgttcggagaaattggaactacagttttatctagccacatctctgagaattctgaagaaagcagcaggtgaaagtcattgccaagtgattttgttctgtaaggaagcctccctcattcacttacaccagtgagacagcaggaccagtcattcaaagggccgtgtacaggacgcgtgcgaatcagacaatgactcagctatacacttacatcagattactgggagcctgtctgttcatcatttctcatgttcaaactaatggccattgctttgccattatagaagaagatgatcagggagaaaccacattaacttctgggtgtatgaagtatgaaggctctgattttcaatgcaaggattcaccgaaagcccagctacgcaggacaatagaatgttgtcggaccaatttgtgcaaccagtatttgcagcctacactgccccctgttgttataggtccgttctttgatggcagcatccgatggctggttgtgctcatttccatggctgtctgtatagttgctatgatcatcttctccagctgcttttgctataagcattattgtaagagtatctcaagcaggggtcgttacaaccgtgatttggaacaggatgaagcatttattccagtaggagaatcattgaaagacctgattgaccagtcccaaagctctgggagtggatctggattgcctttattggttcagcgaactattgccaaacagattcagatggttcggcaggttggtaaaggccgctatggagaagtatggatgggtaaatggcgtggtgaaaaagtggctgtcaaagtgttttttaccactgaagaagctagctggtttagagaaacagaaatctaccagacggtgttaatgcgtcatgaaaatatacttggttttatagctgcagacattaaaggcactggttcctggactcagctgtatttgattactgattaccatgaaaatggatctctctatgacttcctgaaatgtgccacactagacaccagagccctactcaagttagcttattctgctgcttgtggtctgtgccacctccacacagaaatttatggtacccaagggaagcctgcaattgctcatcgagacctgaagagcaaaaacatccttattaagaaaaatggaagttgctgtattgctgacctgggcctagctgttaaattcaacagtgatacaaatgaagttgacatacccttgaataccagggtgggcaccaagcggtacatggctccagaagtgctggatgaaagcctgaataaaaaccatttccagccctacatcatggctgacatctatagctttggtttgatcatttgggaaatggctcgtcgttgtattacaggaggaatcgtggaggaatatcaattaccatattacaacatggtgcccagtgacccatcctatgaggacatgcgtgaggttgtgtgtgtgaaacgcttgcggccaatcgtgtctaaccgctggaacagcgatgaagtaagttggagccaagtccctgtaaagtgatgagtgagtgccgagttactctgtgctcaccacactctgtttgcatttatttctctttagtgtcttcgagcagttttgaagctaatgtcagaatgttgggcccataatccagcctccagactcacagctttgagaatcaagaagacacttgcaaaaatggttgaatcccaggatgtaaagatttgacaattaaacaattttgagggagaatttagactgcaagaacttcttcacccaaggaaggaattcctgcaggcccgggg

SEQ ID NO: 3 - DNA, 165 bp, Mus Musulus
gggcagaatctagatagtatgctccatggcactggtatgaaatcagacttggaccagaagaagccagaaaatggagtgactttagcaccagaggataccttgcctttcttaaagtgctattgctcaggacactgcccagatgatgctattaataacacatgcata

SEQ ID NO: 4 - protein, 500 aa, Mus musculus
MTQLYTYIRLLGACLFIISHVQGQNLDSMLHGTGMKSDLDQKKPENGVTLAPEDTLPFLKCYCSGHCPDDAINNTCITNGHCFAIIEEDDQGETTLTSGCMKYEGSDFQCKDSPKAQLRRTIECCRTNLCNQYLQPTLPPVVIGPFFDGSIRWLVVLISMAVCIVAMIIFSSCFCYKHYCKSISSRGRYNRDLEQDEAFIPVGESLKDLIDQSQSSGSGSGLPLLVQRTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDIPLNTRVGTKRYMAPEVLDESLNKNHFQPYIMADIYSFGLIIWEMARRCITGGIVEEYQLPYYNMVPSDPSYEDMREVVCVKRLRPIVSNRWNSDEVSWSQVPVK

SEQ ID NO: 5 - protein, 456 aa, Mus musculus
MTQLYTYIRLLGACLFIISHVQGQNLDSMLHGTTNGHCFAIIEEDDQGETTLTSGCMKYEGSDFQCKDSPKAQLRRTIECCRTNLCNQYLQPTLPPVVIGPFFDGSIRWLVVLISMAVCIVAMIIFSSCFCYKHYCKSISSRGRYNRDLEQDEAFIPVGESLKDLIDQSQSSGSGSGLPLLVQRTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDIPLNTRVGTKRYMAPEVLDESLNKNHFQPYIMADIYSFGLIIWEMARRCITGGIVEEYQLPYYNMVPSDPSYEDMREVVCVKRLRPIVSNRWNSDEVSWSQVPVK

SEQ ID NO: 6 - protein, 44 aa, Mus musculus
GMKSDLDQKKPENGVTLAPEDTLPFLKCYCSGHCPDDAINNTCI

SEQ ID NO: 7 - protein, 532 aa, Homo sapiens
MTQLYIYIRLLGAYLFIISRVQGQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRWLVLLISMAVCIIAMIIFSSCFCYKHYCKSISSRRRYNRDLEQDEAFIPVGESLKDLIDQSQSSGSGSGLPLLVQRTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDVPLNTRVGTKRYMAPEVLDESLNKNHFQPYIMADIYSFGLIIWEMARRCITGGIVEEYQLPYYNMVPSDPSYEDMREVVCVKRLRPIVSNRWNSDECLRAVLKLMSECWAHNPASRLTALRIKKTLAKMVESQDVKI

SEQ ID NO: 8 - DNA, 2932 bp, Homo sapiens
gctccgcgccgagggctggaggatgcgttccctggggtccggacttatgaaaatatgcatcagtttaatactgtcttggaattcatgagatggaagcataggtcaaagctgtttggagaaaatcagaagtacagttttatctagccacatcttggaggagtcgtaagaaagcagtgggagttgaagtcattgtcaagtgcttgcgatcttttacaagaaaatctcactgaatgatagtcatttaaattggtgaagtagcaagaccaattattaaaggtgacagtacacaggaaacattacaattgaacaatgactcagctatacatttacatcagattattgggagcctatttgttcatcatttctcgtgttcaaggacagaatctggatagtatgcttcatggcactgggatgaaatcagactccgaccagaaaaagtcagaaaatggagtaaccttagcaccagaggataccttgccttttttaaagtgctattgctcagggcactgtccagatgatgctattaataacacatgcataactaatggacattgctttgccatcatagaagaagatgaccagggagaaaccacattagcttcagggtgtatgaaatatgaaggatctgattttcagtgcaaagattctccaaaagcccagctacgccggacaatagaatgttgtcggaccaatttatgtaaccagtatttgcaacccacactgccccctgttgtcataggtccgttttttgatggcagcattcgatggctggttttgctcatttctatggctgtctgcataattgctatgatcatcttctccagctgcttttgttacaaacattattgcaagagcatctcaagcagacgtcgttacaatcgtgatttggaacaggatgaagcatttattccagttggagaatcactaaaagaccttattgaccagtcacaaagttctggtagtgggtctggactacctttattggttcagcgaactattgccaaacagattcagatggtccggcaagttggtaaaggccgatatggagaagtatggatgggcaaatggcgtggcgaaaaagtggcggtgaaagtattctttaccactgaagaagccagctggtttcgagaaacagaaatctaccaaactgtgctaatgcgccatgaaaacatacttggtttcatagcggcagacattaaaggtacaggttcctggactcagctctatttgattactgattaccatgaaaatggatctctctatgacttcctgaaatgtgctacactggacaccagagccctgcttaaattggcttattcagctgcctgtggtctgtgccacctgcacacagaaatttatggcacccaaggaaagcccgcaattgctcatcgagacctaaagagcaaaaacatcctcatcaagaaaaatgggagttgctgcattgctgacctgggccttgctgttaaattcaacagtgacacaaatgaagttgatgtgcccttgaataccagggtgggcaccaaacgctacatggctcccgaagtgctggacgaaagcctgaacaaaaaccacttccagccctacatcatggctgacatctacagcttcggcctaatcatttgggagatggctcgtcgttgtatcacaggagggatcgtggaagaataccaattgccatattacaacatggtaccgagtgatccgtcatacgaagatatgcgtgaggttgtgtgtgtcaaacgtttgcggccaattgtgtctaatcggtggaacagtgatgaatgtctacgagcagttttgaagctaatgtcagaatgctgggcccacaatccagcctccagactcacagcattgagaattaagaagacgcttgccaagatggttgaatcccaagatgtaaaaatctgatggttaaaccatcggaggagaaactctagactgcaagaactgtttttacccatggcatgggtggaattagagtggaataaggatgttaacttggttctcagactctttcttcactacgtgttcacaggctgctaatattaaacctttcagtactcttattaggatacaagctgggaacttctaaacacttcattctttatatatggacagctttattttaaatgtggtttttgatgcctttttttaagtgggtttttatgaactgcatcaagacttcaatcctgattagtgtctccagtcaagctctgggtactgaattgcctgttcataaaacggtgctttctgtgaaagccttaagaagataaatgagcgcagcagagatggagaaatagactttgccttttacctgagacattcagttcgtttgtattctacctttgtaaaacagcctatagatgatgatgtgtttgggatactgcttattttatgatagtttgtcctgtgtccttagtgatgtgtgtgtgtctccatgcacatgcacgccgggattcctctgctgccatttgaattagaagaaaataatttatatgcatgcacaggaagatattggtggccggtggttttgtgctttaaaaatgcaatatctgaccaagattcgccaatctcatacaagccatttactttgcaagtgagatagcttccccaccagctttattttttaacatgaaagctgatgccaaggccaaaagaagtttaaagcatctgtaaatttggactgttttccttcaaccaccattttttttgtggttattatttttgtcacggaaagcatcctctccaaagttggagcttctattgccatgaaccatgcttacaaagaaagcacttcttattgaagtgaattcctgcatttgatagcaatgtaagtgcctataaccatgttctatattctttattctcagtaacttttaaaagggaagttatttatattttgtgtataatgtgctttatttgcaaatcaccc

SEQ ID NO: 9 - protein, 232 aa, Homo sapiens
MERCPSLGVTLYALVVVLGLRATPAGGQHYLHIRPAPSDNLPLVDLIEHPDPIFDPKEKDLNETLLRSLLGGHYDPGFMATSPPEDRPGGGGGAAGGAEDLAELDQLLRQRPSGAMPSEIKGLEFSEGLAQGKKQRLSKKLRRKLQMWLWSQTFCPVLYAWNDLGSRFWPRYVKVGSCFSKRSCSVPEGMVCKPSKSVHLTVLRWRCQRRGGQRCGWIPIQYPIISECKCSC

SEQ ID NO: 10 - DNA, 1557 bp, Homo sapiens
  misc_feature (430)..(430): n is a, c, g, or t
gagctccggcgggtcagccggactgtcggcttcccggggcatctgggtccggcggggcacagccctgggcgctgccgaagccgccgccgccgcctccgcggcgagtacaggcggcttcccccggagcctgtgcagctccagctcctcgggggtggagaagtggggggtgggggtgatgtatggggggaagaagggggaggggccaaccccgagagagtcagtggtttccatggtgatggagctgaaagtgcaggaaatttaaaggcttggaccctgcgagacagacaaaccggtgccaacgtgcgcggacgccgccgccgccgccgccgctggagtccgccgggcagagccggccgcggagcccggagcaggcggagggaagtgcccctagaaccagctcagccagcggcgcttgcacagagcggccggncgaagagcagcgagaggaggaggggagagcggctcgtccacgcgccctgcgccgccgccggcccgggaaggcagcgaggagccggcgcctcccgcgccccgcggtcgccctggagtaatttcggatgcccagccgcggccgccttccccagtagacccgggagaggagttgcggccaacttgtgtgcctttcttccgccccggtgggagccggcgctgcgcgaagggctctcccggcggctcatgctgccggccctgcgcctgcccagcctcgggtgagccgcctccggagagacgggggagcgcggcggcgccgcgggctcggcgtgctctcctccggggacgcgggacgaagcagcagccccgggcgcgcgccagaggcatggagcgctgccccagcctaggggtcaccctctacgccctggtggtggtcctggggctgcgggcgacaccggccggcggccagcactatctccacatccgcccggcacccagcgacaacctgcccctggtggacctcatcgaacacccagaccctatctttgaccccaaggaaaaggatctgaacgagacgctgctgcgctcgctgctcgggggccactacgacccaggcttcatggccacctcgccccccgaggaccggcccggcgggggcgggggtgcagctgggggcgcggaggacctggcggagctggaccagctgctgcggcagcggccgtcgggggccatgccgagcgagatcaaagggctagagttctccgagggcttggcccagggcaagaagcagcgcctaagcaagaagctgcggaggaagttacagatgtggctgtggtcgcagacattctgccccgtgctgtacgcgtggaacgacctgggcagccgcttttggccgcgctacgtgaaggtgggcagctgcttcagtaagcgctcgtgctccgtgcccgagggcatggtgtgcaagccgtccaagtccgtgcacctcacggtgctgcggtggcgctgtcagcggcgcgggggccagcgctgcggctggattcccatccagtaccccatcatttccgagtgcaagtgctcgtgctagaactcgggggccccctgcccgcacccggacacttgatcctcgagctc

SEQ ID NO: 11 - protein, 232 aa, Mus musculus
MERCPSLGVTLYALVVVLGLRAAPAGGQHYLHIRPAPSDNLPLVDLIEHPDPIFDPKEKDLNETLLRSLLGGHYDPGFMATSPPEDRPGGGGGPAGGAEDLAELDQLLRQRPSGAMPSEIKGLEFSEGLAQGKKQRLSKKLRRKLQMWLWSQTFCPVLYAWNDLGSRFWPRYVKVGSCFSKRSCSVPEGMVCKPSKSVHLTVLRWRCQRRGGQRCGWIPIQYPIISECKCSC

SEQ ID NO: 12 - DNA, 990 bp, Mus musculus
gcggccgcgccttcccaagtagagcggcgggggggaattgcgaccaactcgtgcgcgtcttctgcgccgcggcggcaggccgctgcgcgaacggctctcctcgcagctcatgctgcctgccctgcgcctgctcagcctcgggtgagccacctccggaggaccggggagcgcggcacgcgcggactcggcgtgctctcctccggggacgcgggacgaagaggcagccccggggcgcgcgcgggaggcatggagcgctgccccagcctgggggtcaccctctacgccctggtggtggtcctggggctgcgggcagcaccagccggcggccagcactatctacacatccgcccagcacccagcgacaacctgcccttggtggacctcatcgaacatccagaccctatctttgaccctaaggagaaggatctgaacgagacgctgctgcgctcgctgctcgggggccactacgacccgggctttatggccacttcgcccccagaggaccgacccggagggggcgggggaccggctggaggtgccgaggacctggcggagctggaccagctgctgcggcagcggccgtcgggggccatgccgagcgagatcaaagggctggagttctccgagggcttggcccaaggcaagaaacagcgcctgagcaagaagctgaggaggaagttacagatgtggctgtggtcacagaccttctgcccggtgctgtacgcgtggaatgacctaggcagccgcttttggccacgctacgtgaaggtgggcagctgcttcagcaagcgctcctgctctgtgcccgagggcatggtgtgtaagccatccaagtctgtgcacctcacggtgctgcggtggcgctgtcagcggcgcgggggtcagcgctgcggctggattcccatccagtaccccatcatttccgagtgtaagtgttcctgctagaactcgggggggccccctgcccgcgcccagacacttgatggatcc

(19) United States
(12) Patent Application Publication
(10) Pub. No.: US 2007/0036769 A9
(43) Pub. Date: Feb. 15, 2007

(54) BMP pathway methods and compositions

(76) Inventors: Linheng Li, Leawood, KS (US); Xi He, Leawood, KS (US)

Correspondence Address: POLSINELLI SHALTON WELTE SUELTHAUS P.C., 700 W. 47TH STREET, SUITE 1000, KANSAS CITY, MO, 64112-1802, US

(21) Appl. No.: 10/860,501

(22) Filed: Jun. 3, 2004

(51) Int. Cl. A61K 48/00; C12N 5/08; C12N 15/86

(52) U.S. Cl. 424/93.21; 435/456; 435/366; 514/44

(57) ABSTRACT

The present invention relates to mutant BMP intestinal stem cells (ISCs), with these mutant ISCs possessing an inactive Bmpr1a receptor in which BMP binding is substantially inhibited. The present invention relates to vectors which comprise mutant Bmpr1a nucleic acid sequences, whereby the vectors can be used to promote an increase in the number of ISCs in vivo or in vitro.

### FIELD OF INVENTION

The present invention relates to methods and compositions for studying intestinal stem cell (ISC) populations in vivo and in vitro, whereby mutant intestinal stem cells having mutant Bmpr1a nucleic acid receptors can be formed. Systems and tools are provided which show that BMP helps to control or influence self-renewal, proliferation, differentiation, and apoptosis in intestinal stem cells and mature intestinal cells, including progenitor cells and differentiated adult cells. The invention also relates to a mutant Bmpr1a mouse that can be used as an animal model for the study of human juvenile intestinal polyposis (JPS).

### BACKGROUND OF INVENTION

The gastrointestinal (GI) system has a well-organized developmental architecture which includes intestinal stem cells (ISCs), transient amplifying (TA) progenitors, functionally mature cells, and apoptotic cells all of which are confined to identifiable regions in each crypt/villus unit. This developmental architecture forms a sequential array of compartments (or zones) which promote self-renewal of stem cells, proliferation of progenitors, differentiation of progenitors to mature cells, and apoptosis in the mature cells, as illustrated in FIG. 1F. The developmental architecture or microenvironment is generally divided into three functional compartments, based upon stages of stem cell development, including (1) self-renewal, (2) expansion or transient amplification, and (3) differentiation zones. These zones correspond to the developmental state of the ISCs. As such, it is desired to know what controls and determines the different zones.

As a result of the sequential assay of the zones, the GI system provides an excellent model for the study of stem cell development and the related microenvironment. Greater understanding of the molecular mechanisms responsible for ISC proliferation, differentiation, and development can be used for the development of therapeutic tools for treatment of intestinal disorders. Specifically, the development of diagnostic and treatment modalities for tumors and polyps formed in the intestine are needed. While it is known that abnormally proliferating intestinal cells can lead to tumorigenesis, an understanding of the molecular mechanisms which control and influence proliferation can lead to methods and compositions for diagnosing and treating intestinal tumors.

The mucosa of the small intestine is involved in nutrient absorption and is characterized by evaginations into the villi, and by short tubular inaginations into crypts. The villi are projections into the lumen and are covered predominantly with mature, absorptive enterocytes, along with occasional mucous-secreting goblet cells. These cells survive only a few days, die through apoptosis, and are shed into the lumen to become part of the ingesta to be digested and absorbed by the body. The crypts of Lieberkuhn are moat-like inaginations of the epithelium around the villi. At the base of the crypts are the ISCs, which continually divide and provide the source for all epithelial cells in the crypts and villi.

The crypts, located at the base of the villus, provide a protective site for stem cells. Intestinal mucosa is lined by simple columnar epithelium, which consists primarily of enterocytes, absorptive cells, with scattered goblet cells, and occasional enteroendocrine cells. In the crypts, the epithelium also includes paneth cells and intestinal stem cells. Intestinal cells may be divided categorically into the following: ISCs, paneth cells, goblet cells, enterocytes (absorptive cells), enteroendocrine, and brunner's glands cells. ISCs are multipotent, undifferentiated cells that fundamentally retain the capacity for cell division and regeneration to replace various intestine cells that undergo apoptosis and die. It is desired to know what signals control differentiation of the ISC into the various differentiated adult cells.

One of the daughter cells from each stem cell division is retained as a stem cell, while the other becomes committed to differentiate along one of four lineage pathways into one of the following differentiated cells: enterocyte, enteroendocrine cell, goblet cell, or paneth cell. Cells in the enterocyte lineage continue to divide as they migrate away from the crypts and to the villi. Migration of intestinal stem cells results in differentiation into the mature absorptive cells, with the ISCs differentiating into enterocyte, enteroendocrine, goblet, and paneth cells. How the sequential events of ISC development are regulated and, particularly, what signal pathways are involved in controlling the self-renewal of ISCs, are largely unknown.

ISCs are thought to be located in the fourth or fifth position from the bottom of each crypt in the small intestine. ISCs are also found at the bottom of the table region of the villi of the large intestine. Unlike adult stem cells in other tissue systems, and for an unknown reason, the currently identified ISCs have a relatively high rate of cell proliferation. This provides a general system for studying stem cells and the regulatory mechanisms that govern their proliferation, growth, and differentiation.

Substantial evidence indicates that the bone morphogenic protein (BMP) pathway may be involved in regulation of morphogenesis and postnatal regeneration of GI development; however, the molecular mechanism(s) of BMP involvement in the GI tract remains for elucidation. BMPs belong to the TGF-β super family and are found in species ranging from flies to mammals. The BMP signal is known to be important in cell fate determination and pattern formation during embryogenesis and in the maintenance of tissue homeostasis in the adult. According to the current model, BMP2 and 4 function by first binding to a type-II receptor and then by recruiting type I receptor A or B (Bmpr1a or b, also referred to as ALK3 (activin A receptor, type II-like kinase 3 or 6), respectively).

The regulatory signals for modulation of ISC growth, proliferation, and differentiation have been largely uncharacterized. At present, it is known that Bmpr1a receptors on stem cells and differentiated cells derived therefrom, including ISCs, bind BMPs. While BMP- and Noggin-mediated regulation of embryonic development has been determined, the interactions between the Bmpr1a receptor on stem cells and regulators such as BMP and Noggin in adult tissues in general, and intestinal tissue in particular, have not been completely characterized. Specifically, Bmpr1a, BMP, and Noggin activities in the intestinal niche, and the resultant effects upon intestinal cell growth, proliferation, self-renewal, differentiation, and apoptosis have remained unknown.

It is desired to have a viable conditional mutant Bmpr1a organism that possesses cells having inactive Bmpr1a cell surface receptors encoded by a mutant Bmpr1a gene for investigation of the impact of Bmpr1a upon ISC growth, self-renewal, proliferation, differentiation, and apoptosis in vivo. The inactive Bmpr1a receptor is unresponsive to BMP or Noggin signaling. Moreover, model Bmpr1a mutant organisms for in vivo and in vitro analyses of ISCs are desired. In particular, an animal model for study of human Juvenile Polyposis Syndrome (JPS) is desired. It is desired to develop compositions and methods for the induction of ISC self-renewal, proliferation, growth, and differentiation within the intestinal tissue architectural structure. Methods for controlling the intestinal pathway are desired. Also, identification of cell markers, including cell surface markers, are desired. It is especially desired to identify distinct markers, which can be used to identify various types of cells in the tissue. These markers could be used to isolate ISC. Related to this, a useful molecular biology tool would be a viable Bmpr1a conditional knock-out mouse, since null homozygous Bmpr1a allele-containing mutant mice are embryonically lethal, dying at embryonic day 8 without mesoderm formation. At present, lethality of the null Bmpr1a mutant mouse has hampered investigation of Bmpr1a cell receptors and their role in modulating ISC expansion and differentiation in postnatal stages of development.

Molecular biology tools are desired for studying Bmpr1a. Desired tools include mutant Bmpr1a nucleic acid sequences, inactive Bmpr1a polypeptides, Bmpr1a antisense nucleic acid sequences, isolated Noggin polypeptides, vectors containing mutant Bmpr1a nucleic acid sequences, anti-Bmpr1a receptor antibodies, anti-BMP antibodies, PTEN family nucleotide sequences, proteins, antibodies, and fragments thereof. Kits utilizing Bmpr1a, BMP, and Noggin polypeptide and nucleic acid markers, and mutants thereof, for detection and quantitation of these markers in intestinal tissue are also desired. In vitro intestinal tissue and cell cultivation systems are desired for expansion of wild type (Wt) ISCs and mutant ISCs containing inactive Bmpr1a receptor polypeptides. Methods for making and using the foregoing Bmpr1a genes, Bmpr1a polypeptides, vectors, Bmpr1a mutant organisms, ISCs, tumors, and molecular biology tools are desired.

### SUMMARY OF INVENTION

The present invention relates to compositions and methods which can be used to influence proliferation, self-renewal, cell differentiation, and apoptosis in intestinal cells and tissue, both in vivo and in vitro. The compositions and methods are directed to altering the Bmpr1a and BMP interaction, as well as related proteins and polypeptides influenced by the Bmpr1a and BMP interaction. As such, the compositions and methods are used to inhibit BMP and Bmpr1a interaction, and PTEN pathway proteins. The methods and compositions can be utilized in isolated cells, isolated tissue cultures, or in vivo in organisms, such as in a mouse. Phenotypic results observed include tumor and polyp formation, altered cell differentiation so that there is an increase in mucosal progenitor cells, and inhibited apoptosis in differentiated intestinal cells. This information can be used to create models, kits, and cultures useful in studying and treating intestinal polyposis in humans, including juvenile polyposis. The compositions and methods can also be used in conjunction with procedures for screening drugs.

A pathway is disclosed which influences self-renewal, differentiation, and apoptosis in ISC and intestinal cells. The pathway is illustrated in FIG. 18. The pathway can be used as part of a method to control cells in vivo or in vitro. Further, the pathway provides the basis for developing in vitro cell development systems. A population of ISCs with increased self-renewal are identified by various markers, including P-PTEN⁺, P-AKT⁺, nuclear accumulated β-catenin, 14-3-3 ζ, and Tert⁺. A population of transient amplifying progenitors, which are proliferating, are identified by markers Ki67⁺ and Brd-U⁺. Markers for determining whether intestinal cells are mutagenized are identified. The markers include Ki67, P-PTEN, PTEN, AKT, P-AKT, Tert, β-catenin, P-Smad1,5,8, BMP, Noggin, Bmpr1a, BAD, P-BAD, 14-3-3ζ, and combinations thereof. The markers for identifying inhibited apoptosis in intestinal cells are BAD and Tunel.

In vitro intestinal tissue samples having mutant cell populations are identified. The tissue samples are formed by mutagenizing the sample in vitro or identifying an in vivo sample and removing the in vivo sample for in vitro uses. The tissue samples are useful for studying ISC and intestinal cell populations. In the samples, BMP in individual cells is blocked from binding Bmpr1a. This results in an increased number of ISCs self-renewing, and an increased amount of P-PTEN. Also, there is an increased amount of P-PTEN and P-AKT mucosal progenitor cells. The isolated stem cell population is characterized as being Bmrpr1a⁺, Noggin⁺, and P-PTEN⁺. All of these cells can be fixed in vitro. Noggin can be used as a marker to isolate ISC, which has potential in tissue regeneration.

A Bmpr1a gene, or nucleotide sequence, is isolated, or obtained from a third party. The Bmpr1a gene or nucleotide sequence can be mutagenized or used to form a conditional mutant. Regardless, the Bmpr1a gene is amplified and used to form vectors for use in transfecting cells. Additionally, other genes or nucleotide sequences can be used. BMP, Noggin, PTEN, p27, 14-3-3ζ, BAD, or any other PTEN pathway genes, for example, can be utilized to alter cell proliferation, differentiation, and apoptosis in intestinal cells.

The selected nucleotide sequence can be a Wt or a fragment of the Wt gene. In the alternative, the Wt or fragment can be mutated. Further, Wt homologous nucleotide sequences or degenerate variants may be used. In place of a DNA nucleotide sequence, RNA nucleotide sequences, which are transcribed or related to the selected nucleotide sequence, can be used.

Vectors can be formed from one or more of the above nucleotide sequences. The vectors can be used to make a conditional mutant or can be used to nonconditionally mutagenize cells. To make a conditional mutant the vector will include a selected nucleotide sequence and at least one recombination site. Again, the nucleotide sequence can include Wt, mutant, homologous, degenerate variants, fragments, isolated exons, and any of a variety of nucleotide sequences related to the selected gene or nucleotide sequence. The nucleotide sequence can be inserted into a variety of vectors including a gene expression cassette, a plasmid, an episome, or a viral nucleic acid sequence. Preferably, in the conditional mutant the nucleotide sequence will express a functional protein until such time as it is desired to knock-out expression or cause expression of a nonfunctional protein. A preferred vector includes a Bmpr1a nucleic acid sequence and recombination sites, which produce knock-out organisms. Examples of suitable recombination sites include LoxP and FRT. The vectors can be prokaryotic or eukaryotic dependent upon the organism to be transfected. Recombination will occur in a transfected cell, causing a selected gene to be knocked out when activated. If the selected gene is the Bmpr1a nucleotide sequence this will promote an increase in the ISC population in vitro or in vivo.

Recombination will be facilitated by the vector. Upon activation the recombinant will cut or knock-out the nucleotide sequence. If a mutant nucleotide sequence is used, recombination will result in replacement of the Wt gene or sequence with the mutant. Typically, this occurs in the nucleus of the cell. An alternative is to use a plasmid to “flood” the cytoplasm and produce increased amounts of a selected polypeptide.

The vector, preferably is an inducible Cre expression vector, with Lox recombination sites flanking the target gene. The vector can include multiple recombination sites, and markers, such as LacZ, along with a selected target gene. As such, the method of forming the conditional mutant is initiated by forming a vector which includes the Bmpr1a, BMP, Noggin, or PTEN pathway nucleotide sequence through transfection of embryonic stem cells. This vector-mediated method for obtaining a Bmpr1a mutant organism will include use of the inducible Cre/Lox system, whereby the Bmpr1a gene is flanked by LoxP sites. In particular, mice can be transfected with this Bmpr1a vector. Specifically, pre-excision and post-excision Mx1-Cre⁺, Bmpr1a^{fx/fx} mice are formed using the vector. A Bmpr1a post-excision knock-out mouse results, wherein a portion of the Bmpr1a gene, such as Exon 2, has been substantially eliminated through Cre recombinase-mediated excision of Exon 2, resulting in expression of inactive Bmpr1a receptor polypeptide, where binding to BMP is substantially inhibited.

If differentiated adult tissue is to be mutagenized, the mutant will likely not need to be conditional. Instead, the vector will include a nonfunctional Bmpr1a mutant sequence that encodes an inactive Bmpr1a receptor polypeptide. Alternatively, the vector can include a promoter, and a stem cell activator, such as a nucleotide sequence encoding antisense Bmpr1a, P-PTEN, activated AKT, Noggin, or activated PI3K. Alternatively, the vector can contain a promoter, and a gene such as PTEN, AKT, GSK-3, cyclin D1, Tert, PI3K, Smad1, 5, 8, p27, or derived mutant genes. The tissue can be derived from any mammal.

The vector containing a conditional recombination site-flanked gene is used to transfect a selected cell, preferably an embryonic stem (ES) cell. The ES cell can be placed in an adoptive mother so that the transfected stem cell develops into a conditional mutant embryo and then a conditional mutant adult. Alternatively, the vector can be used to transfect an isolated cell or tissue culture for development in vitro. This allows intestinal cells, for example, to be studied in a tissue culture. As such, mutant intestinal cells can be formed by transfection with the vector, or as a result of clonal formation during gestation resulting from a transfected embryonic stem cell.

The present invention also relates to a mutant ISC containing an isolated mutant Bmpr1a nucleic acid sequence which encodes an inactive Bmpr1a receptor. The isolated mutant Bmpr1a nucleic acid sequence can contain a mutation such as a frame shift, substitution, loss of function, knock-out deletion, or conventional deletion mutations. The present invention also relates to a mutant ISC containing a truncated Bmpr1a nucleic acid sequence, which is lacking Exon 2 of the Bmpr1a receptor nucleic acid sequence, wherein the truncated sequence encodes an inactive Bmpr1a polypeptide. The mutant ISC can contain an inactivated Bmpr1a receptor polypeptide, wherein Bmpr1a binding to BMP is substantially inhibited. A mutant ISC containing an antisense oligonucleotide that operably hybridizes with a Bmpr1a mRNA sequence to inhibit intracellular translation of a Bmpr1a polypeptide is also contemplated. Alternatives to using a vector to knock-out the Bmpr1a receptor are available. Such alternatives include compositions, which specifically attack the Bmpr1a receptor to render it nonfunctional. Available compositions include RNAi molecules and various chemical agents. Transfected intestinal cells are contemplated. The intestinal cells include mutants, as well as pre-recombination sequences.

Intestinal cells containing the aforementioned pre or post Bmpr1a mutation can be selected from the following: intestinal epithelial, intestinal epithelial stem, mesenchymal, paneth, goblet, polyp, hemartoma, tumor, villus, crypt, and basement membrane cells. The intestinal cell containing the Bmpr1a mutation can be resting, self-renewing, proliferating, transient amplifying, differentiating, or apoptotic cells. The intestinal cells can be specifically isolated from the following organs, a stomach, intestine, digestive tract, duodenum, or colon cell. A mutant Bmpr1a gene or sequence can be inserted into the intestinal stem cell by transfection with a vector, electroporesis, biolistic particle delivery, liposome encapsulation, micro-vessel encapsulation, particle bombardment, or a microinjection method.

The transfected conditional mutant embryonic stem cells can be used to form adult conditional mutants. Transfected mice are formed whereby the mutant can be activated by injection of PolyI:C. Activation will result in the mouse having mutagenized intestinal tissue cells. There are two resultant organisms, the conditional mutant and the activated mutant. Tissue samples can also be conditional or activated mutants, with the tissue samples derived from a variety of organisms, including mammals, especially humans and mice.

Antibodies to the Bmpr1a polypeptide can be formed, along with fragments thereof. An anti-Bmpr1a mutant antibody is specifically part of the invention, wherein the antibody binds an epitope recognized in the truncated polypeptide sequence of SEQ ID NO 5. Also contemplated is an ISC comprising an isolated antibody, such as anti-Bmpr1a antibody, anti-BMP antibody, and fragments thereof, whereby the antibody induces intestinal stem cell proliferation in vitro or in vivo by inhibiting BMP binding to Bmpr1a receptor. Alternatively, antibodies such as anti-Bmpr1a antibodies, anti-BMP antibodies, and fragments thereof, can be utilized in the in vitro intestinal stem cell cultivation system to cause intestinal stem cell proliferation. Additionally, mutant Bmpr1a stem cells may be cultivated in in vitro culture medium since the mutant stem cells comprise inactive Bmpr1a cell receptors which are unresponsive to inhibitory BMP signals.

Hybridomas for producing the antibodies can be formed. The hybridomas will express an antibody to the selected protein, such as the Bmpr1a receptor.

Kits and methods for the detection, quantitation, and monitoring of Wt and mutant polypeptides and nucleic acid sequences of Bmpr1a, BMP, Noggin, PTEN, P-PTEN, AKT, PAKT, Tert, β-catenin, Ki67, p27, Smad1,5,8, tubulin, Chromogin A, BAD, PBAD, and FAK markers in in vitro and in vivo intestinal cells and tissues are developed. For identification of polypeptides, antibodies to the foregoing markers are used; and for identification of the foregoing nucleic acid sequences, nucleic acid probes are used. In particular, detection of the presence of these polypeptide and nucleic acid markers in intestinal stem cells is contemplated.

In vitro intestinal stem cell cultivation systems are made, wherein an intestinal stem cell population proliferates. The system possesses an intestinal tissue section or an isolated intestinal stem cell population with at least 10⁴ cells in culture medium, and an isolated Noggin polypeptide that operably binds to Bmpr1a cell receptors, wherein Bmpr1a receptor binding to BMP is substantially inhibited.

Finally, methods for increasing intestinal stem cell population numbers in vitro and in vivo are also within the scope of the invention. Methods include the following: formation of post-excision Mx1-Cre⁺Bmpr1a^{fx/fx} knock-out mutant organisms; formation of post-excision Mx1-Cre⁺Bmpr1a^{fx/fx} Z/EG knock-out mutant organisms; in vitro cultured Bmpr1a mutant intestinal stem cells; in vitro cultured intestinal Wt and Bmpr1a mutant tissue; and in vitro cultivated Wt intestinal stem cells, with either Bmpr1a antisense oligonucleotide, antibody (anti-Bmpr1a, anti-BMP), or Noggin activators.

Because of the similarity of histopathology between the Bmpr1a mutant mouse and human JPS, this mouse may serve as a workable animal model for investigation of the molecular control mechanisms responsible for the JPS disorder. In support, mutations in the Bmpr1a gene have been found in human patients having a subset of JPS with features of hemartomas and polyps throughout the digestive tract, including stomach, duodenum, and colon.

Mechanistically, the Bmpr1a mutant mouse system can be used as a model for study of the pivotal biochemical pathways and regulator molecules responsible for causing the JPS disorder. Based upon results obtained in the Bmpr1a mutant mouse, the BMP signal, which formed a Noggin/BMP-receptor dependent activity gradient, was discovered to play an essential role in maintaining the stability of the ISC compartment. Mutations in the Smad4 gene, which encode a down stream transcriptional factor for the BMP/TGF-β pathways, also have been reported to result in JPS in humans, but this factor only accounts for a subset of JPS cases. PTEN, an inhibitor of the PI3K/AKT pathway, is additionally responsible for some JPS cases. Since PI3K/AKT activity has been proposed to be subject to regulation by the BMP signal pathway, it was postulated herein that a common link in these different types of JPSs might be the PI3K pathway. The Bmpr1a mutant mouse can thus serve as a model for the study of the BMP/TGF-β, PI3K, and other pathways and their roles in causation of JPS-derived disorders.

### BRIEF DESCRIPTION OF DRAWINGS

The application file contains at least one drawing executed in color. Copies of this patent application publication with color drawing(s) will be provided by the Office upon request and payment of the necessary fee.

FIG. 1A shows anti-BrdU staining in intestinal tissue 22 days after ISC is labeled, whereby the location of ISCs relative to paneth cells in the crypt region is identified;

FIG. 1B shows the crypt bottom, which is illuminated by granules containing lysozyme recognized by an anti-lysozyme antibody so that the ISCs relative to the crypt cells are identified;

FIG. 1C shows that the stem cell appears in red at the bottom of the villus in the schematic diagram;

FIG. 1D shows BMP4-LacZ expression in the villus, as indicated by blue LacZ staining and an eosin counterstain, BMP4 expression was detected throughout the mesenchymal cells, and particularly in cells adjacent to positions where ISCs were located, such as at the black arrow;

FIG. 1E shows that BMP4 was detected in mesenchymal cells (MC) in the crypt, in cells adjacent to ISCs recognized by Brd-U;

FIG. 1F shows the stem cell position diagrammatically to the MC, the stem cell is colored red, and the MC colored green in the diagram;

FIG. 1G shows that Noggin expression (shown by blue) was restricted to the basement membrane region adjacent to the crypt; Noggin appeared in ISCs, where the white arrows indicate stem cell location;

FIG. 1H shows LacZ expression was observed in the stem cell located at the right arrow point in the villus, with no expression observed in the upper crypt;

FIG. 1I is a diagram that shows the position of the stem cell in blue, with Noggin appearing in blue at the base of the villus;

FIG. 1J shows that the expression of Bmpr1a receptor protein is found in epithelial cells with the protein levels varying in different crypt/villus regions;

FIG. 1K shows co-staining for Bmpr1a and 14-3-3ζ, whereby Bmpr1a was highly expressed in ISCs as shown by co-staining with an ISC marker 14-3-3ζ;

FIG. 1L shows that the Bmpr1a receptor activity is illustrated diagrammatically in red in the villus illustration;

FIG. 1M shows a stain for P-Smad1,5,8, whereby P-Smad1,5,8 is throughout the villus and in ISC;

FIG. 1N shows a co-stain for P-Smad1,5,8 and Brd-U in the crypt, where P-Smad is shown in relation to the ISC;

FIG. 1O is a diagrammatic illustration of P-Smad1,5,8 distribution;

FIG. 2 shows a graphical depiction of relative expression levels of BMP4, Bmpr1a, and Noggin, and compartmentalized BMP activity across the villus, with a diagram of the array of zones for stem cells undergoing proliferation and self-renewal, differentiation, and apoptosis, where the stem cells are situated adjacent to the paneth cells, later becoming epithelial cells in the differentiation zone, and ultimately becoming apoptotic at the tip of the lumen;

FIG. 3 depicts whole intact and cross-sectional stained views of stomach and intestine (large and small) with GFP expression patterns for tissue cross-sections obtained after PolyI:C induced LacZ inactivation;

FIG. 3A shows PolyI:C induced LacZ inactivation in intestine, where GFP expression patterns appear clonally in the crypt/villus unit, with FIG. 3B diagramatically depicting GFP staining, with each clonal villus is indicated in green as opposed to non-marked blue regions;

FIGS. 3C and 3D show Bmpr1a mutant whole mounts of small intestine in FIG. 3C and sections indicating polyp formation and tumors in FIG. 3D;

FIGS. 3E and 3F show Ki67 staining with primary anti-Ki67 antibody and AEC-conjugated secondary antibody of Wt and polyp sections respectively, where ISCs in the Wt are labeled with black arrows, the polyp shows increased Ki67;

FIGS. 3G and 3H shows small intestine whole mounts, with cross-sectional stain view in FIG. 3F;

FIGS. 4A and 4B show P-Smad1,5,8 staining of Wt versus tumor region staining respectively;

FIGS. 4C and 4D show P-PTEN staining of Wt versus polyp regions;

FIGS. 4E and 4F shows P-AKT staining of Wt and polyp regions respectively;

FIGS. 5A and 5B show β-catenin staining of Wt versus polyp regions respectively;

FIGS. 5C and 5D show Tert staining of Wt versus polyp regions respectively with ISCs depicted at the black and red arrows;

FIG. 6A shows Actin, P-AKT, and P-PTEN expression cells for Wt and Bmpr1a mutant mice;

FIG. 6B shows electrophoretic gel marker expression for control mice versus Noggin, BMP4, and Noggin+Ly294002 mice for the following markers: PTEN, P-PTEN, AKT, P-AKT, Tert, β-catenin, and Actin;

FIGS. 7A, 7B, and 7C show P-PTEN staining of an ISC in FIG. 7A; BrdU-R staining of the ISC in FIG. 7B; and merged staining in FIG. 7C;

FIGS. 7D, 7E, and 7F show primary and secondary cells with AKT-S473 and BrdU-R staining in FIG. 7D and FIG. 7E, respectively, and merged staining pattern in FIG. 7F;

FIG. 7G shows β-catenin and N-Cad staining of ISCs and paneth cells; β-catenin staining of an ISC is shown in FIG. 7H; P-PTEN staining of the same ISC region is shown in FIG. 71;

FIGS. 7J, 7K, and 7L show P-PTEN, Tert (Telomerase reverse transcriptase) staining of ISC, and merged staining in FIGS. 7J, 7K, and 7L respectively, white arrows show the paneth cell as a marker geographical point of reference, β-catenin staining of an ISC alone is shown in FIG. 7K, and P-PTEN merged staining of the same stem cell region is shown in FIG. 7L;

FIGS. 7M, 7N, and 7O show P-PTEN, α-Tubulin, and merged staining of ISCs in interphase staining, respectively;

FIGS. 8A, 8B, and 8C show Wt staining patterns of P-PTEN, α-Tubulin, and merged patterns in anaphase, with arrows indicating a horizontal plane of cell division, respectively;

FIG. 8D shows α-Tubulin, γ, and P-PTEN staining depicting AEC primary and secondary cells with the horizontal plane of cell division of the secondary cell indicated by red arrows;

FIG. 8E shows a diagram of the secondary cell division illustrating the horizontal orientation of the spindle (green) in cell division;

FIGS. 8F and 8G show P-PTEN and α-Tubulin staining of tumor regions, with arrows indicating the direction of cell division;

FIGS. 8H and 8I show P-PTEN and α-Tubulin staining of tumor regions, with arrows indicating planes of cell division;

FIG. 8J shows α-Tubulin and γ-Tubulin staining of the metaphase cell;

FIG. 8K shows P-PTEN of the dividing cell;

FIG. 8L shows FAK staining of the stem cell;

FIG. 8M shows P-PTEN staining of the same stem cell depicted in FIG. 8L;

FIGS. 9A and 9B show Alcian blue staining to detect goblet cells in Wt and mutant intestine, respectively;

FIGS. 9C and 9D show PAS stain that was used to detect paneth cells in Wt and mutant intestine, respectively;

FIGS. 9E and 9F show alkaline phosphatase staining that is a marker for enterocytes in Wt and mutant intestine, respectively;

FIGS. 9G and 9H show anti-Chromgrin-A staining that was used to detect endocrine cells, indicated by a red arrow in Wt and mutant intestine, respectively;

FIGS. 91 and 9J show Wt and mutant tissue samples stained with Tunel, to show apoptotic activity in the lumen;

FIGS. 10A and 10B show BAD staining used to detect apoptotic cells in Wt and mutant intestine, respectively;

FIGS. 10C and 10D show Id2 is expressed predominantly in villi of Wt, but is significantly reduced in mutant mice;

FIGS. 10E and 10F Wt and mutant tissue was stained with P-LRP6, where P-LRP6 is predominantly expressed in crypts of Wt and mutant intestines;

FIGS. 10G and 10H show P-BAD staining that was used to detect non-apoptotic cells, indicated at the black arrows in Wt and mutant intestine, respectively;

FIGS. 10I and 10J show BMP signaling consequences and their disruption in Bmpr1a mutant intestinal sections, as anti-P-BAD was used to detect apoptotic and non-apoptotic cells, respectively;

FIG. 11A shows a schematic diagram illustrating the role of the localized BMP activity modulated by Noggin in the regulation of stem cell self-renewal, proliferation, lineage fate determination and differentiation, and apoptosis corresponding to physical regions along the villus;

FIG. 11B shows a pathway illustration of Noggin blockage of BMP activity through the following: phosphorylated P-PTEN, activating PI3K-AKT, leading to relocation of β-catenin, activation of Tert, and BAD conversion to P-BAD, which subsequently triggers proliferation;

FIG. 11C is an illustration of asymmetrical division versus symmetrical division and an indicator of crypt fission in the intestine;

FIG. 11D shows increased proliferation and crypt fission due to symmetrical cell division of ISCs, abnormal differentiation, and reduced apoptosis in tumor regions;

FIG. 12 shows co-staining of Bmpr1a and P-Smad1,5,8 markers with proliferation markers Ki67 and p27^{kip};

FIG. 12A shows Bmpr1a and Ki67 staining of micro villi, focusing on the proliferation zone which contains cells that are Ki67⁺ and stem cells which are Ki67;

FIG. 12B shows Bmpr1a and Ki67 staining of paneth cells, stem cells (Ki67⁻), and proliferation zone cells;

FIG. 12C shows P-Smad1,5,8 and Ki67 staining of villi;

FIG. 12D shows P-Smad1,5,8 and Ki67 staining of cells, with the crypt region depicted;

FIG. 12E shows p27^{kip} staining of villi;

FIG. 12F shows the stem cell juxtaposed adjacent to the paneth cell, near the proliferation zone;

FIGS. 13A and 13B show proliferating cells labeled by Ki67 in the red for Wt and Bmpr1a mutant intestinal tissue, respectively;

FIGS. 13C and 13D show Ki67 and P-PTEN staining for Wt and Bmpr1a mutant cells in the colon, respectively, where white arrows indicate PTEN staining;

FIG. 13E shows an intestine segment cell culture in vitro where beads containing Noggin or BMP were inserted by microinjection into the intestine segment;

FIG. 14 shows functional analysis of regulation of β-catenin and Tert mediated by AKT by BMP and Noggin using organ culture systems where control, BMP4, Noggin, and Noggin+L294002 conditions are depicted in photographs in vertical columns from left to right;

FIG. 14A shows that P-PTEN expression was activated by Noggin treatment and is not sensitive to Ly294002 treatment;

FIG. 14B shows that activated P-AKT became activated by Noggin treatment, but that this activation was inhibited by Ly294002;

FIG. 14C shows that β-catenin was activated and nuclearly localized by Noggin treatment and that this activation was inhibited by Ly294002;

FIG. 14D shows that Tert was activated by Noggin treatment and that this activation was inhibited by Ly294002;

FIG. 15A shows detection of P-PTEN in the villus and crypt;

FIG. 15B shows co-staining of cells retaining BrdU with P-PTEN in the small intestines, whereby P-PTEN is associated with ISC;

FIG. 15C shows co-staining of cells with Ki67 and P-PTEN in the colon, where ISC is not stained with Ki67;

FIG. 15D shows detection of P-PTEN in polyps;

FIG. 15E shows detection of P-AKT in the ISC of the villus and crypt of the small intestine;

FIG. 15F shows co-staining of Brd-U with P-AKT in small intestine;

FIG. 15G shows co-staining of P-AKT and Ki67 in ISC in colon tissue;

FIG. 15H shows detection of P-AKT in the crypts of polyps in mutant mice;

FIG. 15I shows co-staining of β-catenin and Brd-U in ISC in small intestine tissue;

FIG. 15J shows c-staining of β-catenin and P-PTEN in ISC in small intestine tissue;

FIG. 15K shows detection of nuclear-accumulated B-catenin in dividing ISCs, recognized by BrdU-R;

FIG. 15L shows detection of β-catenin in crypts of polyps in mutants;

FIG. 16A shows a small intestine section labeled with 14-3-3ζ, whereby Paneth and ISCs were labeled;

FIG. 16B shows co-staining P-PTEN with 14-3-3ζ in ISCs of the small intestine, whereby Paneth cells are distinguished from ISC;

FIG. 16C shows polyps of a small intestine section labeled with 14-3-34;

FIG. 16D shows ISC in small intestine crypt labeled with tert;

FIG. 16E shows ISC in small intestine crypt co-labeled with tert and P-PTEN;

FIG. 16F shows detection of tert in a polyp of mutant;

FIG. 17A shows a schematic diagram illustrating the role of the localized BMP activity modulated by Noggin in the regulation of stem cell self-renewal, proliferation, lineage fate determination and differentiation, and apoptosis corresponding to physical regions along the villus;

FIG. 17B shows an illustration of the regulatory roles of the BMP signal in each zone, and a cross talk between BMP signaling and Wnt signaling mediated by the PTEN-PI3K pathway; and,

FIG. 18 shows a schematic illustrating the regulatory roles of the compartmentalized BMP activity in each zone of self-renewal, proliferation, lineage fate determination, and apoptosis; and the role of Wnt signaling in promoting crypt fate but inhibiting the villus fate, a cross talk between BMP signaling and Wnt signaling mediated by the PTEN-PI3K-AKT pathway, and a balanced regulation between BMP and Wnt signaling over stem cells through a common factor, β-catenin.

### DETAILED DESCRIPTION

The present invention relates to a pathway for controlling self-renewal, proliferation, differentiation, and apoptosis in intestinal cells. Specifically, markers are identified which can be used for isolation of ISCs to distinguish between mutant and Wt cells, as well as a part of a screen for polyposis. Methods are developed which can be used to control cell development, including self-renewal, differentiation, proliferation, and apoptosis. The pathway for controlling ISC and intestinal cells and the biochemical constituents, in particular, proteins, have been identified.

The present invention relates to an organism, where Bmpr1a can be or has been made nonfunctional in intestinal tissue, and methods for making the organism, wherein intestinal cells of the organism can or do contain nonfunctional Bmpr1a nucleotide sequences that encode inactive Bmpr1a receptor polypeptides. A Bmpr1a knock-out organism or animal can be made through insertion of a mutant Bmpr1a nucleotide sequence into stem cells of the Wt animal by using a vector. The vector can contain a mutant or conditional mutant Bmpr1a sequence. The mutant can be conditionally activated, so it is preferred that the resultant organism is a conditional mutant used to study ISCs. Alternatively, a vector can be used to mutagenize ISCs in a mature organism. The proliferation, differentiation, and expression of the ISC population can be regulated in vivo and in vitro. This is beneficial because studies related to ISC self-renewal, proliferation, differentiation, and apoptosis can be conducted. The present invention also relates to blocking BMP regulation of various biochemical signals found in the crypt, villus, and lumen of the intestinal tissue. When BMP activity is blocked, the biochemical pathways are altered, causing increased proliferation of ISCs, altered differentiation, and reduced apoptosis. BMP can be blocked by knocking out the Bmpr1a receptor site, adding increased amounts of Noggin, mutagenizing Bmpr1a or BMP, or using an antibody to attack BMP or Bmpr1a.

Conditional Bmpr1a mutant ISCs are formed by transfecting embryonic stem cells, with the Bmpr1a gene, which is later rendered nonfunctional upon activation in a mature organism. The conditional mutation in a pre-recombination organism is maintained or is present throughout gestation. The Bmpr1a mutant cells can be formed in vivo. Alternatively, the ISCs can be isolated and treated in vitro to obtain Bmpr1a mutant ISCs. The conditional mutant ISCs can be studied and used as tools to better understand ISCs and the pathways influencing ISC differentiation, proliferation, and apoptosis. The conditional knock-out cells and organisms include pre-recombination and post-recombination cells and organisms. As the organism matures, the transfected embryonic stem cells will develop into transfected ISCs. In the adult organism, the ISC self renew, proliferate, and differentiate so that additional ISCs are formed, as well as TA progenitor cells, mucosal progenitor cells, columnar progenitor cells, followed by endocrine cells, paneth cells, goblet cells, and enterocytes. Because the mutation is clonal, all of these cells which can be transfected are conditional knock-outs. A post-recombination Bmpr1a mutant organism contains cells with inactive Bmpr1a receptors.

Formation of the knock-out or mutant organism is initiated by isolating a Wt Bmpr1a gene or nucleotide sequence. The isolated sequence can be any of a variety of structures, including genes, gene fragments, polynucleotides, oligonucleotides, and any nucleotide structure that can be substituted into the genome of a host and result in expression of a functional Bmpr1a polypeptide, until it is desired to mutagenize such structure. While it is preferred to isolate a gene, other hereditary units may be used. Homologous sequences are available, as are orthologs. Functional mutant sequences of Bmpr1a may be used. Gene fragments are available, as long as the organism properly develops prior to activation of the mutant. As such, any of a variety of nucleotide sequences can be used. The Bmpr1a gene is later defined herein.

The knock-out or mutant organism includes organisms formed from transfected embryonic stem cells and mature organisms transfected with a mutant Bmpr1a nucleotide sequence. If the embryonic stem cell is transfected, it will preferably be a conditional mutant. If an adult organism is transfected, a conditional mutant can be used, or the sequence can be directly mutagenized and not made conditional. The gene selected will preferably be isolated from the species in which the gene is to be used. For example, if the procedure is to be conducted in a mouse, then the Bmpr1a gene is preferably isolated from a mouse. Any of a variety of species, however, may be used. SEQ ID NO 1 is a suitable gene for use herewith.

As mentioned, the Bmpr1a gene or nucleotide sequence can be derived from a variety of species. Preferably, eukaryotic organisms are used. It is more preferred to use a mammalian gene, in particular mus musculus (mouse). The Wt Bmpr1a gene encodes a functional Bmpr1a receptor that can operatively bind to BMP.

BMP, Noggin, PTEN, p27, BAD, or any other PTEN pathway genes, for example, can be utilized to alter cell proliferation, differentiation, or apoptosis in intestinal cells. Any of the later compositions or structures that are mentioned as formed from or containing Bmpr1a, could be formed from any of the mentioned nucleotide sequences or related compositions.

The selected isolated nucleotide sequence is preferably amplified. This is done to provide a sufficient amount of Bmpr1a or other nucleotide sequence, so that vectors can be formed. It may be necessary to amplify one of the foregoing Bmpr1a nucleic acid sequences, which can be accomplished using standard PCR technology, prior to insertion into a vector. The Bmpr1a nucleotide sequence can be mutagenized or attached to at least two recombination sites. A mutation is made in the Wt Bmpr1a gene or nucleotide sequence, such that the sequence encodes an inactive Bmpr1a receptor polypeptide that is unable to bind with BMP. The resultant mutation can be a frame shift, point, substitution, loss of function, knock-out deletion or conventional deletion mutation. Importantly, the mutant sequence should remain substantially homologous to the Wt, but render the resultant gene nonfunctional. A preferred option is to form a mutant Bmpr1a sequence that is a truncated sequence, which is a shortened sequence that encodes a nonfunctional Bmpr1a receptor polypeptide molecule. It is most preferred to knock-out Exon 2 of the sequence, resulting in a truncated nonfunctional Bmpr1a gene sequence, such as SEQ ID NO 2. As such, a deletion mutation may be made directly in the sequence.

Alternatively, if a conditional mutant is to be formed, the Bmpr1a nucleic acid sequence should be such that it is fully functional throughout the development of the organism until steps are taken to inactivate the nucleotide sequence. Inactivation occurs once the organism has sufficiently developed. Conditional mutant formation is accomplished by placing nucleotide sequences flanked by recombination sequences into the genome so that the recombination sequence can be later activated. The recombination sequence can be used to cleave a gene or exon from the genome. Preferably, a pair of recombination fragments is used. This can be accomplished by placing the sequence in a vector that places recombination sites on either end of the desired nucleotide sequence. The recombination sites are substituted with the nucleotide sequence into the organism, with the recombination sites activated at a later time.

Next, either the conditional recombination sequence or mutant sequence is inserted into a vector. The vector for forming the conditional mutant will include the targeted Bmpr1a nucleic acid sequence, preferably flanked by recombination sites for the conditional sequence. The conditional vector is structured such that the targeted, recombination-site flanked gene or nucleotide sequence will be cut from the genome to form a knock-out mutant.

Alternatively, a mutated nucleotide sequences, or Bmpr1a gene, or sequence in a vector is directly substituted for the Wt in a cell to render a Bmpr1a gene nonfunctional. Substitution, deletion, loss of function, and frame shift mutations are examples of mutant Bmpr1a sequences that result in the nonfunctional gene. Regardless of the mutant formed, the Wt nucleotide sequence, including the Bmpr1a gene sequence found in a selected host organism, will be substantially eliminated or made nonfunctional through insertion of the vector's mutant nucleic acid sequence. SEQ ID NO 2 is an example of a mutated Bmpr1a sequence that can be used in a recombination vector to obtain the Bmpr1a mutant organism. The truncated, inactive mutant Bmpr1a polypeptide of SEQ ID NO 5 is encoded by the truncated mutant nucleic acid sequence of SEQ ID NO 2.

In determining whether a polypeptide or polynucleotide is substantially homologous to a polypeptide or nucleotide suitable for use in the current invention, sequence similarity may be determined by conventional algorithms, which typically allow introduction of a small number of gaps in order to achieve the best fit. In particular, “percent homology” of two polypeptides or two nucleic acid sequences is determined using the algorithm of Karlin and Altschul (Proc. Natl. Acad. Sci. USA 87:2264-2268, 1993). Such an algorithm is incorporated into the NBLAST and XBLAST programs of Altschul et al. (J. Mol. Biol. 215:403-410, 1990). BLAST nucleotide searches may be performed with the NBLAST program to obtain nucleotide sequences homologous to a nucleic acid molecule of the invention. Equally, BLAST protein searches may be performed with the XBLAST program to obtain amino acid sequences that are homologous to a polypeptide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., XBLAST and NBLAST) are employed. See http://www.ncbi.nlm.nih.gov for more details.

In either mutant, any of a variety of vectors may be used. Formation of the vector follows standard and known procedures and protocols. Suitable vectors include expression vectors, fusion vectors, gene therapy vectors, two-hybrid vectors, reverse two-hybrid vectors, sequencing vectors, and cloning vectors. Vectors are formed from both the isolated nucleic acid sequences and the mutant versions of the isolated nucleic acid sequences.

Eukaryotic and prokaryotic vectors may be used. Specific eukaryotic vectors that may be used include MSCV, Harvey murine sarcoma virus, pFastBac, pFastBac HT, pFastBac DUAL, pSFV, pTet-Splice, pEUK-C1, pPUR, pMAM, pMAMneo, pBI101, pBI121, pDR2, pCMVEBNA, YACneo, pSVK3, pSVL, pMSG, pCH110, pKK232-8, p3′SS, pBlueBacIII, pCDM8, pcDNA1, pZeoSV, pcDNA3, pREP4, pCEP4, and pEBVHis vectors. The MSCV or Harvey murine sarcoma virus is preferred. Prokaryotic vectors that can be used in the present invention include pET, pET28, pcDNA3.1/V5-His-TOPO, pCS2+, pcDNA II, pSL301, pSE280, pSE380, pSE420, pTrcHis, pRSET, pGEMEX-1, pGEMEX-2, pTrc99A, pKK223-3, pGEX, pEZZ18, pRIT2T, pMC1871, pKK233-2, pKK38801, and pProEx-HT vectors.

A variety of selectable markers may be included with the vector. Available markers include antibiotic resistance genes, a tRNA gene, auxotrophic genes, toxic genes, phenotypic markers, colorimetric markers, antisense oligonucleotides, restriction endonuclease, enzyme cleavage sites, protein binding sites, and immunoglobulin binding sites. Specific selectable markers available include LacZ, neo, Fc, DIG, Myc, and FLAG.

The conditional vector will be used to transfect any of a variety of cells. It is preferred to transfect ES cells, with the recombination sequence ultimately present in ISC. Typically, the ES cells will be transplanted into the uterus of an adoptive host mother, so that an embryo can gestate from the ES cells. The vector could also be used to transfect ISC in a mature organism, such as an embryo. The particular type of cell to be transfected will influence the vector selected. Also, the cells to be transfected can be grown in vivo or in vitro. The mutant sequence can be used to transfect ISCs or related intestinal cells present in an embryo or more mature organisms.

The conditional vector will include recombination sites that cause insertion of a conditional knock-out mutation (Bmpr1a^{fx/fx}, for example) or a mutant, wherein Bmpr1a is rendered nonfunctional. Formation of a conditional transgenic Bmpr1a knock-out organism is preferred. This can be achieved by the knock-in of a Cre or Flp recombinase site, or a Cre-Fre site combination thereof, into a specific Bmpr1a gene locus or loci. The expression of Cre or Flp recombinase will be under the control of the endogenous locus in a tissue-specific, time-dependent manner. The temporal/spatial-restricted Cre/Flp expression line will lead to a conditional or selective deletion of the target gene (e.g., Bmpr1a) when crossed with an organism in which LoxP or FRT recombination sites flank the target gene. Preferably, LacZ and GFP markers, flanked by LoxP or FRT recombination sites, may be utilized to determine the efficiency of recombination of the target gene. A combination of the Cre/LoxP and Flp/FRT systems will also allow selective and simultaneous deletion of the two gene loci of interest. Other alternative recombination systems and marker systems, however, can be devised and used as known in the art.

The two functional units required for in vivo targeted conditional DNA deletion of the Bmpr1a receptor gene in the Cre-LoxP organism system are: (1) expression of the PI Cre recombinase gene, often induced by a cell-specific or regulated promoter; and (2) at least one integrated DNA target gene segment that is flanked by LoxP, a 34 bp P1 DNA sequence. The LoxP-flanked target DNA is said to be “floxed.” The Cre/LoxP system is a tool for conditional tissue-specific and time-specific post-natal knock-out of selected target genes (e.g., Bmpr1a), which cannot be investigated in conventional gene knock-out animals, such as mice, because of the nonfunctional target gene's early embryonic lethality.

Thus, a Bmpr1a gene, or other nucleotide sequence, is isolated, and a modified nucleotide sequence or Bmpr1a gene is made by insertion of Lox recombination sites and marker sites into the gene. A Bmpr1a vector is made by insertion of the modified Bmpr1a gene into a vector. An ES cell is then transfected with the Bmpr1a vector to form a Bmpr1a embryonic stem cell. The Bmpr1a embryonic stem cell is implanted into a host uterus to form a Bmpr1a^{fx/fx} organism. The foregoing method can be modified, wherein Bmpr1a vector formation involves insertion of Lox recombination sites flanking Exon 2 of the Bmpr1a gene and insertion of marker sites into the vector's genomic sequence. Another method of modification utilizes a mutant Bmpr1a nucleic acid sequence, which can be administered to the ES cell by methods including, but not limited to, electroporation, microinjection, micro-vessel transfer, particle bombardment, and liposome mediated transfer.

Any of a variety of host cells, including eukaryotic and prokaryotic cells, can be transfected with the vectors previously mentioned. Prokaryotic host cells include Gram-negative and Gram-positive bacteria that may be transfected with any of the variety of the vectors previously mentioned. Available bacteria include Escherichia, Salmonella, Proteus, Clostridium, Klebsiella, Bacillus, Streptomyces, and Pseudomonas. A preferred Gram-negative bacterium is Escherichia coli.

Eukaryotic vectors can be used to transfect eukaryotic host cells including mammalian, amphibian, or insect cells; examples include human, mouse, and frog cells. The preferred process includes transfecting an embryonic stem cell of a selected species with the vector. The transfected embryonic stem cell is then transplanted into an adopted host mother. The embryonic stem cell will gestate to an embryo followed by birth of a conditional mutant organism. Thus, mutant offspring are formed, such as a Bmpr1a^{fx/fx} mutant organism. Specific conditionally active mutants include ISCs.

Typically, two organism (mouse, for example) lines are required for formation of a conditional gene deletion organism: a conventional transgenic line with, for example, Cre-targeted to a specific tissue or cell type, and a strain that embodies a target gene (endogenous gene or transgene) flanked by two recombination (LoxP, for example) sites in a direct orientation (“floxed gene”). When the target gene is the Bmpr1a gene, recombination occurs by excision and, consequently, inactivation of the floxed Bmpr1a target gene. Since recombination and Bmpr1a gene excision occurs only in those cells expressing Cre recombinase, the Bmpr1a target gene remains active in all cells and tissues that do not express Cre recombinase. Gene excision is induced by a recombination activator, such as PolyI:C or interferon, which in turn triggers Cre recombinase expression. The recombination activator is preferably injected postnatally to ensure organism survival. Most preferably the recombination activator is injected at 0, 1, 2, or 20 days after birth, or anytime thereafter. Cre and FLP recombinase are exemplary recombinases that may be used. Cre recombinase is used to cleave Lox sites flanking the Bmpr1a gene, such as LoxP and LoxC2 sites. Alternatively, FLP recombinase can be used with FRT recombination sites flanking the Bmpr1a gene.

For example, Mx1-Cre⁺ and Bmpr1a^{fx/fx} mice progeny are crossed to form a conditional mouse mutant Mx1-Cre⁺Bmpr1a^{fx/fx}. This organism can be conditionally mutated after birth to cause formation of tumors and polyps in the colon and small intestine. Once activated and mutated, an inactive Bmpr1a receptor polypeptide is expressed. An inactive ISC containing a truncated Bmpr1a receptor polypeptide is formed, wherein BMP interaction is blocked. Any of a variety of recombination site-flanked Bmpr1a nucleic acid sequences can be knocked out and expressed. Flanking Bmpr1a recombination sites included in the present invention are Lox, LoxP, and FRT sites.

The knock-out organism permits conditional excision of the target Bmpr1a gene upon the injection of a recombination activator into the organism. The knock-out animal may be a pre-recombination or post-recombination animal, where the pre-recombination animal is the Bmpr1a mutant animal prior to injection of the recombination activator and the post-recombination animal is the Bmpr1a mutant animal after injection of the activator.

Bmpr1a^{fx/fx} and Bmpr1a^{fx/fx} Z/EG knock-out mutant organisms are useful in characterizing a mutant phenotypic change in an intestinal cell in vivo in the organism. The characterized phenotypic change can be the presence of increased ISC population numbers, differentiation change, intestinal polyposis, crypt fission, symmetrical cell division, reduced apoptosis, and/or intestinal tumorigenesis.

A pre-recombination Mx1-Cre⁺Bmpr1a^{fx/fx} Z/EG knock-out mutant organism for use in studying an intestinal cell can be formed. The Mx1-Cre Lox Bmpr1a^{fx/fx} organism, obtained utilizing the previously described method, is crossed with a Z/EG organism to form a pre-excision hybrid Mx1-Cre Lox Bmpr1a^{fx/fx} Z/EG organism. Finally, a recombination activator is administered to the hybrid Mx1-Cre Lox Bmpr1a^{fx/fx} organism crossed with a Z/EG organism to induce Cre-mediated Lox site-directed intracellular Bmpr1a gene recombination. The post-recombination Mx1-Cre⁺Bmpr1a^{fx/fx} Z/EG knock-out mutant organism can be utilized to assess the efficacy of the recombination procedure in yielding intestinal cells with the excised Bmpr1a gene encoding the inactive Bmpr1a receptor. The efficiency of the Bmpr1a gene recombination process is monitored by the detection of LacZ or GFP gene marker expression in intestinal tissue and cells.

Operative recombination activators can include PolyI:C, interferon, or other interferon inducers. PolyI:C is a preferred recombination activator. The recombination activator induces Cre recombinase expression, which in turn results in excision of the Lox-flanked Bmpr1a nucleic acid sequence in cells of the mutant Bmpr1a organism. Preferably, Exon 2 of Bmpr1a is excised, rendering the Bmpr1a gene nonfunctional.

In the intestinal tissue of the transfected animal, the resultant mutant Bmpr1a intestinal cell contains a conditional mutant Bmpr1a gene that can encode an inactive Bmpr1a polypeptide. Instead of Bmpr1a, other nucleotide sequences can be selected for knock-out or mutation. Alternatively, the cells can be mutagenized and nonconditional. The mutant intestinal cells include ISC, progenitor, self-renewing ISC, mucosal progenitor, columnar progenitor, endocrine, paneth, goblet, and enterocyte cells. The mutant intestinal cell may be made in vivo or in vitro by methods such as knock-out organism formation, vector transfection, micro-vessel transfer, biolistic particle delivery, liposome-mediated transfer, electroporation, or microinjection of the Bmpr1a mutant gene or other nucleotide sequence, such as BMP mutant. The mutant intestinal cell is situated in the villus or crypt regions. The intestinal tissue or cells can be isolated and transfected.

A mutant intestinal cell having an inactive Bmpr1a receptor polypeptide can be formed by activating the recombinase in the knock-out organism as herein described. The mutant intestinal cell's Bmpr1a binding to BMP is substantially inhibited. In particular, the mutant intestinal cell can include the inactive Bmpr1a receptor polypeptide that is truncated or a shortened Bmpr1a receptor polypeptide, such as the shortened Bmpr1a receptor polypeptide of SEQ ID NO 5. This truncated Bmpr1a receptor polypeptide is encoded by a truncated, nonfunctional Bmpr1a gene (SEQ ID NO 2) in which Exon 2 has been excised (SEQ ID NO 3). This mutant Bmpr1a intestinal cell either possesses an inactive Bmpr1a polypeptide or lacks the Bmpr1a polypeptide completely.

Because the mutational changes are typically clonal and expressed throughout the crypt and villus, the mutant intestinal cell, including the Bmpr1a mutant, includes resting, self-renewing, proliferating, transient amplifying, differentiating, and apoptotic cells. In particular, it includes mesenchymal, mucosal, mucosal progenitor, columnar, columnar progenitor, goblet, paneth, tumor, and polyp cells. The mutant intestinal cell can be located in the knock-out organism or in isolated intestinal tissue placed in vitro. The Bmpr1a mutant intestinal cell exhibits asymmetrical and symmetrical division in the proliferation zone.

An isolated Bmpr1a antisense fragment or antisense oligonucleotide that exists intracellularly can be used to influence ISC proliferation and development, so that the antisense fragment induces ISC proliferation by inhibiting translation of Bmpr1a receptor polypeptide (SEQ ID NO 4). This can cause increased proliferation of mucosal progenitors and a decrease in columnar progenitors. The antisense sequence will also cause an increase in ISC self-renewal, leading to crypt fission due to symmetrical division of the stem cells. The antisense fragment can be inserted into the ISC or other intestinal cells by methods including, but not limited to, electroporation, transfection, microinjection, micro-vessel transfer, particle bombardment, biolistic particle delivery, and liposome mediated transfer. The antisense fragment can also be directed to BMP or PTEN pathway members. The isolated Bmpr1a antisense fragment can be synthesized and multiple copies generated in vitro using a sense template, as is known in the art. An example of an antisense fragment is RNAi.

The Noggin protein or polypeptide can be used to competitively bind to Bmpr1a receptor which, in turn, affects ISC expansion and commitment. In particular, an isolated Noggin activator (Noggin polypeptide), or fragments thereof can be used to block BMP and cause increased ISC self-renewal. The Noggin activator acts to induce ISC proliferation in vitro by inhibiting BMP binding to the Bmpr1a receptor (SEQ ID NO 4). Noggin's binding affinity for the Bmpr1a receptor can be greater than BMP's affinity for the receptor. Noggin can be used in cells, tissue, or organisms, the same as the conditional or mutant Bmpr1a knock-out. Increased amounts of Noggin can be expressed by using a vector. The vector will typically locate in the cytoplasm and “flood” the cell with the Noggin polypeptide. Another option is to contact the cell, tissue, or organism with increased amounts of the Noggin polypeptide. Wt intestinal tissue can be exposed to a stem cell activator, such as Noggin, and cultivated in culture medium in vitro. An example of a stem cell activator is Noggin at a concentration in medium of between 10 ng/ml and 200 ng/ml. The Noggin can be contained in beads, particles, or liposomes. Preferably, Noggin-beads are injected into the intestinal tissue, placing Noggin in contact with the ISCs and other intestinal cells. Alternative activators could be used, such as members of the PTEN pathway. The alternative activators can also be provided via beads, particles, or liposomes.

An antibody to a gene product or protein, particularly BMP or Bmpr1a, can be used to generate phenotypic changes in a selected host organism. The antibody can be designed to attack the Bmpr1a or BMP polypeptide. Use of such an antibody will prevent the functioning of the Bmpr1a or BMP polypeptide and, thus, result in increased proliferation, self-renewal, mutant differentiation, and increased apoptosis in vivo or in vitro. An antibody to the Wt or mutant Bmpr1a polypeptide also will be used to detect and monitor the presence of Wt or mutant Bmpr1a in intestinal cells. Thus, isolated antibodies, such as anti-Bmpr1a antibody, anti-BMP antibody, and fragments thereof, where the antibody, acting as an intestinal stem cell (ISC) activator, induces ISC proliferation in vitro by inhibiting BMP binding to Bmpr1a receptor can be used. Anti-Bmpr1a antibodies and anti-BMP antibodies are made, isolated, and administered to an ISC or intestinal cell population in vitro to attack BMP. Binding of the Bmpr1a receptor to the BMP polypeptide is inhibited by the binding of either the anti-Bmpr1a antibody or anti-BMP antibody to the ISC population. This will cause the ISC population to be expanded in vitro. Administration of the isolated antibodies to the ISC population may occur by injection, transfection, particle-mediated delivery, liposome encapsulation, diffusion, or micro-vessel encapsulation. Antibodies can be obtained by polyclonal or monoclonal methodologies known to those in the art.

As discussed, an alternative to forming a Bmpr1a knock-out is to mutagenize genes related to the BMP and Bmpr1a pathway. This can be accomplished by forming a vector having a promoter and a PTEN pathway gene. The PTEN gene can be mutagenized in advance, or the vector can be used to form a knock-out. The PTEN pathway genes include Noggin, PTEN, AKT, GSK-3, cyclin D1, Tert, PI3K, SMAD1,5,8, p27, and mutant genes related thereto. PTEN pathway component effects occur downstream from the BMP-Bmpr1a receptor triggering event taking place at the intestinal cell membrane. By activating these PTEN pathway genes, effects similar to the mutagenesis of the Bmpr1a gene can be achieved, since both routes lead to the diminution of effects of BMP signaling. The PTEN pathway vector can be utilized in vitro or in vivo. Preferably, the PTEN pathway vector can be used to induce intestinal cell proliferation, differentiation, or apoptosis. Like before, these can be conditional or actual mutants. Also, cells, tissue, or organisms can be transfected.

Prokaryotic organisms, such as bacterial species, containing a prokaryotic PTEN pathway vector can be developed. The prokaryote will include Wt or mutant PTEN pathway nucleotide sequence.

An in vitro intestinal stem cell cultivation system is developed, wherein an activated intestinal stem cell population or intestinal cell population self-renews, proliferates, has mutant differentiation, and reduced apoptosis. The cultivation system includes an isolated intestinal tissue, a culture medium, and an isolated stem cell activator. The activator operatively attaches to at least one stem cell, or intestinal cell, in the population. The activator can be a mutant Bmpr1a receptor polypeptide, a mutant Bmpr1a receptor nucleotide sequence, an anti-Bmpr1a antibody, a Wt Bmpr1a receptor antisense sequence, a Noggin polypeptide, a BMP polypeptide, a PTEN family polypeptide, an antisense fragment, or a fragment thereof. The intestinal tissue can be of mammalian origin. In particular, human tissue can be isolated with the cells, then mutagenized to prevent BMP and Bmpr1a interaction. Inhibition of BMP should cause tumor and polyp formation in vitro. Additionally, the ISCs can be studied.

An exemplary in vitro intestinal tissue cultivation system causes ISC population proliferation in response to a Noggin activator. Other activators, such as anti-BMP and anti-Bmpr1a antibodies, anti-BMP antibodies, or fragments thereof may be used. This cultivation system contains isolated intestinal tissue, culture medium, and an effective amount of isolated Noggin polypeptides, or other activators. Alternatively, instead of tissue, the cultivation system can contain an isolated intestinal stem cell population comprising at least 10⁴ cells. The intestinal stem cell population can be isolated by FACS methods using antibodies directed against ISC-associated antigens, such as anti-Bmpr1a receptor polypeptide. Isolated Noggin polypeptides, which include truncated polypeptides or Noggin fragments, are contacted in vitro with the Bmpr1a cell receptors. The Bmpr1a receptor binding to BMP is substantially inhibited by Noggin.

The activator can be placed in operative contact with the intestinal stem cell population by means of an activator insertion device. Activator insertion devices can be injection, diffusion, particle-mediated, micro-vessel encapsulation, or liposome encapsulation devices. An in vitro mutant Bmpr1a intestinal stem cell cultivation system results, wherein a mutant intestinal stem cell population proliferates, having the following: an isolated mutant Bmpr1a intestinal stem cell population comprising an inactive Bmpr1a receptor and culture medium. Bmpr1a gene mutations in the mutant intestinal stem cell can be a frame shift, substitution, loss of function, or deletion mutation.

A final tissue system can be developed by isolating an intestinal tissue sample, that is then placed in media. The tissue is isolated from the digestive tract, and will include the crypt/villus region, as well as ISCs. Vectors, previously discussed, can be used to transfect the cells. The tissue cells will be allowed to proliferate, with the results of the mutants then observed.

As a result of the above, a variety of methods can be practiced, which influence intestinal stem cells and differentiated intestinal cells. Methods for causing increased self-renewal can be practiced. One method includes preventing BMP from binding to Bmpr1a. This can be accomplished by a knock-out of BMP or Bmpr1a. An alternative approach involves phosphorylating AKT to form a P-AKT, which can be done using an inhibitor, such as Ly294002. This can also be accomplished by blocking BMP PTEN interaction to form P-PTEN. Also, 14-3-3ζ and AKT can be used to control self-renewal of ISC and potential stem cells in other tissues.

The pathway illustrated in FIG. 18 can be used to control self-renewal, proliferation, differentiation, and apoptosis. The pathway can be controlled by a number of proteins.

Targets for control of the intestinal cells are provided. The discussed target proteins can be turned on or off to control intestinal cell fate.

The previously discussed resultant mouse model can be used for studying human JPS. Inactivation of the Bmpr1a receptor causes formation of polyps throughout the intestinal tract. The intestinal cell fate lineage commitment is studied in comparison to columnar cell fate lineage commitment. Intestinal cells studied are goblet, paneth, mucin-producing, enterocyte, tumorous, and polyp cells using previously described cell markers.

Various proteins can be used to mark particular types of cells. Examples of protein markers used to identify an ISC population having increased self-renewal are P-AKT, 14-3-3ζ, Nd P-PTEN. Increased proliferation, which leads to crypt fission and polyposis, can be identified by increased P-PTEN, P-AKT, β-catenin, and tert. Abnormal differentiation, which results in increased mucosal progenitor, paneth, and goblet cells, is also identified by increased P-PTEN, P-AKT, β-catenin, and tert. Increased apoptosis is identified by increased P-BAD.

The BMP pathway influences self-renewal, differentiation, and apoptosis in ISC and intestinal cells, and is illustrated in FIG. 18. The pathway can be used to control cells in vivo or in vitro. A population of ISCs with increased self-renewal are identified by various markers, including P-PTEN⁺, P-AKT⁺, nuclear accumulated β-catenin, 14-3-3 ζ, and Tert⁺. A population of transient amplifying progenitors which are proliferating are identified by markers Ki67+, Brd-U⁺, and P-PTEN⁺. The markers for identifying inhibited apoptosis in intestinal cells are BAD, 14-3-3ζ, and Tunel. Thus, a group of markers for determining whether intestinal cells are mutagenized, are identified. The markers for use in identifying the various types of cells include Ki67, P-PTEN, PTEN, AKT, P-AKT, Tert, β-catenin, P-Smad1,5,8, BMP, Noggin, Bmpr1a, BAD, P-BAD, 14-3-3ζ, and combinations thereof.

A variety of kits can be formed either from the mutant or Wt polypeptides or the nucleic acid sequences associated with intestinal tissue or cells. Kits are described for detection of mutant or variant forms of the aforementioned nucleic acid molecules, detection of expressed polypeptides or proteins, and measurement of corresponding levels of protein expression. Kits can detect the presence or absence of mutants and non-mutants of the nucleic acid molecules, and their expressed amino acid sequences or polypeptide molecules. The kit will preferably have a container and either at least one nucleic acid molecule, or a polypeptide molecule, which includes any of the aforementioned sequences.

A kit will be formed with a container and a Bmpr1a polypeptide molecule. The kit will detect either a mutant or Wt Bmpr1a polypeptide or nucleic acid molecule in intestinal tissue or cells. Specifically, the kit will be used to detect the presence of a mutant Bmpr1a receptor, gene, or polypeptide. The kit will also detect a mutant ISC containing an inactive Bmpr1a receptor or gene. Kits for detection and quantitation of the presence in intestinal cells of markers such as Bmpr1a, BMP, Noggin, PTEN, P-PTEN, AKT, P-AKT, Tert, β-catenin, Ki67, p27, Smad1,5,8, tubulin, Chromgrin A, BAD, P-BAD, FAK, and 14-3-3ζ polypeptide and nucleic acid markers will be formed. These kits can be used for detection and quantitation of markers associated with intestinal cell activation, proliferation, differentiation, apoptosis, polyposis, and tumor formation. Specifically, immunodiagnostics and nucleic acid probe kits for mutant Bmpr1a intestinal cell expression of the foregoing marker nucleic acid sequence and polypeptide markers will be made and used. In addition, the present invention includes diagnostic methods and kits for the prediction and assessment of intestinal polyposis and tumorigenesis. These foregoing kits may be used either in vitro or in vivo.

In summary, hybridization methodology and kits for the detection, identification, and quantification of Bmpr1a-associated nucleic acid sequences in cells are set forth herein. Using these methods, Bmpr1a Wt and mutant nucleic acid sequences can be identified, characterized, and quantified. In addition, kits may be produced utilizing Bmpr1a-derived nucleic acid molecule standards, antibodies, and kit components as previously described.

Cycle-dependent expression of Noggin regulates BMP activity and, in turn, forms activity gradients along the physical length of the villus axis. Differentially localized BMP activity, which is produced by mesenchymal cells and regulated by Noggin interaction with BMP-receptor type IA (Bmpr1a), defines intestinal architectural zones in which ISCs undergo sequential developmental process: self-renewal, proliferation, differentiation, and apoptosis. Intestinal stem cells are prevented from receiving a BMP signal by inactivation of the Bmpr1a receptor in the Bmpr1a mutant mouse. This Bmpr1a receptor inactivation causes phenotypic expansion in the population of ISCs, impaired differentiation, and resistance to apoptosis. In addition, murine polyposis, similar to JPS in humans is induced.

BMP functions as a regulatory restriction signal in vivo and in vitro to the ISCs through the regulation of PTEN pathway activity, which in turn control the activities of PI3K-AKT-GSK3β, and β-catenin. Blocking the BMP signal in the Bmpr1a mutant causes PTEN pathway activation through PTEN phosphorylation (PTEN→P-PTEN). P-PTEN conversion, in turn, leads to activation of AKT. As such, BMP signal blockage in the Bmpr1a mutant organism, leads to increased self-renewal in ISCs, through PTEN conversion into the phosphorylated form and activation of the PI3K/AKT pathway via activated AKT. This AKT activation initiates stem cell self-renewal by activating Telomerase. In addition, apoptosis is suppressed. The effect of BMP signaling on ISC self-renewal, differentiation, apoptosis, symmetry of cell division, and tumorigenesis is depicted diagramatically in FIGS. 11A-11D. In the fundamental BMP signaling system in Wt animals, BMP bound to the Bmpr1a receptor on the ISC prevents ISC self-renewal by inhibition of the phosphorylation of PTEN. BMP blockage also impairs differentiation because of unbalanced lineage commitment. Additionally, tumor formation occurs in Bmpr1a mutants, with crypt fission due to stem cell division, resulting in an increase in ISC number. The Bmpr1a mutant also exhibits BAD signal blockage, resulting in reduced apoptosis at the tips of the villi.

Noggin activates ISCs in Wt intestine by temporally overriding the BMP signal. Noggin competitively inhibits BMP binding to Bmpr1a cell surface receptor sites. In the presence of Noggin, BMP-mediated inhibition of ISCs is released to ultimately permit proliferation and self-renewal. Proliferation and self-renewal occur at the base region of the villi. Upon Noggin binding to Bmpr1a receptor, p27^{Kip} activity is first reduced, and ISC division is initiated. In the differentiation zone of Wt mice, Noggin binding to the Bmpr1a receptor sites results in increased cell commitment to mucosal cell lineages, evidenced by increased numbers of goblet and paneth cells. Fewer enterocytes will also be observed.

Activated AKT enhances self-renewal of ISCs through two functional routes of action: 1) maintenance of the proliferation potential by Telomerase activation and β-catenin relocation, and 2) provision of a cell survival signal through inhibition of BAD (BAD→P-BAD conversion) and other pro-apoptotic factors.

A switch from entirely asymmetric to randomized symmetric and asymmetric ISC division in the mutants is shown in FIG. 11C. A model of the molecular mechanisms causing tumor formation in the Bmpr1a mutant intestines is illustrated in FIG. 11C. Loss of PTEN activity and increase in AKT activity, resulting from inhibition of the BMP signal, led to an increase in both stem cell self-renewal and in the ISC number.

In the proliferation zone, non-expression of Bmpr1a in mutant mice resulted in no manifest BMP-mediated inhibition. For this reason, stem cells underwent proliferation. An increase in proliferation of progenitor cells was found in the mutant tumor region enriched with multiple crypts, indicated in FIG. 11D, where differentiation was partially inhibited.

The highest level of BMP activity is found in the apoptotic zone, with BMP induced cell apoptosis correlating with increasing the BAD activity. The mutant cells in the apoptotic zone are resistant to apoptosis due to loss of BAD signaling, resulting from inactivation of Bmpr1a.

The murine Bmpr1a conditional inactivation line provides a novel animal model for investigation of the molecular mechanisms that cause JPS and tumorgenesis in humans. Furthermore, elucidation of the pathways that play a role in the etiology of JPS, such as BMP/PTEN/PI3K/AKT/Tert or BAD, will potentially generate molecular biological tools for clinical applicability for the treatment and diagnosis of intestinal cancer and disease.

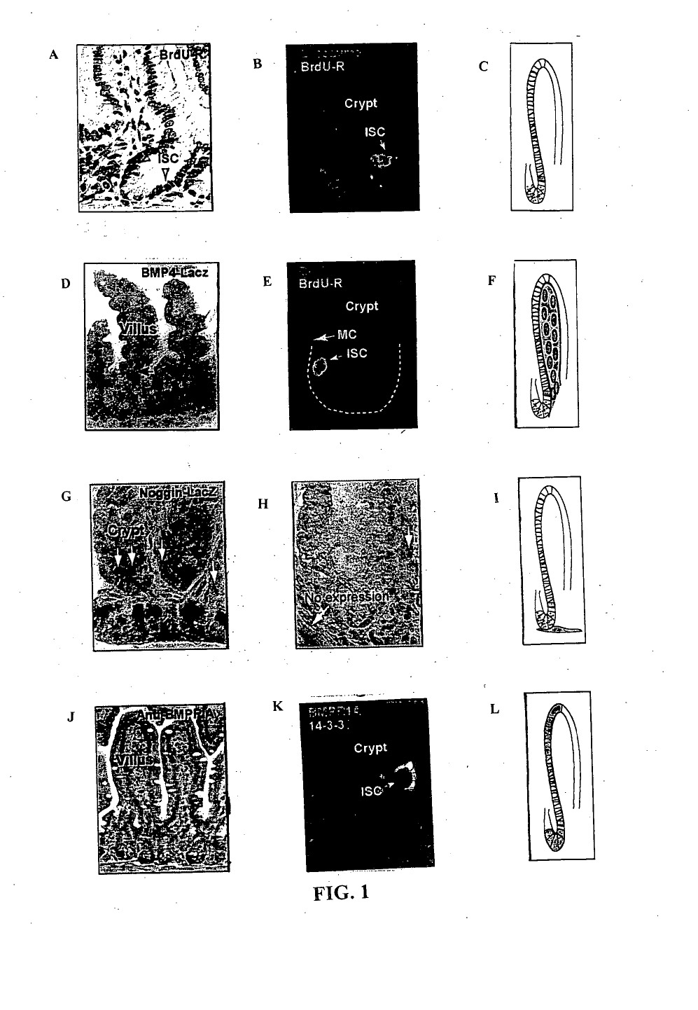

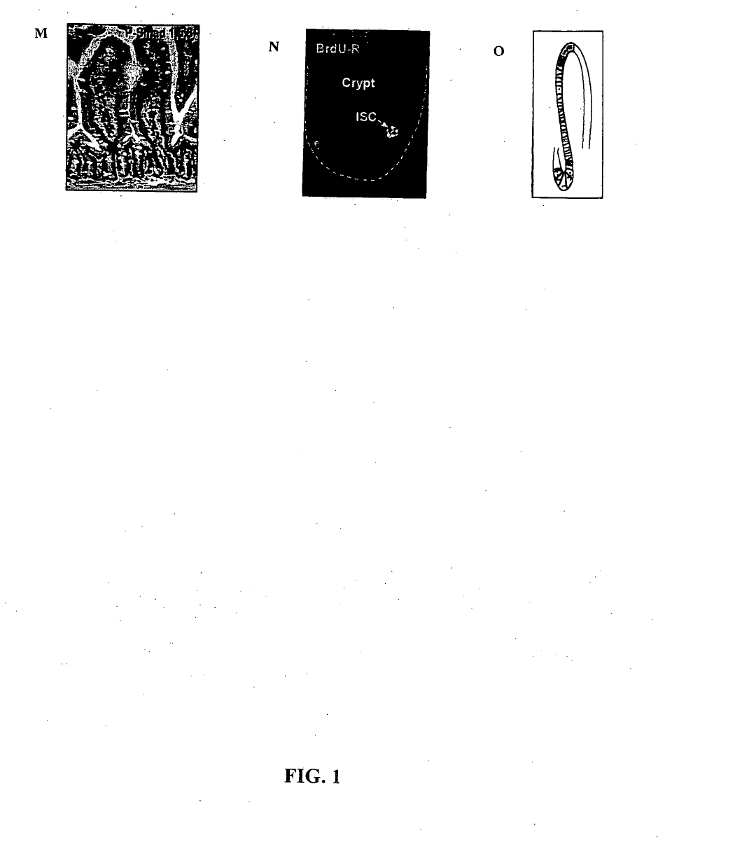

Significantly, it is established herein that the BMP signal controlled the ISC number by restricting activation and expansion of stem cells in homeostasis and regeneration. The Noggin signal overrides the BMP activity, which causes a cascade of the PTEN-PI3K-AKT-GSK3β pathway. Noggin interaction with the Bmpr1a receptor on ISCs results in the translocation of β-catenin from the cytoplasm into the nucleus of the arrested stem cell, thereby activating stem cell division. Bmpr1a receptor inactivation results in blocking intestinal epithelial cells from sensing the BMP signal which in turn generates an increase in the number of long-term (arrested) ISCs, impaired differentiation and resistance to apoptosis, eventually leading to the formation of profuse intestinal polyps and tumors. The BMP signal distribution pattern, which co-existed with a Noggin-dependent activity gradient along the intestinal villus axis, was determined to play a critical role in the control of the number of the intestinal stem cells by restricting activation and expansion of intestinal stem cells. Thus, the BMP signal, with differentially localized activities, defined specified zones within the intestinal villi, as shown in FIG. 1F, in which ISCs proceed through self-renewal, proliferation, differentiation, and apoptosis.

The pathways provide targets, which can be used to design drugs and small molecules for treatment of JPS and other intestinal polyps and tumors. The pathway provides targets for the treatment of polyposis.

The following definitions define terms used herein:

Activated mutant is a post-recombination organism, tissue, or cell wherein the mutant is obtained by injection of a recombination activator into a conditional mutant organism, tissue, or cell to induce a mutation event that results in inactivation of the targeted gene. For example, an activated Bmpr1a mutant organism is a post-excision organism which resulted from PolyI:C injection of a conditional Bmpr1a mutant organism to yield a nonfunctional Bmpr1a gene.

An activator is a molecule that can induce proliferation, self-renewal, cell division, or differentiation in a cell. The activator may optionally induce polyposis or apoptosis in a cell. An intestinal stem cell activator generally induces proliferation or cell division.

Allele is a shorthand form for allelomorph, which is one of a series of possible alternative forms for a given gene differing in the DNA sequence and affecting the functioning of a single product.

An amino acid (aminocarboxylic acid) is a component of proteins and peptides. All amino acids contain a central carbon atom to which an amino group, a carboxyl group, and a hydrogen atom are attached. Joining together of amino acids forms polypeptides. Polypeptides are molecules containing up to 1000 amino acids. Proteins are polypeptide polymers containing 50 or more amino acids.

An antigen (Ag) is any molecule that can bind specifically to an antibody (Ab). Ags can stimulate the formation of Abs. Each Ab molecule has a unique Ag binding pocket that enables it to bind specifically to its corresponding antigen. Abs may be used in conjunction with labels (e.g., enzyme, fluorescence, radioactive) in histological analysis of the presence and distribution of marker Ags. Abs may also be used to purify or separate cell populations bearing marker Ags through methods, including fluorescence activated cell sorter (FACS) technologies. Abs that bind to cell surface receptor Ags can inhibit receptor-specific binding to other molecules to influence cellular function. Abs are often produced in vivo by B cells and plasma cells in response to infection or immunization, bind to and neutralize pathogens, or prepare them for uptake and destruction by phagocytes. Abs may also be produced in vitro by cultivation of plasma cells, B cells or by utilization of genetic engineering technologies.

BMPs constitute a subfamily of the transforming growth factor type beta (TGF-β) supergene family and play a critical role in modulating mesenchymal differentiation and inducing the processes of cartilage and bone formation. BMPs induce ectopic bone formation and support development of the viscera. Exemplary BMPs include those listed by the NcBI, such as human BMP-3 (osteogenic) precursor (NP001192), mouse BMP-6 (NP031582), mouse BMP-4 (149541), mouse BMP-2 precursor (1345611), human BMP-5 preprotein (NP 066551.1), mouse BMP-6 precursor (1705488), human BMP-6 (NP 001709), mouse BMP-2A (A34201), mouse BMP-4 (461633), and human BMP-7 precursor (4502427).

Bmpr1a receptor, or Bmpr1a, is defined as the bone morphogenetic protein receptor, type 1A. Bmpr1a is a regulator of chondrocyte differentiation, down stream mediator of Indian Hedgehog, TGF-β superfamily, and activin receptor-like kinase 3. Binding a ligand to the receptor induces the formation of a complex in which the Type II BMP receptor (Bmpr1b receptor) phosphorylates and activates the Type I BMP receptor (Bmpr1a receptor). Bmpr1a receptor then propagates the signal by phosphorylating a family of signal transducers, the Smad proteins. The Bmpr1a gene encodes the Bmpr1a receptor. Bmpr1a binds to BMP and Noggin.

Bmpr1a mutant organism is defined as an organism lacking a functional Bmpr1a gene or a conditionally activated Bmpr1a gene that can be rendered nonfunctional, where a nonfunctional Bmpr1a gene is one that encodes an inactive Bmpr1a receptor. An example of such an organism is the Mx1-Cre⁺Bmpr1a^{fx/fx} mutant mouse.

Bmpr1a gene (Bone morphogenetic protein receptor, type 1A gene)(ACVRLK3; ALK3) is any Bmpr1a gene isolated from an organism, including human and mouse Bmpr1a genes, as represented in SEQ ID NOs 8 and 1 respectively. The Bmpr1a gene, also known as Activin A receptor, type II-like kinase 3 is GenBank ID BB616238. Homologs from mammals and other organisms are also included. The Bmpr1a gene encodes a Bmpr1a receptor protein. Human and mouse Bmpr1a polypeptides are SEQ ID NOs. 4 and 7 respectively. The Bmpr1a gene may be obtained from cell line XC131 Protein Accession No. XP_{—}017633. The Bmpr1a gene is located on chromosome, locus 10q22.3 in mice; and the human homolog LOC88582 of Bmpr1a is located on Human Chromosome: ′6. The human Bmpr1a gene is SEQ ID NO 8, which encodes the human Bmpr1a polypeptide, SEQ ID NO 7. The Bmpr1a gene produces a Bmpr1a transmembrane receptor with a small cysteine-rich extracellular region, a juxtamembrane region of phosphorylation, that is glycine and serine rich and a cytoplasmic serine/threonine kinase domain. GenBank ID BB616238 is a full-length enriched adult male testis Mus musculus cDNA clone 4931425I16 5′, mRNA sequence. The Bmpr1a receptor is encoded by 11 exons and spans about 40 kb on chromosome 14. Exon 2 of the murine Wt Bmpr1a gene contains nucleotides 68 through 230 of the gene's coding region, as shown in SEQ ID NO 3. This Bmpr1a nucleic acid sequence encodes a region extending from the 23^{rd} amino acid (glycine) through the 77^{th} amino acid (isoleucine) of the Wt Bmpr1a polypeptide chain, as presented in SEQ ID NO 6. The mutant Bmpr1a gene lacking Exon 2 is exhibited in SEQ ID NO 2, while the truncated mutant Bmpr1a polypeptide is presented in SEQ ID NO 5.

BMPRA_Human Protein—GDB 230245: BMPRA is comprised of 532 amino acids and has a molecular weight of 60,201 daltons. The BMPRA protein functions as a receptor for BMP-2 and BMP-4. BMPRA is highly expressed in skeletal muscle and heterodimerizes with a type-II receptor. It belongs to the ser/thr family of protein kinases in the TGFβ receptor subfamily. Bmpr1a Nucleic Acid—is described in the gene atlas database, which is incorporated by reference. This BMPRA protein is located at gene bank ID No. RB616238, and it can also be found at the NCBI Unigene.

A chimera is an individual composed of a mixture of genetically different cells. By definition, genetically different cells of chimeras are derived from genetically different zygotes.

A conditional mutant is a pre-recombination organism, tissue, or cell wherein injection of a recombination activator into the conditional mutant organism, tissue, or cell induces a mutation event that results in inactivation of the targeted gene, resulting in formation of an activated Bmpr1a mutant organism.

A conditional Bmpr1a mutant knock-out organism can be a pre-recombination or post-recombination Bmpr1a mutant organism. An example of a conditional Bmpr1a mutant knock-out organism is a Mx1-Cre⁺Bmpr1a^{fx/fx} or Mx1-Cre⁺Bmpr1a^{fx/fx} Z/EG organism. The mutant organism may be a mouse. Upon administration of a recombination activator, such as PolyI:C, to the pre-recombination Bmpr1a mutant organism, a post-recombination Bmpr1a mutant organism is formed in which the cells may contain a mutant Bmpr1a nucleic acid sequence. The recombination activator may be administered either prenatally or postnatally to induce Bmpr1a mutation in the cells.

Differentiation occurs when a cell transforms itself into another form. For example, a hematopoietic stem cell (HSC) may differentiate into cells of the lymphoid or myeloid pathways. The HSC might differentiate into lymphocytes, monocytes, polymorphonuclear leukocytes, neutrophils, basophils, or eosinophils. Similarly, an ISC may differentiate into cells of the mucosal or columnar differentiation pathways. An ISC may differentiate into a mucosal progenitor cell, which gives rise to a mucus-secreting goblet cell.

Expression cassette (or DNA cassette) is a DNA sequence that can be inserted into a cell's DNA sequence. The cell in which the expression cassette is inserted can be a prokaryotic or eukaryotic cell. The prokaryotic cell may be a bacterial cell. The expression cassette may include one or more markers, such as Neo and/or LacZ. The cassette may contain stop codons. In particular, a Neo-LacZ cassette is an expression cassette that can be placed in a bacterial artificial chromosome (BAC) for insertion into a cell's DNA sequence. Such expression cassettes can be used in homologous recombination to insert specific DNA sequences into targeted areas in known genes.

A gene is a hereditary unit that has one or more specific effects upon the phenotype of the organism; and the gene can mutate to various allelic forms. The gene is generally comprised of DNA or RNA.

Green fluorescent protein (GFP) is comprised of 238 amino acids and is a spontaneously fluorescent protein isolated from coelenterates, such as the Pacific jellyfish, Aequoria victoria. It transduces, by energy transfer, the blue chemiluminescence of another protein, aequorin, into green fluorescent light. GFP can function as a protein tag to a broad variety of proteins, many of which have been shown to retain native function upon GFP binding. GFP is used as a noninvasive marker in living cells to allow numerous other applications such as a cell lineage tracer, reporter of gene expression and as a potential measure of protein-protein interactions.

Homolog relates to nucleotide or amino acid sequences which have similar sequences and that function in the same way.

A host cell is a cell that receives a foreign biological molecule, including a genetic construct or antibody, such as a vector containing a gene.

A host organism is an organism that receives a foreign biological molecule, including a genetic construct or antibody, such as a vector containing a gene.

Intestinal epithelial stem cell (ISC) is an intestinal stem cell that is distinguishable from progeny daughter stem cells. ISCs can be induced by an activator to undergo proliferation or differentiation. The ISC activator may be produced endogeneously by another intestinal cell, such as a mesenchymal cell. Alternatively, the ISC activator may also be exogeneously administered to the cell. ISCs may be located at the base of the villi, in or adjacent to the crypt region of the small and large intestine.

Intestinal tissue is isolated large or small intestine tissue obtained from an organism, and this tissue possesses villi, lumen, crypts, other intestinal microstructures, or portions thereof. Intestinal tissue can be derived from either Wt or mutant organisms. Intestinal tissue includes intestinal stem cells. Intestinal tissue may be cultivated in vitro or in vivo.

JPS is characterized in intestinal tissue by focal hamartomatous malformations and slightly lobulated lesions with stalks. The polyps enclose abundant cystically dilated glands with normal epithelium, but they have hypertrophic lamina propria and mucosal cysts. In humans, JPS is an autosomal dominant gastrointestinal hamartomatous polyposis syndrome, where patients are at risk for developing gastrointestinal cancers. JPS patients may exhibit mutations in the Bmpr1a, MADH4, or PTEN genes.

Knock-out is an informal term coined for the generation of a mutant organism (generally a mouse) containing a null or inactive allele of a gene under study. Usually the animal is genetically engineered with specified wild-type alleles replaced with mutated ones. Knock-out also refers to the mutant organism or animal. The knock-out process may involve administration of a recombination activator that excises a gene, or portion thereof, to inactivate or “knock out” the gene. The knock-out organism containing the excised gene produces a nonfunctional polypeptide.

A label is a molecule that is used to detect or quantitate a marker associated with a cell or cell type. Labels may be nonisotopic or isotopic. Representative, nonlimiting nonisotopic labels may be fluorescent, enzymatic, luminescent, chemiluminescent, or colorimetric. Exemplary isotopic labels may be H³, C¹⁴, or P³². Enzyme labels may be horseradish peroxidase, alkaline phosphatase, or β-galactosidase labels conjugated to anti-marker antibodies. Such enzyme-antibody labels may be used to visualize markers associated with cells in intestinal or other tissue.

A marker is an indicator that characterizes either a cell type or a cell that exists in a particular state or stage. A stem cell marker is a marker that characterizes a specific cell type that can possess a cell function such as self-renewal, proliferation, differentiation, or apoptosis. The marker may be external or internal to the cell. An external marker may be a cell surface marker. An internal marker may exist in the nucleus or cytoplasm of the cell. Markers can include, but are not limited to polypeptides or nucleic acids derived from Bmpr1a, BMP, Noggin, PTEN, P-PTEN, AKT, PAKT, Tert, β-catenin, Ki67, p27, Smad1,5,8, tubulin, Chromgrin A, BAD, PBAD, FAK, GFP, and LacZ molecules, and mutant molecules thereof. Markers may also be antibodies to the foregoing molecules, and mutants thereof. For example, antibodies to Bmpr1a, BMP, and Noggin can serve as markers that indicate the presence of these respective molecules within cells, on the surface of cells, or otherwise associated with cells. GFP and LacZ marker sites can indicate that recombination occurs in a target gene, such as the Bmpr1a gene.

A mutation is defined as a genotypic or phenotypic variant resulting from a changed or new gene in comparison with the Wt gene. The genotypic mutation may be a frame shift, substitution, loss of function, or deletion mutation, which distinguishes the mutant gene sequence from the Wt gene sequence.

A mutant is an organism bearing a mutant gene that expresses itself in the phenotype of the organism. Mutants may possess either a gene mutation that is a change in a nucleic acid sequence in comparison to Wt, or a gene mutation that results from the elimination or excision of a sequence. In addition polypeptides can be expressed from the mutants.

Noggin is a polypeptide that is an inhibitor of BMPs, and its inhibitory activity is manifested through binding to the Bmpr1a receptor. Noggin is required for embryonic growth and patterning of the neural tube and somite. Noggin is also essential for cartilage morphogenesis and joint formation. Mouse Noggin polypeptide and nucleic acid sequences are SEQ ID NOs 11 and 12, respectively. Human polypeptides and nucleic acid sequences are SEQ ID NOs 9 and 10, respectively.

A nucleic acid or nucleotide sequence is a nucleotide polymer. Nucleic acid also refers to the monomeric units from which DNA or RNA polymers are constructed, wherein the unit consists of a purine or pyrimidine base, a pentose, and a phosphoric acid group.

A nucleotide sequence is a nucleotide polymer, including genes, gene fragments, oligonucleotides, polynucleotides, and other nucleic acid sequences.

Plasmids are double-stranded, closed DNA molecules ranging in size from 1 to 200 kilo-bases. Plasmids are used as vectors for transfecting a host with a nucleic acid molecule.

PolyI:C is an interferon inducer consisting of a synthetic, mismatched double-stranded RNA. The polymer is made of one strand each of polyinosinic acid and polycytidylic acid. PolyI:C is 5′-Inosinic acid homopolymer complexed with 5′-cytidylic acid homopolymer (1:1). PolyI:C's pharmacological action includes antiviral activity.

A polypeptide is an amino acid polymer comprising at least two amino acids.

A post-excision mutant organism is an organism, a targeted gene, or sections thereof, wherein the targeted gene or section has been excised by recombination. The post-excision organism is called a “knock-out” organism. Administration of a recombination activator, such as PolyI:C or interferon, can induce the recombination event resulting in target gene excision. A post-excision Bmpr1a mutant organism is one in which the Bmpr1a gene has been inactivated.

A pre-excision Bmpr1a mutant organism is one that has recombination sites flanking regions of the Bmpr1a gene. The pre-excision organism generally has recombinase-encoded sites that can be induced to express Cre or Flp recombinase, but remain dormant or unexpressed until cells of the organism are exposed to a recombination activator. Administration of the activator to the pre-excision Bmpr1a mutant organism under proper conditions can transform it into a post-excision Bmpr1a mutant organism.

Proliferation occurs when a cell divides and results in progeny cells. Proliferation can occur in the self-renewal or proliferation zones of the intestinal villus. Stem cells may undergo proliferation upon receipt of molecular signals such as those transmitted through Bmpr1a cellular receptor.

PTEN family nucleotide sequence includes, but is not limited to, the following: PTEN, PI3K, AKT, Tert, β-catenin, P27, and BAD nucleic acid sequences, and mutant sequences derived therefrom.

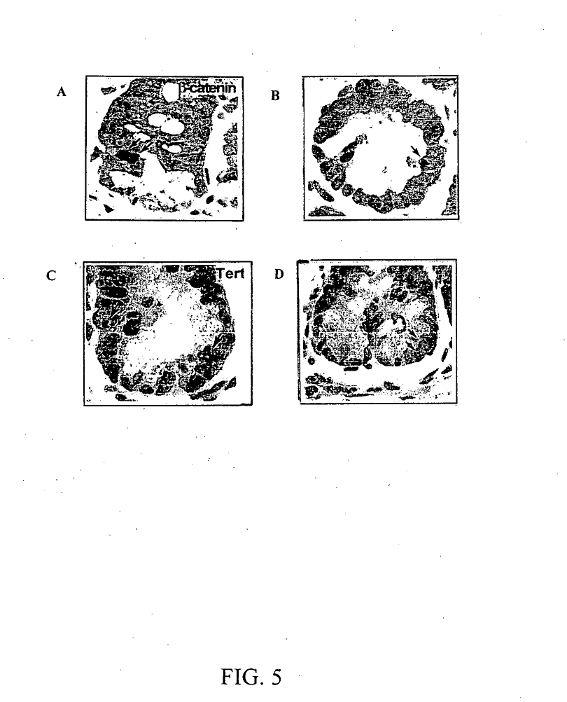

PTEN pathway polypeptides or proteins are those that are encoded by PTEN pathway genes, which include, but are not limited to the following: PTEN, PI3K, AKT, Tert, β-catenin, P27, and BAD genes, and mutant genes derived therefrom. The PTEN pathway, also called the PTEN/PI3K/AKT/Tert/β-catenin pathway, is depicted diagrammatically in FIG. 5B. The PTEN pathway is regulated by Noggin and BMP, which function in a diametrically opposite manner. Noggin binding to Bmpr1a receptor releases BMP inhibition of ISC function, through a cascade of increased levels of activated P-PTEN, P-AKT, β-catenin, and Tert, resulting in ISC proliferation necessary to regenerate dead or lost intestinal epithelial cells in the intestine. In contrast, high BMP activity at the tips of the villi induces increased BAD activity and intestinal cell death; whereas Bmpr1a mutant villi, nonresponsive to BMP signaling, exhibited decreased apoptosis due to loss of BAD signaling.

A regulator is a molecule that regulates an activity of a cell. Regulators include, but are not limited to, BMP, Noggin, or Ly294002. A regulator may cause increase or decrease in an activity of a cell or cell population such as proliferation, self-renewal, differentiation, polyposis, or tumorigenesis. An activator is a regulator that causes an increase in activity. An inhibitor is a regulator that causes a decrease in activity or prevents the occurrence of an activity.

A selectable marker is a marker that is inserted in a nucleic acid sequence that permits the selection and/or identification of a target nucleic acid sequence or gene. A selectable marker associated with the Bmpr1a gene mutation may identify the presence of the Bmpr1a mutation.

Self-renewal occurs when a cell reproduces an exact replicate of itself, such that the replicate is identical to the original stem cell.

Smad proteins are signal transducers that interact with BMP receptors. Smads are evolutionarily conserved proteins identified as mediators of transcriptional activation by members of the TGF-β superfamily of cytokines, including TGF-β, Activins, and BMP. Upon activation these proteins directly translocate to the nucleus where they may activate transcription (Datta et al). Eight Smad proteins have been cloned (Smad 1-7 and Smad 9). Upon phosphorylation by the BMP Type I receptor, Smad1 can interact with either Smad4 or Smad6. The Smad1-Smad6 complex is inactive; however, the Smad1-Smad4 complex triggers the expression of BMP responsive genes. The ratio between Smad4 and Smad6 in the cell can modulate the strength of the signal transduced by BMP. Smad1,5,8 is also referred to as Smad158. Smad-1 is the human homologue of Drosophila Mad (Mad=Mothers against decapentaplegic). Smad-1 has been shown to move into the nucleus in response to the cloning of the BMP-4. An analysis of various tumors demonstrates that mutations in various Smad genes do not, in general, account for the widespread resistance to TGF-β that is found in human tumors. Smad-8 is a protein from Xenopus laevis distantly related to other Smad proteins, and it modulates the activity of BMP-4.

A stem cell is defined as a pluripotent or multipotent cell that has the ability to divide (self-replicate) or differentiate for indefinite periods—often throughout the life of the organism. Under the right conditions, or given optimal regulatory signals, stem cells can differentiate to transform themselves into the many different cell types that make up the organism. Stem cells may be distinguishable from progeny daughter cells by such traits as BrdU retention and physical location/orientation in the villus microenvironment. Multipotential or pluripotential stem cells possess the ability to differentiate into mature cells that have characteristic attributes and specialized functions, such as hair follicle cells, blood cells, heart cells, eye cells, skin cells, or nerve cells.

A stem cell population is a population that possesses at least one stem cell.

Support is defined as establishing viability, growth, proliferation, self-renewal, maturation, differentiation, and combinations thereof, in a cell. In particular, to support an ISC population refers to promoting viability, growth, proliferation, self-renewal, maturation, differentiation, and combinations thereof, in the ISC population. Support of a cell may occur in vivo or in vitro. Support may exclude apoptosis or cell death-related events.

A vector is an autonomously self-replicating nucleic acid molecule that transfers a target nucleic acid sequence into a host cell. The vector's target nucleic acid sequence can be a Wt or mutant gene, or fragment derived therefrom. The vector can include a gene expression cassette, plasmid, episome, or fragment thereof. Gene expression cassettes are nucleic acid sequences with one or more targeted genes that can be injected or otherwise inserted into host cells for expression of the encoded polypeptides. Episomes and plasmids are circular, extrachromosomal nucleic acid molecules, distinct from the host cell genome, which are capable of autonomous replication. The vector may contain a promoter, marker or regulatory sequence that supports transcription and translation of the selected target gene. Viruses are vectors that utilize the host cell machinery for polypeptide expression and viral replication.

Wildtype is the most frequently observed phenotype in a population, or the one arbitrarily designated as “normal.” Often symbolized by “+” or “Wt.” The Wt phenotype is distinguishable from mutant phenotype variations.

### EXAMPLES

### Example 1

An inducible pre-excision Bmpr1a knock-out mouse was generated wherein a Bmpr1a gene could be knocked out in ISC. The mouse was used throughout to study ISC and related signaling pathways. The conditional knock-out Bmpr1a mouse was obtained by crossing a Bmpr1a^{fx/fx} mouse line with an interferon-inducible Mx1-Cre mouse line. Heterozygous Bmpr1a^{+/-} was also used to generate Bmpr1a^{fx/-} as a control.

The Bmpr1a^{fx/fx} mouse line was obtained by targeting vector-mediated insertion of LoxP sites into the Bmpr1a locus of mouse ES cells. To make the vector, one LoxP site was placed in intron 1 of the Bmpr1a gene, and the other two flanking LoxP sites were located in an EcoRI site in intron 2 surrounding a PGK-neo expression cassette. The PGK-neo expression cassette introduced Bg/I and EcoRV restriction sites into the Wt Bmpr1a gene, and the cassette was inserted in reverse orientation relative to the direction of Bmpr1a transcription between the two Bmpr1a intron regions.

The linearized targeting vectors with the expression cassette (PGK-neo) were electroporated into the ES cells that were subsequently cultured in the presence of G418 and FIAU on inactivated STO fibroblasts. Transfected clone 35H3 was characterized by the presence of both a Wt allele (+) and a targeted allele termed the floxP+neo (fn) allele. Subsequent Cre-dependent recombination yielded three alleles: floxP (fx), Δexon 2+neo (Δe2n), and Δexon 2 (Δe2). ES clones containing these alleles were distinguishable on Southern blot analysis with NheI and SacI.

The ES cell clone 35H3 was microinjected into C57BL/6J blastocysts for germ line transmission and implantation into the uterine horns of day 2.5 pseudopregnant foster mothers. Chimeras were identified among progeny mice by the presence of agouti fur, and these progeny were bred with C57BL/6 mice to obtain mutant Bmpr1a^{fx/fx} mice.

Mutant Bmpr1a^{fx/fx} mice were crossed with Mx1-Cre mice (Jackson Laboratory, Bar Harbor, Me., #3556, #2527), yielding litters containing pups with homozygous Mx1-Cre⁺Bmpr1a^{fx/fx} (Bmpr1a mutant), heterozygous Mx1-Cre⁺Bmpr1a^{fx/+}, Wt control Mx1-Cre⁻Bmpr1a^{fx/fx}, and Wt control Mx1-Cre⁻Bmpr1a^{fx/+} genotypes. The resultant Bmpr1a^{fx/fx} mouse line contained a second Exon of the Bmpr1a gene that was flanked by two LoxP sites. This pre-excision Mx1-Cre⁺Bmpr1a^{fx/fx} conditional mutant mouse permitted subsequent recombination activator-induced excision of LoxP-flanked exon 2 of the Bmpr1a gene, resulting in expression of an inactive Bmpr1a receptor polypeptide in the post-excision Bmpr1a mutant mouse.

### Example 2

The pre-excision Mx1-Cre⁺Bmpr1a^{fx/fx} mutant mouse was injected with PolyI:C to induce excision of Exon 2 of the Bmpr1a gene. The Bmpr1a locus was successfully targeted for excision by three injections of the PolyI:C recombination activator at two-day intervals. Thus, it was determined that a post-excision Mx1-Cre⁺Bmpr1a^{fx/fx} mutant mouse possessing inactive and truncated Bmpr1a receptor polypeptides resulted.

Mx1-Cre Bmpr1a mutant pups were injected intraperitoneally with PolyI:C (Sigma-Aldrich, St. Louis, Mo., P-0913, 250 μg/dose) at indicated time points (3 times daily, on alternate days) to induce Cre-mediated LoxP recombination through interferon induction. PolyI:C (250 μg/kg) was injected intraperitoneally on postnatal days 2, 4, and 6 for the early injected group. In addition, pups were injected on postnatal days 21, 23, and 25 for the late injected group. This resulted in mice and, more particularly cells that were Bmpr1a mutants. Specifically, ISCs were Bmpr1a⁻, also known as Bmpr1a knock-outs.

### Example 3

While the Mx1-Cre mouse system alone can be utilized to obtain a viable Bmpr1a knock-out mouse as described in Example 2, a hybrid reporter mouse was made which permitted monitoring of the recombination process. The efficiency of the murine Mx1-Cre line in mediating LoxP-dependent DNA excision in the Bmpr1a gene in intestinal cells was determined by using a hybrid cross between the previously described Bmpr1a Mx1-Cre knock-out mouse and a Z/EG reporter mouse. Clonal inactivation of Bmpr1a in mouse intestines using the Cre-LoxP system was investigated.

The Z/EG reporter mouse was made by introduction of a Z/EG expression vector into R1 ES cells utilizing standard genetic engineering technology. This mouse was designated Z/EG because it expresses both LacZ and enhanced GFPs (EGFP) reporters. The double reporter mouse expressed the LacZ gene that encodes the β-galactosidase enzyme, driven by a ubiquitously active promoter, throughout embryonic and adult stages.

The Z/EG mouse was crossed with the Bmpr1a Mx1-Cre mouse to form Bmpr1a Mx1-Cre Z/EG mice. In the hybrid Mx1-Cre Z/EG reporter mouse, the LacZ indicator gene was flanked with LoxP sites. In addition, the target gene, Bmpr1a was also flanked with LoxP sites. When the LoxP-flanked LacZ gene was deleted by the Cre enzyme in the hybrid mouse, expression of the second reporter, GFP, became activated. GFP indicates successful removal of the first reporter gene, LacZ, mediated by the flanked LoxP. As such, this also indicates removal or mutation of the Bmpr1a gene. As in Example 2, Cre recombinase activity and LacZ excision was triggered by postnatal injection of the recombination activator, PolyI:C. Thus, the presence of LacZ gene expression in cells, as indicated by X-gal staining, indicated the pre-DNA-excision state. In contrast, GFP expression represented the post-DNA-excision state, where both the Bmpr1a and LacZ genes were excised.

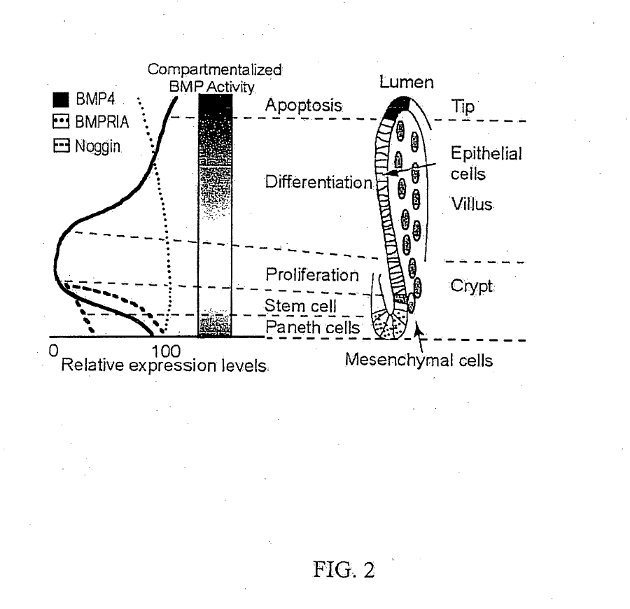

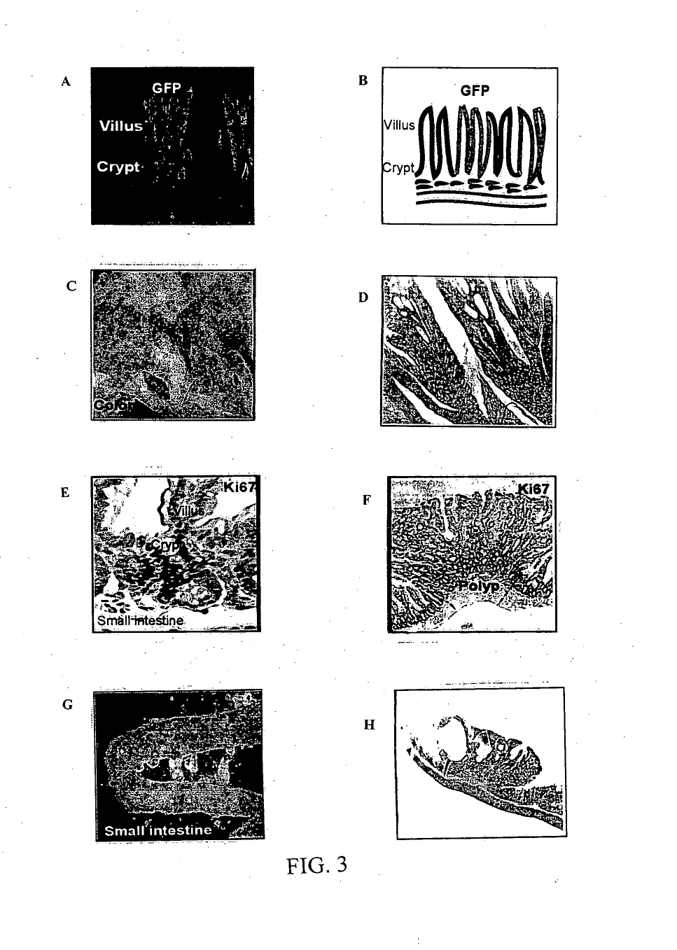

The Mx1-Cre-dependent DNA recombination efficiency analysis in the intestine of the Wt and Bmpr1a mutant Z/EG reporter mice is shown in FIG. 3A, with a diagrammatic illustration shown in FIG. 2. The hybrid mutant mice were injected with PolyI:C to induce excision of Exon 2 of the Bmpr1a gene through recombination. The GFP signal (green) of FIG. 3A indicates successful gene targeting, while the LacZ signal (blue) represents un-targeted cells. PolyI:C induced genetic recombination in the LoxP-flanked Bmpr1a gene and the LoxP-flanked LacZ gene of the hybrid Mx1-Cre Z/EG reporter mouse. Recombination was detected by loss of LacZ expression and gain of GFP expression in the double reporter mouse.

It was determined that deletion of the Bmpr1a receptor gene was clonal because the entire villus/crypt unit was either GFP positive or GFP negative, as depicted in FIG. 3A. This result can be explained by the fact that receptor deletion occurred in an ISC, which then proliferated and differentiated to generate the derived GFP positive cells along the entire villus base to the mid-region and tip axis. GFP negative regions indicated the presence of the Wt Bmpr1a receptor gene. This result suggested that after PolyI:C induced gene deletion occurred in the stem cells, GFP expression occurred in the ISC as well as all lineages differentiated therefrom and present throughout the entire crypt/villus unit. Conversely, the cells emanating from ISCs that were not targeted retained LacZ expression, but not GFP expression, indicating the presence of Wt Bmpr1a throughout the population. The results also indicated that when a mutation occurred, the entire villus crypt region was impacted.

It was determined that polyps and tumors were clonally expressed. Correspondingly, when the Bmpr1a receptor was functionally ablated in Bmpr1a mutant mice, polyps and tumors appeared in a clonal manner. The pathological appearance of polyps in mutant mice resembled the phenotype observed in human JPS.

### Example 4

LacZ gene expression of the β-galactosidase enzyme was detected by substrate staining with X-gal, a 5-bromo-4-chloro-3-indolyl-β-D-galactopyranoside substrate (Sigma-Aldrich, St. Louis, Mo., FW=408.6, B4252), chemically characterized as an indole derivative. In X-gal staining, formalin-fixed intestine was exposed to X-gal solution. After PBS wash, sections were counterstained with Nuclear-Fast-Red (Sigma-Aldrich, St. Louis, Mo., N-020).

For GFP staining (Clonetech, Palo Alto, Calif.), intestinal tissue was fixed in zinc formalin overnight, PBS washed, and immersed in 30% sucrose in PBS at room temperature overnight. On the second day, the tissue was embedded in an OCT solution (ornithine carbamyltransferase, Miles Diagnostics, Inc., Elkhart, Ind.) and snap-frozen, then sliced into 8 μm thickness sections, mounted with DAPI blue fluorescent counter stain, and prepared for imaging. DAPI preferentially stains double-stranded DNA, attaching to adenine-thymine (AT) clusters in the DNA minor groove. DAPI stains nuclei, with little or no cytoplasmic staining. After DAPI counterstaining, slides were then ready for imaging.

### Example 5

Because it was shown that the Bmpr1a mutant was clonal, it was hypothesized that this would have an impact on BMP signaling throughout the crypt/villus, as well as other signals. As will be shown, differentially localized BMP activity defines the formation of discrete zones in the villi in which ISCs undergo a sequential developmental process. The zones are defined or illustrated by the presence of various proteins in varying amounts. The affected signals or proteins include BMP, Noggin, P-Smad1,5,8, and Bmpr1a. Before impact of the mutant could be examined it was necessary to understand and illustrate the distribution of these signals in a Wt system. To investigate the potential roles of the BMP signal in regulating ISC development, it was first determined that the expression patterns of BMP4, its antagonist Noggin, and the receptors, Bmpr1a and Bmpr1b, should be examined and then compared to mutant mice 22 days after poly ISC treatment.

ISCs in Wt mice were identified by a BrdU-retaining assay performed with an eosin counterstain. Brd-U specifically stains proliferating ISCs. The ISCs were identified as being located at the fourth or fifth cell position from the base of each crypt and superior to paneth cells (located in the crypt bottom with multiple granules in the cytoplasm) in the small intestine, as shown in FIGS. 1A and 1B. In FIG. 1A, the arrow (V) indicates the position of the ISCs under moderate magnification. In FIG. 1B the tissue was co-stained with Brd-U and lysozyme antibody. The lysozyme antibody stained granules located in the paneth cells. As can be seen in FIG. 1B, the paneth cells were located below the ISC. The position of the ISC in the villus is schematically illustrated in FIG. 1C. Thus, the location of the ISC in the crypt/villi region was identified.

BMP4 LacZ mice were used to identify the location of BMP in intestinal tissue. Expression of LacZ reflects the level and distribution of endogenous BMP4 mRNA, and it was found that BMP4 mRNA was expressed in mesenchymal cells and adjoining spaces. It was observed that LacZ, (which indicated BMP4 expression) was expressed in mesenchymal cells from the basement membrane, extending to the space along and beneath the epithelial cells of each villus (FIG. 1D). The tissue was next co-stained with BMP4 and Brd-U. BMP4 was used to stain mesenchymal cells and Brd-U ISCs. BMP4 was detected in the mesenchymal, but not ISCs. The presence of BMP4 mRNA extended to the space along and beneath the epithelial cells of each villus and in the mesenchymal cells adjacent to the ISCs, as shown in FIG. 1D. Higher magnification observed in FIG. 1E, revealed mesenchymal cells adjacent to ISCs, suggesting that mesenchymal cells that expressed BMP4 could influence ISC growth, self-renewal, and proliferation. Thus, the BMP4 was expressed in the mesenchymal cells adjacent to the region where the ISCs were located, as shown in FIGS. 1D and 1E. Relative expression of BMP4 is illustrated in FIG. 1F. BMP4 is present in the Wt throughout the crypt and villus.

Noggin is a BMP antagonist and competes with BMP for binding to the Bmpr1a receptor. Tissue samples from BMP LacZ mice were stained with LacZ and counterstained with eosin to locate the presence of Noggin. As shown in FIGS. 1G and 1H, most Noggin was located in the basement membrane cells, adjacent to the bottom of the crypt and in some ISCs, reflecting a periodic event. Noggin production fluctuated and was not detected in other sections, and there was dynamic change in Noggin levels expressed among ISCs. The Noggin production in ISCs and in basement membrane is shown in the diagram in FIG. 11. Thus, Noggin was observed in a particular region, the basement cells, of the intestinal cells.

The distribution of Bmpr1a receptor protein (Bmpr1a) in intestinal tissue was investigated. HEC (red) conjugated secondary antibody was used for recognition of anti-Bmpr1a serum and counterstained with hemoxylin (blue). Bmpr1a was detected in most of the epithelial cells in the villi and crypts using immunohistochemical staining, as shown in FIG. 1J. The level of Bmpr1a expression varied in different regions along the crypt/villus axis. Bmpr1a was lowest or non-detectable in the upper part of the crypt, due to non-expression of Bmpr1a. This zone was identified as the proliferation zone, as depicted diagrammatically in FIGS. 1L and 2. Bmpr1a receptor was present at its highest levels both at the tip of the villus and at the bottom of the crypt. Bmpr1a immunostaining is not compatible with BrdU staining procedures. To overcome this, Bmpr1a was co-stained with 14-3-3ζ. The Bmpr1a receptor is highly expressed in ISCs, as shown by its co-staining with an ISC marker 14-3-3ζ. Thus, the diffusible BMP signal generated by the mesenchymal cells is able to influence epithelial cells (including the ISCs) for self-renewal, differentiation, and apoptosis through the receptor Bmpr1a.

Tissue was stained to show the distribution of P-Smad1,5,8, which reflects BMP activity. The distribution pattern of the BMP downstream component, P-Smad1,5,8, confirmed that the level of BMP activity varied from zone to zone, as shown in FIG. 1M. In the lowest portion of the villi, P-Smad1,5,8 appeared in reduced levels, with Smad activity increasing towards the tips of the villi, as shown in FIG. 1M. P-Smad1,5,8 was co-stained with Brd-U. P-Smad1,5,8 in the ISC, relative to paneth cell and crypt regions, is shown in FIG. 1N.

A summary illustration graph depicting relative BMP, Bmpr1a, and Noggin activity expression levels is presented in FIG. 2. In the crypt region, dual regulation by Noggin and Bmpr1a led to lower BMP activity in the bottom of crypt, as shown in FIG. 2. BMP activity was higher at the tip of the lumen, where intestinal cells underwent apoptosis. In the stem cell zone, the BMP activity was high, as shown in FIG. 2; however, BMP activity at the base of the villus varied inversely relative to the level of Noggin expression, as shown in FIG. 2, where increased Noggin led to decreased BMP activity. The lowest BMP activity occurred in the region of the upper-crypt, due to the absence of expression of Bmpr1a on the transient amplifying (TA) cell progenitors in the proliferation zone, as shown in FIGS. 1J and 1K and illustrated diagrammatically in FIG. 2. This gradient distribution of Bmpr1a was more pronounced in the BMP-transgenic intestine, in which over-expression of BMP4 was driven by a 2.4 kb BMP4 promoter, as shown in FIG. 1D. BMP4 activity appeared at a relatively uniform level along the axis of the crypt/villi. BMP activity was lowest in the upper crypt region, but higher in the ISCs, as shown in the black and white shaded graph at the right of FIG. 2. The BMP activity in ISCs fluctuated with the presence of Noggin in those cells. Localized BMP activity was highest at the villi tips, but ranged from low to intermediate activity in the mid-regions spanning the crypt region to the tips.

A change in the level of Noggin expression in the crypt bottom and in the ISCs, as illustrated in FIG. 2, functioned to control ISC properties through regulation of BMP activity. This finding is consistent with prior reports that Noggin was shown to antagonize the BMP signal, and to regulate the stem cell niche during neurogenesis. In the crypt region, paneth cells exhibited low BMP activity which was, in turn, reciprocally dependent upon the Noggin activity level. If Noggin was high, BMP was low, and vice versa. Noggin was expressed at high levels at the villus bottom, but Noggin dropped dramatically outside this localized region.

Bmpr1a receptor activity was present at high levels at the bases and tips of the villi. It is noteworthy that paneth cells and ISCs were located at the villus bottom, where Bmpr1a receptor was highly expressed. However, Bmpr1a exhibited low to intermediate level activity in the mid-regions of the villi.

It is concluded that the interplay between Noggin activity, as a BMP antagonist, in the intestinal region in combination with the Bmpr1a receptor density on individual intestinal cells enables the precisely BMP-tuned regulation of the responding epithelial cells, particularly stem cells. Thus, the “BMP activity readout” (“BMP activity”) varies along the crypt/villus axis as a result of the combination of the levels of expression of these three components, signal, receptor, and antagonist, as shown in FIG. 2. The localized BMP activity exhibited along the villus corresponds to the zonal map of self-renewal, proliferation, differentiation, and apoptosis, where ISCs undergo a sequential development process, as shown in FIG. 2. This is also illustrated in FIG. 17. Transient expression of Noggin in the intestinal niche can function to control ISC properties through regulation of the BMP signal.

### Example 6

Polyposis induced Mx1-Cre⁺Bmpr1a^{fx/fx} mutant mouse pups were investigated as a potential animal model for human JPS. As mentioned, the pups were injected with PolyI:C on postnatal days 2, 4, and 6 for the early injected group. The later injected group was given PolyI:C on postnatal days, 21, 23, and 25. Both of these two PolyI:C induced groups (early and later injected) caused formation of mutant, inactive Bmpr1a genes and receptors in ISCs. The Bmpr1a mutant mice, induced at either injection time window, started to develop multiple polyps or polyposis in the small intestine (in mice with later injection of PolyI:C after 4-6 months), or large intestine region (in mice with earlier injection of PolyI:C after 2 months), as shown in FIGS. 3C and 3D and FIGS. 3G and 3H, respectively. It should be noted that when a mutant is referred to herein these representative results were obtained from Bmpr1a mutant mice injected at either early or late time windows.

Polyps were observed 2 months post injection in the colon of the entire earlier injection group, FIGS. 3C and 3D, and in the small intestines 5 months post injection, from the jejunum to the ileum (between 15-25 cm, measuring from the stomach), FIGS. 3G and 3H. Bmpr1a mutant mice exhibited similar features characteristic of human JPS, with focal hamartomatous malformations and slightly lobulated lesions with stalks. Histological analyses revealed that the murine polyps enclosed abundant cystically dilated glands with normal epithelium, but showing hypertrophic lamina propria and mucosal cysts. Mice also started to show general signs of histopathology manifested as anemia with paled paws. Importantly, results from the Bmpr1a mutants illustrated that when a mutation affects BMP signaling, Bmpr1a receptor inactivation can cause polyposis.

Increasing the number of ISCs potentially produces multiple crypts through a postulated mechanism of crypt fission triggered by symmetrical stem cell division, as illustrated diagrammatically in FIG. 11C. Increasing ISCs relate to polyp formation. The crypt fission mechanism is supported by three findings: (1) the significant increase in the number of crypts in the tumor region of the Bmpr1a mutant mice; (2) the fact that duplex stem cells, which are positive for P-PTEN or AKT-S473, were found in the same crypt, and (3) the presence of symmetric stem cell division patterns in the tumor region. A diagram of tumor formation in Bmpr1a mutant mice, showing crypt fission due to symmetrical division of ISCs is illustrated diagrammatically in FIG. 11D.

It was observed in the proliferation zone of mutant mice that non-expression of Bmpr1a resulted in the lack of BMP-mediated suppressive activity, resulting in intestinal stem cell proliferation. Inactivation of Bmpr1a receptor in the intestinal cells of the Mx1-Cre-Lox mutant mouse pups led to the formation of profuse polyps throughout the gastrointestinal tract, resembled human juvenile polyposis. An increase in proliferating progenitor cells were present in the region enriched with multiple crypts, as will be discussed.

### Example 7

As discussed in Example 6, when BMP is blocked, the result is abnormal gastrointestinal development. Expansion in the proliferation zone results from blocking BMP signaling. This block in the BMP signal, leads to severe gastrointestinal dysplasia. Blocking BMP affects ISC developmental processes: self-renewal, proliferation, differentiation, apoptosis, or some combination. Proteins or polypeptides that interact with BMP will resultingly decrease or increase. Changes in the amount of the protein provide information on the fate of cells in the intestine and the mechanisms that control cell fate. Wt (normal) and mutant intestinal cells in mice were analyzed to see changes in various polypeptides. The mutants were the Bmpr1a knock-outs of Examples 1 or 3. Tissue samples were taken, fixed, and stained for Ki67, P-Smad1,5,8, p27^{kip}, P-PTEN, P-AKT, β-catenin, and Tert.

Ki67 is a marker for proliferating cells, but not ISC. The presence of chromosomal proliferation-associated marker Ki67 was examined in normal and Bmpr1a mutant villi to determine the effects of BMP activity on cell proliferation. In the Wt intestinal tissue, the Ki67 marker stained cells in the crypt region, apart from the bottom of the crypt, which corresponded with the absence of expression for the BMP. Ki67 exhibited the brown coloration as shown in FIG. 3E. The observations suggested that the BMP signal, acting through the Bmpr1a receptor, defined the contours of the proliferation zone by inhibiting cell proliferation outside the zone. Further proof for this view was obtained by examination of the Ki67 marker staining of intestinal tumor cells of Bmpr1a mutant mice, as shown in FIG. 3F. The tumor cells had significantly more Ki67 compared to normal cells. The Ki67 staining distribution in mutant tumor cells revealed a dramatic 5 to 10 fold increase in Ki67 over Wt cells, with corresponding increases in cell number. The mutant results in a significant cell population increase in the proliferation zone.

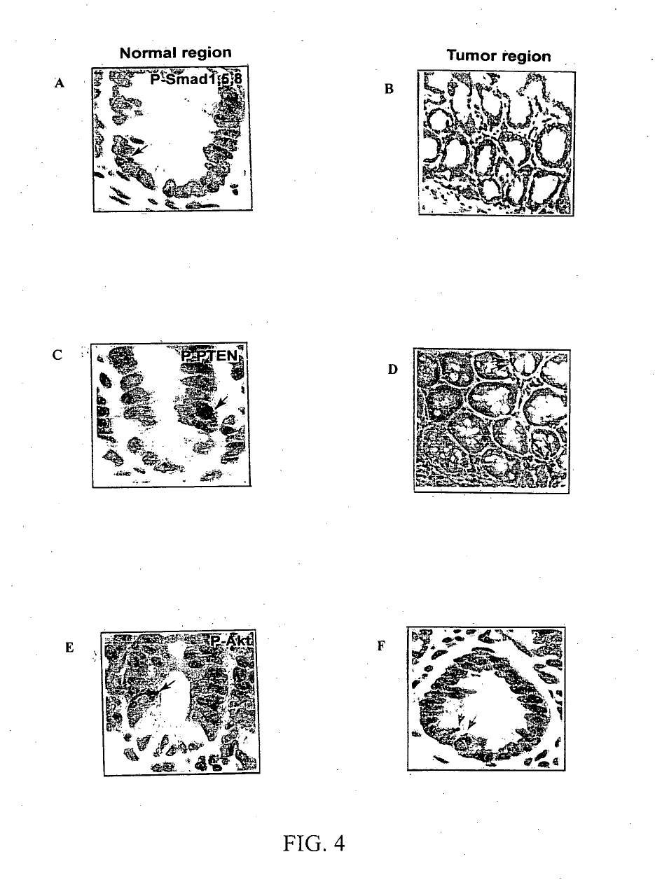

P-Smad reflects the activity of BMP. When BMP activity is reduced or eliminated, P-Smad activity is correspondingly reduced. In the normal tissue, depicted in FIG. 4A, P-Smad1,5,8 was present in the ISC, and also in the crypt/villus, similar to BMP. However, in tumorous tissue of Bmpr1a mutant mice, as shown in FIG. 4B, P-Smad1,5,8 signals were strikingly absent. Taken together, these results support the concept that inactivation of the Bmpr1a receptor in mutant mice resulted in blocking of the BMP signal to proliferating cells, with concomitant down-regulation of P-Smad1,5,8.

In the ISC self-renewal zone, the BMP signal, produced by mesenchymal cells, apparently controls self-renewal through the regulation of PTEN (Phosphotase and Tension homolog) activity and restricted activation of ISCs by stimulating p27^{Kip}. The BMP signal likely increases the PTEN protein level through inhibition of ubiquitin-dependent PTEN degradation

To determine Bmpr1a gene mutation effects on ISCs, in general, and on ISC self-renewal in particular, the presence of the inactivated phosphorylated form of PTEN (P-PTEN:PTENS380, T382, S383) in intestines was examined. The P-PTEN signal was specifically detected in ISCs where self-renewal occurs, as shown in FIG. 4C. As can be seen in the Wt, P-PTEN was present in a defined self-renewal zone associated with ISC. The presence of P-PTEN was also observed in mutant tumor tissue, as shown in FIG. 4D indicating that as the ISCs proliferated in the mutant, the P-PTEN was present. In particular, increased self-renewal in the mutant resulted in an increase in P-PTEN. As the ISCs increased numerically 5-6 fold in tumors, and the crypt numbers increased, the amount of P-PTEN increased. These findings support the inhibitory role of the BMP receptor in suppressing ISC self-renewal and proliferation, putatively via P-PTEN expression.

PTEN is a PI3K inhibitor, and AKT is the main signal occurring downstream of the PI3K pathway. Therefore, it was reasonable to examine whether AKT was activated when P-PTEN was present in ISCs. As predicted, the activated form of AKT (AKT-S473 or P-AKT) was associated with the ISCs in the self-renewal zone, as shown in FIG. 4E. The P-AKT was present in the tumor cell in a greater amount, as shown at FIG. 4F. The tumor had increased self-renewal. Thus, it was determined that activated P-AKT was specifically expressed in ISCs, where Pr3 kinase and activated P-AKT both regulate self-renewal properties of ISCs. This observation led to the hypothesis that AKT may play a role in regulation of self-renewal of ISCs. Taken together, results observed in tumor and nontumor tissue of Bmpr1a mutants, shown in FIG. 4A-4F, clearly indicated that the BMP signal, derived from mesenchymal niche cells, played a critical role in inhibiting self-renewal of ISCs through homeostatic stimulation of PTEN.

β-catenin plays a role in regulating stem cell self-renewal and can be activated by AKT through GSK3β. Unexpectedly, β-catenin was found to be nuclear-localized in mitotic ISCs, or self-renewing ISC, as shown in FIG. 5A. In contrast, in non-mitotic ISCs, β-catenin was asymmetrically localized to the membrane adjacent to the mesenchymal cells, as shown in FIG. 5B. The nuclear-localization of β-catenin in mitotic ISCs and cytoplasmic localization in non-mitotic ISCs indicates that expression of β-catenin in the nucleus is associated with ISC proliferation and self-renewal. β-catenin expression, revealed by DAB (brown) staining, was shown to be localized in the intestinal stem cell (top cell) and also in the potential mesenchymal niche cell (bottom cell) located outside of the crypt. The mesenchymal niche cell may be a myofibroblast cell type.

The expression pattern of Tert, encoding the catalytic subunit of Telomerase, was examined. Consistent with reports that Tert is required for self-renewal of stem cells, in general, specific expression of Tert was detected in ISCs, as shown in FIG. 5C. Tert was also expressed in the mutant cells, as shown in FIG. 5D. Tert's presence in ISCs is also in agreement with a report that AKT can enhance Telomerase activity through specific phosphorylation of its catalytic subunit, and with a previous observation that Tert was specifically activated in ISCs. These results suggested that the BMP signal can operatively inhibit ISC self-renewal via activation of the PTEN-PI3K-AKT-Telomerase (Tert) cascade. Tumor regions derived from the Bmpr1a mutant mice were examined, and it was found that in BMP's absence, P-PTEN, Tert, P-AKT, and β-catenin increased. In addition to P-PTEN and Tert markers, AKTS473 and β-catenin markers were detected specifically in ISCs in the crypts of the tumor region.

To confirm the specificity of the detection of P-PTEN and AKT-S473 in ISCs, Ki67 co-staining of these signals was performed. This staining procedure revealed that first, the cells that were positive for either P-PTEN or P-AKT were not in a highly cycling state (Ki67-negative). Secondly, the locations of the P-PTEN or AKT-S473 positive cells were at the base of colon, where ISCs were presumably to be located, as shown in FIGS. 2N and 20. These results supported the view that these P-PTEN or AKT-S473 positive cells were in fact ISCs and not proliferating cells.

Telomerase is required for stem cell self-renewal, and AKT was demonstrated to enhance this activity through specific phosphorylation of its catalytic subunit. Thus, AKT may potentially be involved in the regulation of ISC self-renewal through the activation of both β-catenin and Telomerase during ISC division.

These foregoing observations support the conclusion that the BMP signal functions to arrest ISC growth, and to withdraw progenitor cells from their cell cycle when the cells migrate into the differentiation zone. They further show that when BMP is blocked, increased cell proliferation occurs.

In conclusion, activation of AKT is required for stem cell self-renewal by maintaining their proliferation potential through the activation of β-catenin and Telomerase. In addition, results demonstrated that BMPs trigger an alternative pathway that acts in ISCs to regulate ISC self-renewal. Taken together, these results show that in this BMP signal pathway, activation of PI3K-AKT-β-catenin-Telomerase, as a consequence of the loss of PTEN-function, led to an expansion in the stem cell population. Thus, the PI3K-AKT pathway appears situated centrally at the hub of these various signal pathways as a common component of these regulatory systems for stem cell self-renewal and maintenance. Also identified were markers for self-renewal identification

### Example 8

Next it was examined whether ISC self-renewal is affected in the Bmpr1a mutant mice, and if so, which molecules and underlying signal pathways are involved. Mutations in either the Bmpr1a or phosphatase and tensin homolog (PTEN) genes give rise to syndromes with a different spectrum of symptoms but which include gastrointestinal polyps. Since the BMP signal can stabilize PTEN, potentially through inhibition of phosphorylation, this raised the possibility that the BMP signal positively regulates PTEN activity.

To test this hypothesis, the inactivated (phosphorylated) form of PTEN (P-PTEN:PTENS380, T382, S383) in intestine sections was examined. Strikingly, it was determined that the P-PTEN signal in cells located at the ISC position retain the labeled BrdU (FIGS. 15A and 15B) and are also negative for Ki67 (FIG. 15C), indicating that the cells are not TA progenitors. In addition, it was believed that if P-PTEN can specifically recognize ISCs in the intestine, the ISCs should also be recognized in the colon. Indeed, immunohistochemical staining confirmed this; P-PTEN positive cells are located at the bottom of colon crypt, the reported ISC position in this region (FIG. 15C). Therefore, P-PTEN specifically recognizes ISCs and is used as an ISC specific marker hereafter.

As PTEN is an inhibitor of PI3K, and AKT is the main downstream component of the PI3K pathway, it was analyzed whether AKT is activated when PTEN is in the form of P-PTEN in ISCs. The activated form of AKT (AKT-S473 or P-AKT) was detected and predominantly existed in the BrdU-retaining cells (FIGS. 15E-15F), marking the ISCs. Furthermore, like P-PTEN-positive cells, P-AKT-positive cells were negative for Ki67 and located at the crypt base in colon sections (FIG. 15G). Thus, both P-PTEN and P-AKT associated with ISCs specifically. Since AKT targets many downstream molecules, including β-catenin through GSK3β, telomerase, and BAD, expression patterns of these molecules were examined and commonly expressed in self-renewing cells.

B-catenin plays a role in regulating stem cell self-renewal. Although β-catenin is known to be a key downstream factor in responding to Wnt signaling, it is also reported to be activated by AKT through GSK3β. It was observed that β-catenin is in the membrane-associated form in ISCs evidenced by its association with BrdU-R (FIG. 151). The nuclear-accumulation of β-catenin was associated with inactivated PTEN (P-PTEN) in ISCs (FIG. 15J) and is also seen in dividing ISCs (FIG. 15K). These observations lead us to the hypothesis that is consistent with a previous report that inactivation of PTEN is responsible for the nuclear-accumulation of B-catenin through activation of AKT and subsequent suppression of GSK3B. Thus, nuclear-accumulation of B-catenin may be required to activate the arrested ISCs by stimulating their division (FIG. 15K). Further, it was observed that no nuclear-accumulation of β-catenin was seen in the proliferation zone, and this may be due to loss of activated AKT.

Telomerase is also required for stem cell self-renewal and AKT can enhance this activity through specific phosphorylation of its catalytic subunit (Tert). Specific expression of Tert was detected in ISCs (FIGS. 16E-16S). But how Tert expression is regulated by AKT is not yet clear. As AKT is reported to be able to activate c-Myc through GSK3β, and c-Myc is able to transactivate Tert through its binding to an E-box site in the Tert ptomoter, Tert may be transcriptionally regulated by c-Myc and/or post-translationally activated by AKT phosphorylation. Thus, AKT may be involved in the regulation of ISC self-renewal through activation of both B-catenin and telomerase during ISC division.

It was concluded that the BMP signal plays a role in inhibiting ISC self-renewal partially via a cascade of PTEN-PI3K-AKT-β-catenin/Telomerase. If this hypothesis is correct, an increase in the self-renewal capacity of ISCs should occur when the BMP signal is blocked. To address this, the stem cell compartment was examined, which is within the multiple crypts, in the polyp regions derived from the Bmpr1a mutant mice and found that the overall number of ISCs as characterized by the various parameters outlined above, was significantly increased, and multiple doublet ISCs were also seen (FIGS. 15D, 15H, 15I, 16R). In the crypts of the polyp region, P-PTEN, AKTS473, β-catenin, 14-3-3ζ, and Tert were detected specifically in these ISCs (FIGS. 15D, 15H, 15I, 16C, 16R).

### Example 9

Western blots were performed on PTEN pathway numbers. For Western blot analysis, intestinal tissue was homogenized in a cocktail of 1 ml lysis buffer (100 mM Tris-Hcl, pH 6.8, 2% SDS, and proteinase inhibitor supplied by Roche. The supernatant was collected after centrifugation. Protein extracts (75 μg/well) were fractionated on SDS-PAGE gel and transferred onto nitrocellulose membrane. The membrane was blocked using casein blocker (Pierce), and was incubated with appropriate primary and secondary antibodies (1:5,000 dilutions) in casein blocker. The membrane was developed after washing with TBS-T solution (TBS plus 0.05% Tween-20) and immersing in chemiluminescent reagents.

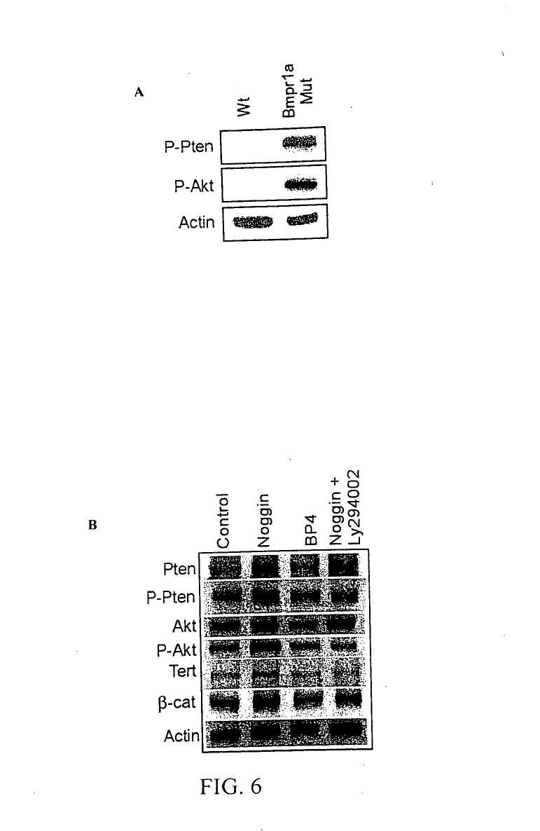

Data from Western blot hybridization experiments indicated that the level of P-PTEN and P-AKT was significantly increased in the Bmpr1a mutant intestine over Wt levels, as shown in FIG. 6A. When the BMP signal was blocked in the Bmpr1a mutant mouse, P-PTEN was significantly increased 5-6 fold over Wt, as shown in the electrophoretic gel of FIG. 6A, where actin was used as a positive control Western blot. The results supported the view that the BMP signal in ISCs regulated PTEN activity, which correspondingly suppressed the Pr3 kinase pathway. When the BMP signal was ablated in the Bmpr1a mutant, PTEN was inactivated, which led ultimately to ISC self-renewal.

### Example 10

Wt mice were co-stained with Bmpr1a and Ki67, P-Smad1,5,8, and Ki67, and p27^{Kip}. The intent was to compare proliferating cells (Ki67⁺) with markers which reflect BMP activity.

Ki67 and Bmpr1a co-staining in a Wt mouse is shown in FIGS. 12A and 12B, where Ki67 (red) is a marker for proliferation, and Bmpr1a staining (green) detects the Bmpr1a receptor. In the Wt ISCs, Ki67 was negative in the villus, indicating that the ISCs were in either resting or slow dividing states, rather than in a highly proliferating state. The proliferation zone, containing Ki67-positive stem cells, is depicted in FIG. 12B. Bmpr1a receptor was not expressed in the proliferation zone, as shown in FIG. 12B. FIG. 12A shows green Bmpr1a staining throughout the entire length of the villi, with Ki67 staining (red) appearing in both the crypt/villus regions. Ki67 staining was most pronounced in the upper crypt region, whereas Ki67 was negative in the Bmpr1a staining paneth cells, as shown in FIG. 12B. The Ki67⁻ stem cell depicted was not undergoing a cell division cycle and was located below the crypt region, as shown in FIG. 12B.

Co-staining of P-Smad1,5,8 (green) and Ki67 (red) in Wt murine intestinal cells is shown in FIGS. 12C and 12D, where the anti-P-Smad antibody utilized is directed against the inactivated, phosphorylated form of the molecule, P-Smad1,5,8. P-Smad1,5,8 activity occurs downstream from BMP's initial interaction with Bmpr1a receptor. P-Smad1,5,8 staining was prevalent along the entire length of the villus, as shown in FIG. 12C. Ki67 staining shows the red proliferation zone located at the base of the villus, as shown in FIG. 12C. In the crypt region, shown in FIG. 12D, the P-Smad1,5,8 activity distribution in intestinal stem cells correlated with the presence of BMP activity in the ISCs. P-Smad1,5,8 appeared in high concentration in non-proliferating Ki67⁻ intestinal cells in the differentiation region located above the crypt region (proliferation zone). Taken together, results indicated that intestinal cells expressing P-Smad1,5,8 are in an arrested cell state, with the non-dividing stage situated along a differentiation pathway. In addition, ISCs appeared to cycle slowly, as evidenced by the pattern of weak to no staining of Ki67, as shown in FIGS. 12A-12D.

The P27^{Kip} distribution pattern was similar to the P-Smad1,5,8 distribution pattern observed in FIGS. 12C and 12D. Horseradish peroxidase-conjugated anti-p27^{Kip} antibody was used with diaminobenzidine (DAB) substrate (brown), against a hematoxylin counterstain (blue). A p27 gradient was observed in FIG. 12E. p27 villus staining is provided in FIGS. 12E and 12F. High levels of p27^{Kip} were found in the ISCs, with low levels detected in the proliferation zone. Low to intermediate levels were found in the villi, with highest levels found at the tips of the villi, as shown in FIGS. 12E and 12F. It was concluded that when cells were proliferating, Ki67+, the markers associated with BMP, were reduced or not present. As such, cells proliferate when BMP binding is inhibited.

### Example 11

Related to the foregoing Example, proliferation marker analysis was conducted in mouse Wt intestinal tissue, and compared to Bmpr1a mutant tissue.

Inactivation of Bmpr1a receptor in mutant mice resulted in substantially increased proliferation of cells in the proliferation zone, as detected by Ki67 staining. Mutant tumors exhibiting a 5-10 fold increase in proliferation over the Wt was observed. Moreover, in the mutants, P-Smad1,5,8 expression was down-regulated, along with the deletion of Bmpr1a. p27 was also down-regulated and expressed in the cytoplasm, indicating that Bmpr1a did not control the cell cycle. Polyps and tumors were clonally expressed in Bmpr1a mutant mice, indicating the mutant mouse might be used as a model organism for study of the pathogenesis and treatment of human JPS.

The cells were first stained with Ki67, a proliferation marker, and DAPI counterstain, as shown in FIGS. 13A and 13B. Tumor cells were intensely stained with Ki67, as shown in FIG. 13B. This staining pattern contrasted with the more regular staining pattern observed in the Wt intestinal tissue, as shown in FIG. 13A. Crypt proliferating cell numbers dramatically increased in the mutant tissue compared to Wt tissue, as shown in FIG. 13B. DAPI revealed nuclear staining throughout the crypt area, as shown in FIG. 13A. A representative Ki67 negative putative stem cell in Wt tissue is depicted at the yellow arrow in FIG. 13A.

In FIGS. 13C and 13D, the colon of Wt and mutant mice were co-stained with P-PTEN and Ki67 markers. The P-PTEN staining ISC, at the white arrow, as shown in FIG. 13C, was located at the bottom of crypt region in a Ki67 negative ISC. In the small intestine, P-PTEN staining ISCs appear in the 4th or 5th cell position from the base of the villus. These results confirmed P-PTEN specificity occurred in arrested or slow dividing ISCs. In colon tumors, duplicated cells stained with P-PTEN, as shown at the two white arrows in FIG. 13D, illustrated that symmetric division occurred among self-renewing ISCs situated at the bottom of the crypt.

Thus, proliferation marker analysis with Ki67 showed that Bmpr1a mutant mice exhibited dramatically increased numbers of proliferating cells in comparison with Wt. Co-staining of Ki67 with P-PTEN antibodies revealed that predominantly Ki67 negative, nondividing ISCs were stained positive for P-PTEN in Wt intestinal tissue. However, in colon tumors of Bmpr1a mutant mice, P-PTEN staining of ISCs was also observed, along with symmetric division along the crypt.

### Example 12

Noggin and BMP treatment of in vitro cultivated intestinal organ tissue demonstrated that the addition of the competitive inhibitor Noggin to Bmpr1a receptor-bearing ISCs caused activation of P-PTEN and P-AKT, β-catenin and Tert along the ISC pathway. In particular, it was demonstrated that Noggin released BMP-mediated inhibition, as shown schematically in FIG. 11B. This Noggin-induced activation caused ISC self-renewal and proliferation.

To functionally prove that BMP regulates β-catenin and Tert through PTEN and AKT, segments of small intestines in organ cultures were cultivated in vitro. Noggin and BMP proteins were placed in Affigel beads, as described hereinafter and positioned in operative contact with intestinal tissue in vitro. These segments were maintained in medium containing either 25 ng/ml BMP4 or Noggin, or Noggin plus Ly294002, an inhibitor of the PI3K pathway. To ensure exposure of intestine segments to sufficient concentrations of Noggin or BMP4, BMP4-soaked or Noggin-soaked beads were injected directly into the interior of the corresponding segments, as illustrated in the photograph of FIG. 13E. Organ culture was carried out in the following medium: 50% of DMEM-1 without calcium, 40% supplemented F-12/Mixture (Biosource), 10% FBS, 1% Pen-Strep, and 1% Fungizone. Additional alternative reagents were added in the following concentrations: 2 mM/ml of Ly294002 (Sigma), 25 ng/ml BMP4, or 25 ng/ml of Noggin (R7D system). Affigel blue beads (100-200 mesh, BioRad) were soaked in 500 mg/ml of Noggin, in 500 mg/ml of Noggin, or 500 mg/ml of BMP4 at RT for one hour, and were then injected into 0.5 inch intestinal segments (10 beads/segment). After culturing for four (4) hours, during which time, peristaltic movement continued in the intestinal segments, these segments were harvested and subjected to analyses.

For Western blot analysis, intestinal tissue was homogenized in a cocktail of 1 ml lysis butter (100 mM Tris-Hcl, pH 6.8, 2% SDS), and proteinase inhibitor (supplied by Roche). The supernatant was collected after centrifugation. Protein extracts (75 μg/well) were fractionated on SDS-PAGE gel and transferred onto nitrocellulose membrane. The membrane was blocked using casein blocker (Pierce), and was incubated with appropriate primary and secondary antibodies (1:5,000 dilutions) in casein blocker. The membrane was developed after washing with TBS-T solution (TBS plus 0.05% Tween-20) and immersing in chemiluminescent reagents.

The foregoing results were confirmed by Noggin, Noggin inhibitor, and BMP4 treatment effects upon intestinal organ cultures in vitro, as presented in electrophoresis results depicted in FIG. 6B. Noggin treatment of intestinal organ cultures resulted in increased P-PTEN, P-AKT, Tert, and β-catenin levels, in comparison to Control levels. Tert increased dramatically; however, only a slight increase in β-catenin was observed. When the Ly294002 inhibitor was combined with Noggin treatment, P-AKT levels dropped substantially; however, the remaining activator component levels were not impacted. BMP treatment resulted in lowering of the P-AKT and β-catenin levels, where β-catenin remained in the cytoplasm. Tert levels in BMP treated intestinal segments were the same as Control.

### Example 13

It was observed that the Noggin in vitro treatment activated P-PTEN expression, as shown in FIG. 14A, middle right panel. Noggin treatment also activated P-AKT expression, as shown in FIG. 14B, middle right panel; however, this activation was inhibited by Ly294002, as demonstrated in FIG. 14B, right panel. Noggin treatment activated increased β-catenin expression, where translocation from the cytoplasm and nuclear localization was observed. Finally, Noggin treatment activated Tert expression as illustrated in FIG. 14D.

Ly294002-mediated inhibition of Noggin activation of the foregoing activation pathway components was also investigated. Increased Noggin treatment-induced P-PTEN:P-AKT:Tert:β-catenin cascade levels were specifically mediated by the PI3K/AKT pathway, since the addition of the PI3K inhibitor, Ly294002 (Calbiochem, San Diego, Calif.) significantly reduced their P-AKT activation, but had little effect on P-PTEN, as shown in FIGS. 14B and 14A, respectively. As such, P-PTEN activation by Noggin was partially sensitive to Ly294002 treatment, as shown in FIG. 14A, right panel. In addition, Tert activation was inhibited by Ly294002. Inhibition of Noggin activity by Ly294002 confirmed that Noggin activates the P-PTEN:P-AKT:β-catenin:Tert pathway in ISCs, as shown in FIG. 14C, middle right panel.

In contrast to Noggin treatment effects, BMP prevented activation of P-PTEN, P-AKT, β-catenin, and Tert. BMP4 treatment yielded lower P-PTEN and P-AKT levels in comparison with control, as shown in FIGS. 14A and 14B, left and left middle panels. BMP4 treatment also resulted in lower levels of β-catenin in comparison to control, as shown in FIG. 14C, left and left middle panels. BMP4 treatment yielded Tert levels that were equivalent to the control.

Immunohistochemical staining of ISCs revealed that Noggin induced nuclear-accumulation of β-catenin in ISCs, while Ly294002 inhibited this relocalization. This observation is consistent with a report that Noggin activates, and also has a synergistic regulation with the Wnt signal on the TOPFLash report gene mediated by the β-catenin-Tcf complex.

Thus, Noggin binding to the Bmpr1a receptor in vitro resulted in down-stream expression of activated P-PTEN, AKT, β-catenin, and Tert. The Noggin signal released BMP inhibition of ISCs, through a cascade of increased levels of activated P-PTEN, P-AKT, β-catenin, and Tert, resulting in stimulation of proliferation in the ISC population necessary to regenerate lost intestinal epithelial cells in the Wt intestine. As such, Noggin competes with BMP for Bmpr1a receptors on ISCs to activate the P-PTEN pathway. These ISC findings confirm 1) the antagonistic role of Noggin on BMP signaling; 2) the regulation of BMP/Noggin on AKT through the PTEN/PI3K pathway; and, 3) the regulation of β-catenin and Tert by AKT.

### Example 14

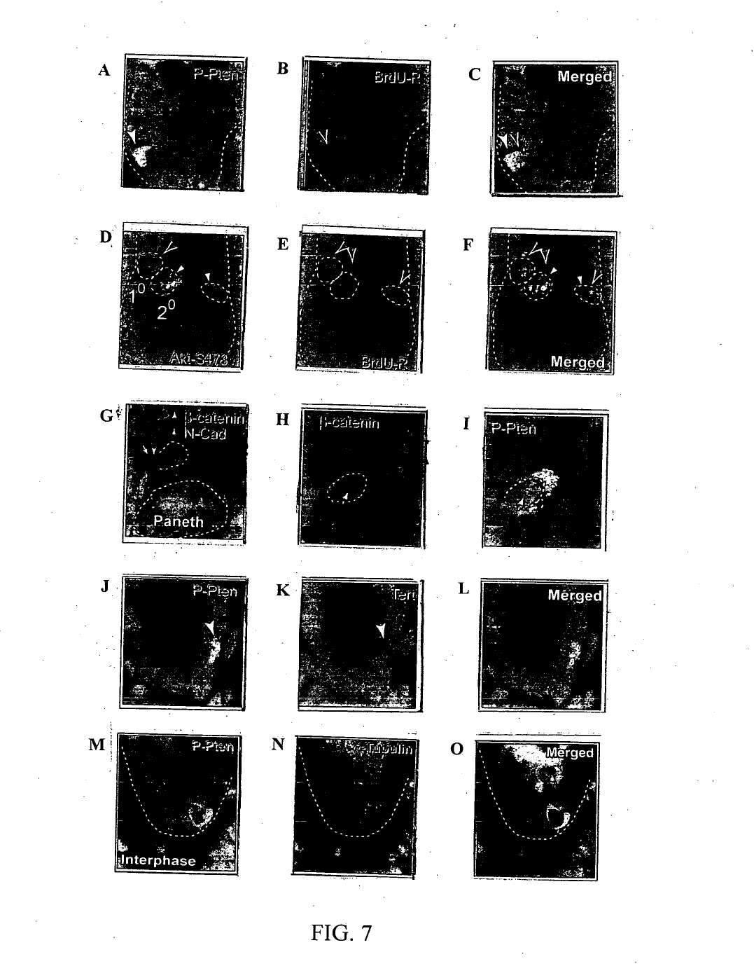

BrdU co-staining with P-PTEN, AKT-S473, Tert, and α-Tubulin was examined in ISCs to investigate the symmetry or asymmetry of cell division, as shown in FIGS. 7A-7F. Note that the BrdU shows the presence of the ISC. This relates to tumor formation in the proliferation zone.

Pups were subcutaneously injected with BrdU (10 mg/kg body weight) twice a day for 2 days. Intestinal specimens were collected 8 days after BrdU administration. BrdU in situ staining was performed using a BrdU staining kit (Zymed Laboratories Inc.) following the manufacturer's instructions. Eight days after mice were labeled with BrdU, co-staining was performed for P-PTEN, AKT-S473, Tert, and α-Tubulin markers.

P-PTEN and BrdU co-staining in Wt cells is shown in FIGS. 7A-7C. BrdU/P-PTEN marker co-staining was performed to characterize the division process. P-PTEN appeared as green, and BrdU-R appeared as red staining. P-PTEN distribution was polarized, where this marker typically appears on the adjoining surface of the ISC that attaches to the mesenchymal cell. This polarized distribution suggests that P-PTEN is important for determination of the physical orientation of division. As discussed previously, BMP signaling controls PTEN signaling, therefore, BMP is also likely involved in orientation of division.

AKT-S473 co-staining with BrdU-R is depicted in FIGS. 7D, 7E, and 7F. Both primary (1°) and secondary (2°) dividing BrdU stained cells (red) were co-stained with AKT-S473 (green), as shown in FIG. 7D. This co-staining pattern showed that in addition to P-PTEN, AKT was also present in proliferating ISCs. Both P-PTEN and AKT-S473 were detected in the cells that specifically retained the integrated BrdU, a feature characteristic of ISCs, as shown in FIGS. 7C and 7F. Co-staining of AKT-S473 and P-PTEN in the ISCs, which retain BrdU, revealed their characteristic asymmetric division pattern. The retained BrdU signal was seen in two dividing cells, as shown in FIG. 7D: one 1° mother cell aligned with other epithelial cells and maintaining contact with mesenchymal cells, and the other 2° daughter cell appeared perpendicular to the 1° cell-mesenchymal cell interface. Attachment to mesenchymal niche cells indicated that the 1° cell was the parent ISC mesenchymal cell that produced BMP. Therefore, the 2° cell was the daughter cell, which possessed a stronger AKT-S473 signal and was in a perpendicular position to the 1° cell and the niche interface.

β-catenin was asymmetrically localized, and formed an adherens complex with N-cadherin, at the interface between the arrested ISC and mesenchymal cell, as shown in FIG. 7G. Nuclear-accumulation of β-catenin was seen in P-PTEN-positive ISCs, as shown in FIG. 71. It was concluded that, when the stem cell was in the arrested state, β-catenin was present in the membrane-associated form, with N-cadherin. When PTEN became inactivated, β-catenin accumulated in the nucleus resulting in activation of stem cell division.

Tert co-stained with P-PTEN, is shown in FIGS. 7K and 7L. Tert co-staining with P-PTEN is shown in FIGS. 7J-7L. Tert staining was faint because nuclear staining was diminished, resulting in a speckling effect. This result suggests that asymmetric division occurred horizontally and perpendicular to the mesenchymal niche cell/ISC interface, as shown in FIG. 7L. This unexpected finding directly contradicts the prevailing, long-felt scientific opinion that ISC division is vertical. Thus, detection of P-PTEN, AKT-S473, and Tert markers in ISCs was specifically confirmed. Asymmetry and symmetry of cell division was illustrated in the ISC population.

α-Tubulin co-staining with P-PTEN is shown in FIGS. 7M-7O. P-PTEN stains ISCs, and α-Tubulin staining was specific for spindles used in separation of chromosome sets in dividing cells. A crypt with two dividing cells, where P-PTEN appeared at the poles, and α-Tubulin was present at the center of ISCs, is shown in FIG. 7O. As previously, an asymmetrical pattern of cell division was observed for Wt tissue. In the above Wt tissues, division was shown to be asymmetrical. This further illustrated the presence of P-PTEN in dividing cells.

Both P-PTEN and AKT-S473 were detected in the cells that specifically retained the integrated BrdU (BrdU-R) label, as characteristic features of ISCs, as shown in FIGS. 7A-7C and FIGS. 7D and 7E. During early prophase of ISCs, P-PTEN was found to be enriched on the side of the ISCs adjacent to mesenchymal cells. α-tubulin co-localized with a lower level of P-PTEN staining on the opposite side, as shown in FIGS. 7M and 7N. While undergoing ISC division, cellular localization of P-PTEN was polarized and restricted to the two poles of the dividing cells in the crypts of Wt mice, as shown in FIGS. 7M and 7O.

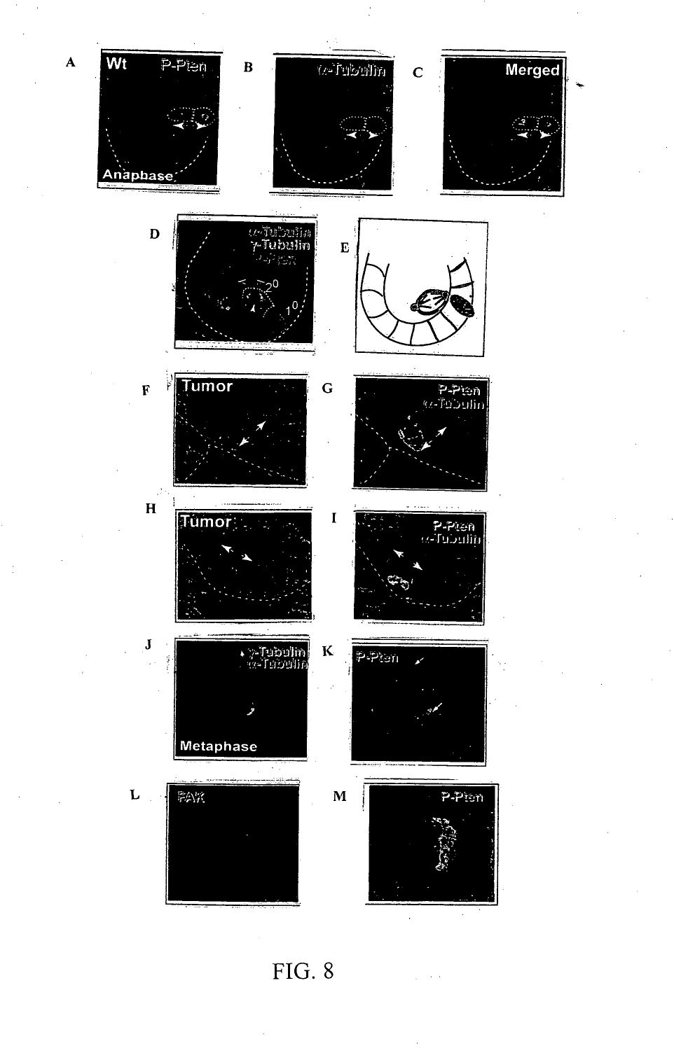

Asymmetric and symmetric division patterns of ISCs, revealed by co-staining of P-PTEN with BrdU-R in Wt and Bmpr1a mutant intestines, can also be seen in FIGS. 7D-7F and FIGS. 8F-8G. Whether the Wt asymmetric division pattern is disrupted in the Bmpr1a mutant intestine was investigated; however, an increased number of ISCs was found, as shown in FIGS. 8F-8G and FIGS. 8H-8I. Unexpectedly, multiple ISCs doublets were observed that were positive for either P-PTEN or AKT-S473 in multiple crypts, as shown in FIGS. 8D-8E and FIGS. 8F and 8G. In addition, these duplicated ISCs were each able to attach to mesenchymal cells in the Bmpr1a mutant intestine, as shown in FIGS. 8F and 8G, showing unequivocally that symmetric stem cell division did indeed occur in some ISCs when the BMP signal is blocked. α-Tubulin co-staining with P-PTEN of murine tumors in Bmpr1a mutant mice is shown in FIGS. 8F and 8H. Symmetric division was observed in tumor cells in FIGS. 7M-7O and FIGS. 8H-8I, where horizontal spindle formation occurs. However, both symmetric and asymmetric division in tumor cells was observed, in contrast to only the asymmetric division observed in normal intestinal cells.

After ISC division, the 2° daughter cell further divided in the crypt, as shown in FIG. 8D. In further support of this observation, P-PTEN was co-stained with α-tubulin and γ-tubulin, components of the spindle, permitting visualization of the orientation of mitotic cells relative to each other, as shown in FIG. 8D. In the 2° cell, α-Tubulin is visible at the poles of a dividing cell, where white arrows indicate the outward ends of the mitotic spindles and red arrows indicate the horizontal plane of division.

In contrast to the observation of solely asymmetric cell division of ISCs in the Wt intestine, as shown in FIGS. 7D-7E and FIGS. 8A-8C, both asymmetric and symmetric stem cell division were seen in the mutant tumor region, as shown in FIGS. 8F-8G and FIGS. 8H-8I. This tumor tissue finding confirmed that when the BMP signal is blocked the orientation of division was randomized.

Co-staining of P-PTEN with α-tubulin (for spindle) and γ-tubulin (for centrosome) revealed that P-PTEN was located on the pole sides and adjacent to centrosomes of dividing ISCs, as shown in FIGS. 8J and 8K. This observation suggested that P-PTEN and the underlying complex were involved in regulating orientation of the spindle through the centrosome in mitotic ISCs, as shown in FIG. 8J. This function is potentially mediated by focal adhesion kinase (FAK), which is involved in microtubule organization, FAK co-localizes with P-PTEN, as shown in FIGS. 8L and 8M.

Consistent with the foregoing findings, it was concluded that (1) daughter stem cells derived from asymmetric division, such as the 2° cells seen in FIG. 11C, are committed to proliferation and differentiation as a result of loss of contact with mesenchymal (niche) cells; and (2) the daughter stem cells derived from symmetric division still maintain their full potential to give rise to new crypt/villus units. Thus, tumor formation results from symmetric division of ISCs which triggered crypt fission. During this process, an increase was observed both in the number of crypts and in the proliferation of progenitor cells. This process was further characterized by unbalanced lineage commitment and resistance to programmed cell death. All these foregoing events account for tumorigenicity, which appeared as a direct consequence to the disruption of aspects of the zonal regulation imposed by the BMP signal.

The permanent block of the BMP signal in the Bmpr1a mutant mouse led to inactivation of PTEN and the loss of the mechanisms controlling asymmetric ISC division. In the normal Wt villus, daughter cells derived from asymmetric ISC division undergo proliferation and differentiation due to changes in their environment, as shown in FIGS. 8D and 8E. In contrast, daughter cells derived from symmetric ISC division in Bmpr1a mutants retain their full capability to give rise to crypt/villus units, which lead to the generation of multiple crypts in the tumor, as schematically illustrated in FIGS. 11C-11D. Proliferation in mutants is characterized by asymmetric and symmetric division, as well as an increase in P-PTEN and P-AKT. Ultimately, this leads to crypt fission and tumor formation.

### Example 15

After investigating the proliferation zone of the intestinal villi in the previous examples, the differentiation zone was examined. The investigation focused on whether epithelial cell differentiation is affected in Bmpr1a mutant intestines in comparison to Wt intestines. ISCs differentiate into columnar (C), mucosal (M), and neuroenteroendocrine (endocrine) progenitors, as illustrated diagrammatically in FIG. 1I A. The C progenitors produce enterocytes, which have an absorptive function. The M progenitors give rise to mucin-producing goblet cells and paneth cells.

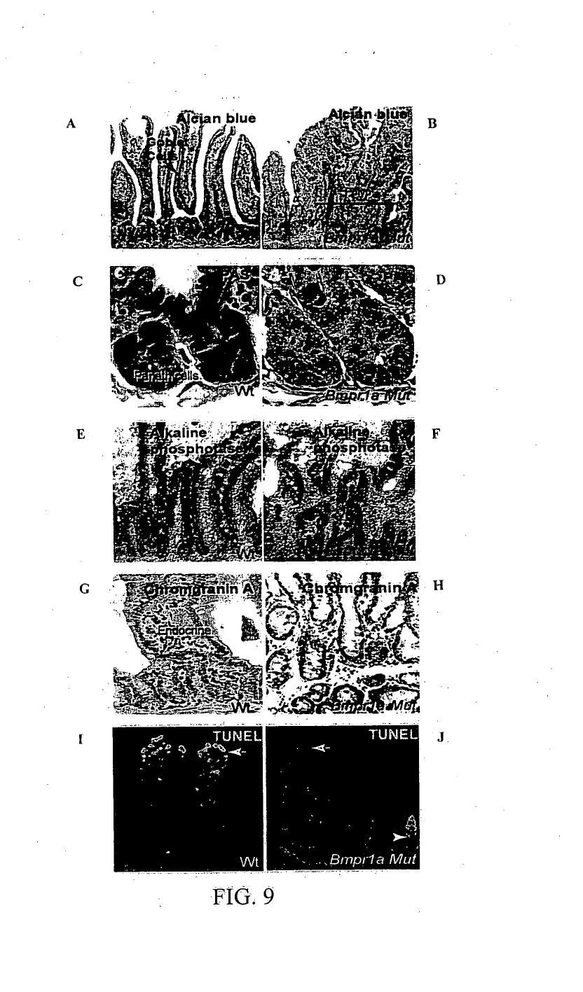

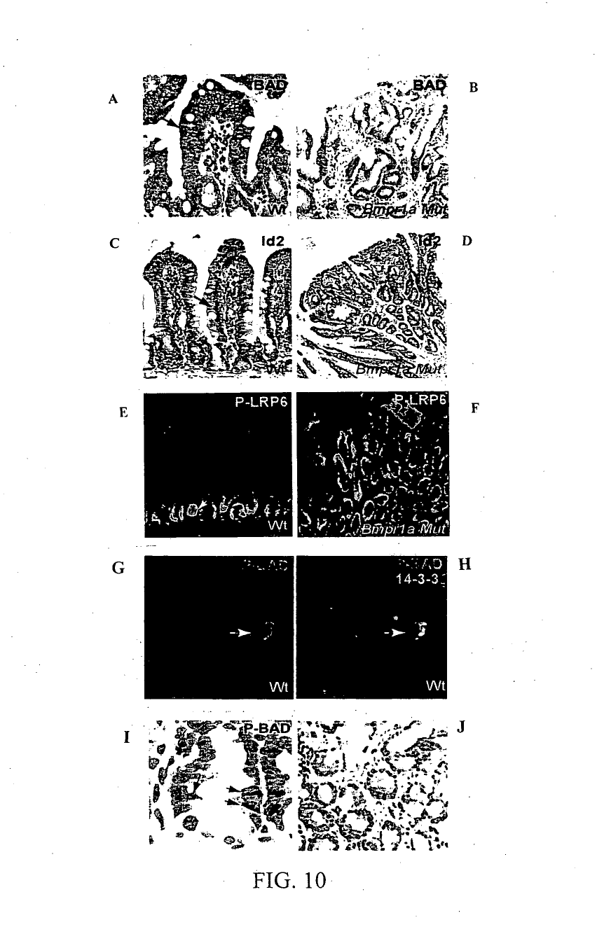

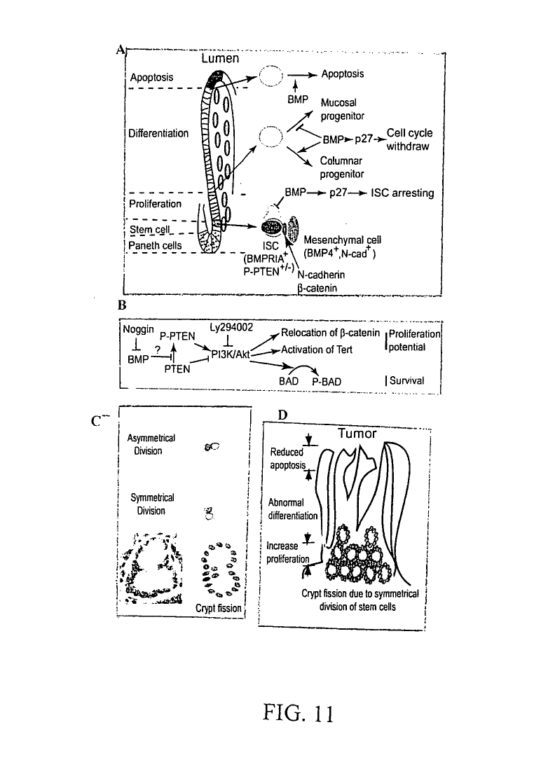

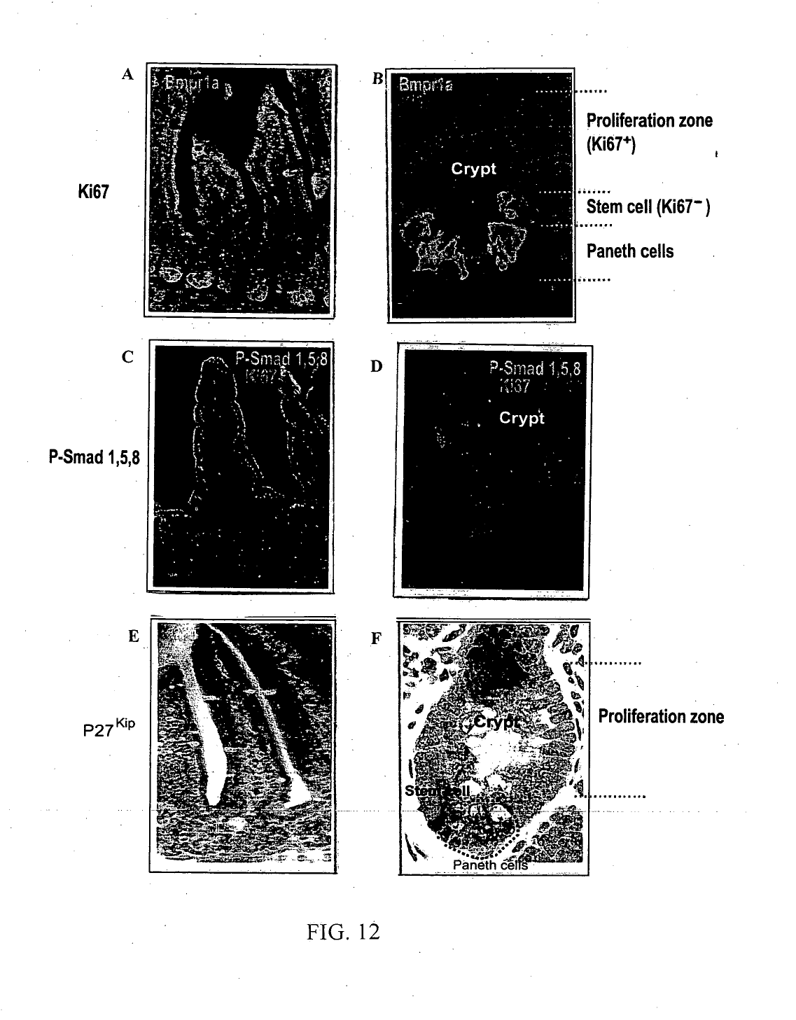

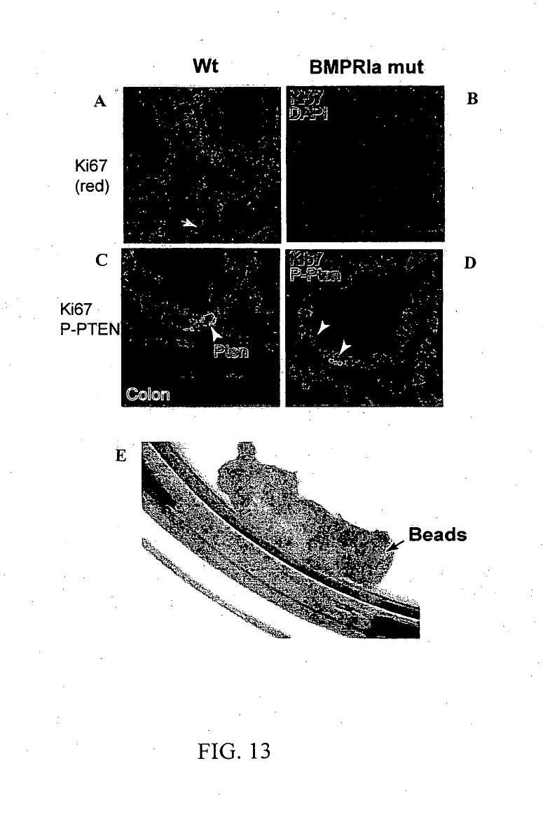

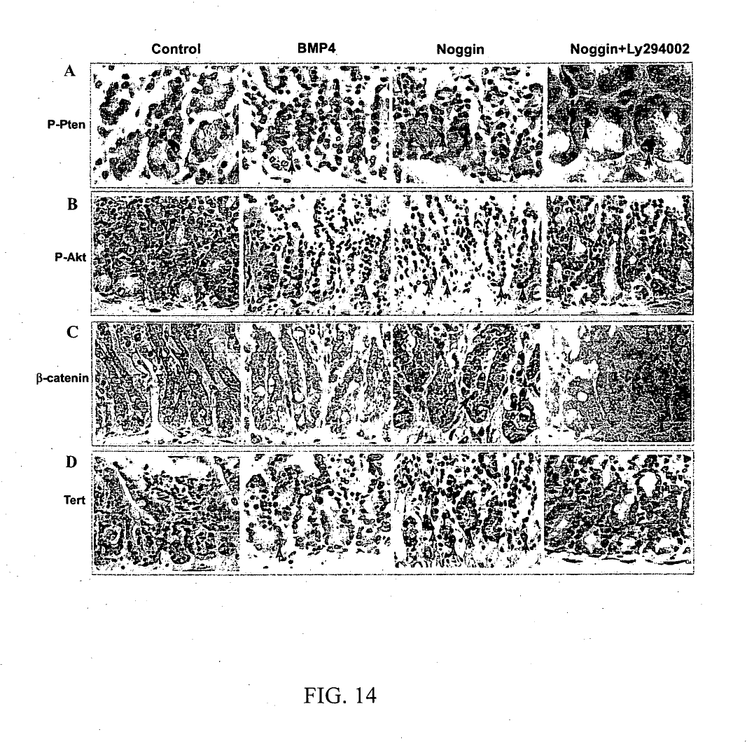

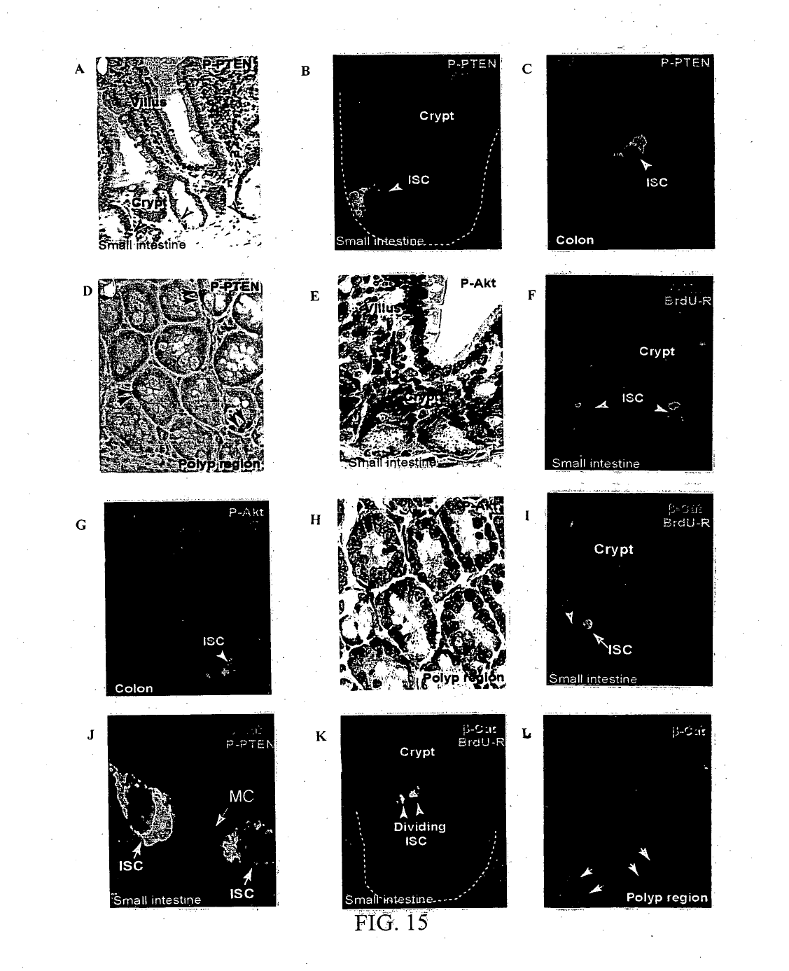

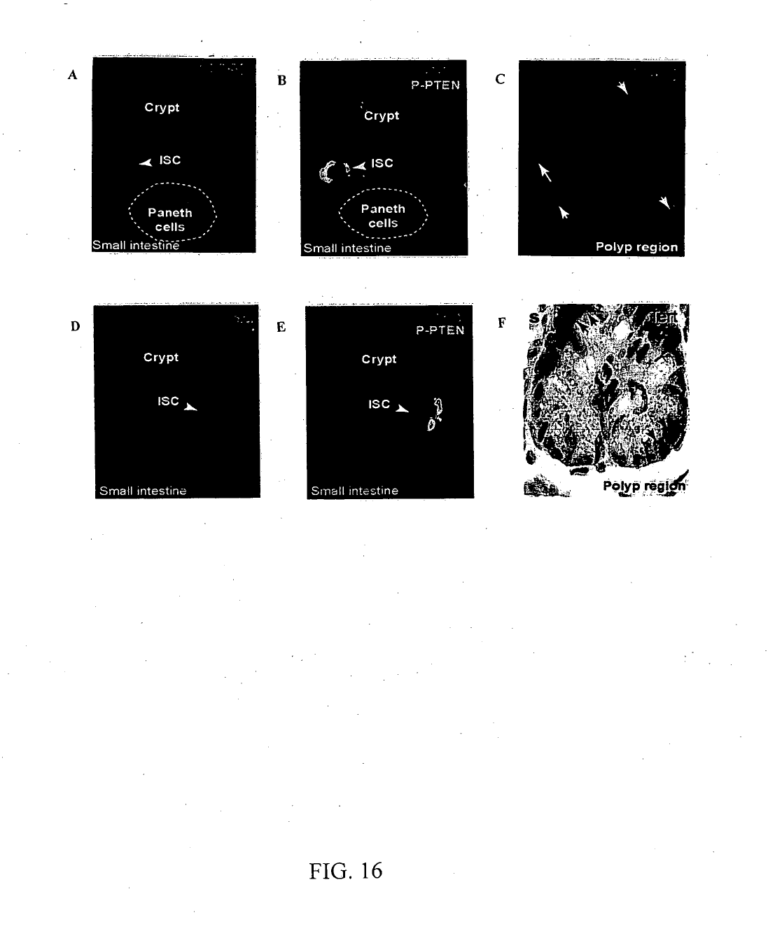

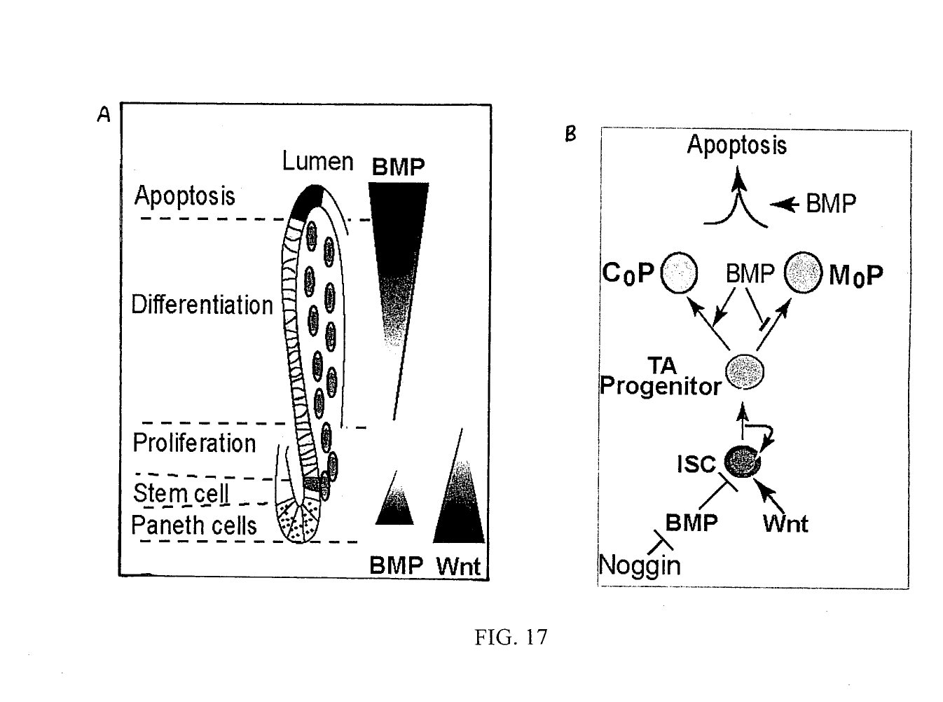

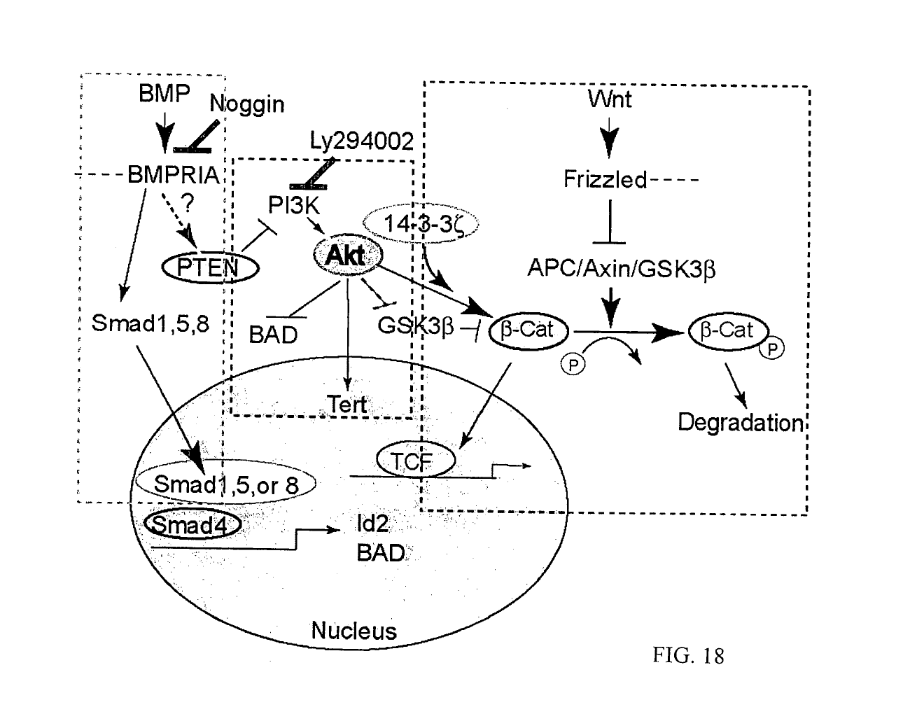

Goblet cells secrete mucus, used in digestion of food for absorption of nutrients through the intestinal villi. Goblet cells, stained with Alcian blue, are shown in the Wt mouse, as depicted in FIG. 9A. In the Bmpr1a mutant mouse, the intestinal sections exhibited a 3-4 fold increase in goblet cells, in tumorous cysts, as shown in FIG. 9B.

Similarly, paneth cells at the bottom of the crypt increased in cell number about 1.5 to 2.0 fold in mutant mice in comparison to Wt mice, in PAS stained sections, as shown in FIGS. 9C and 9D, respectively. In contrast, enteroendocrine cells, stained with the Anti-ChromgrinA marker, as depicted in FIGS. 9G and 9H, showed no difference between Wt and tumor tissue, respectively. Similarly, the alkaline phosphatase marker, as shown in FIGS. 9E and 9F, yielded no difference in Periodic Acid-Schiff (PAS) staining of villi in Wt in comparison to tumor tissue. The number of enterocytes, detected by alkaline phosphatase, was decreased in the Bmpr1a mutant mice, as shown in FIG. 9F.

These results indicate that when the BMP signal is blocked, epithelial differentiation is impaired and lineage commitment is unbalanced, resulting in an excess of mucosal cells at the expense of cells committed to become enterocytes. This accounts for the significant increase in mucin-accumulated cysts in the abnormal intestines.

To further determine which downstream component of BMP signaling might be involved in the regulation of epithelial lineage commitment, the expression pattern of Id2 was analyzed, which is reported to be a target gene of BMP4 signaling. Expression of Id2 was high in intestinal villi (FIG. 10C) but low in the crypts and significantly down-regulated in the polyp region (FIG. 10D), displaying a similar pattern to that of P-Smad1,5,8. This suggests that Id2 is involved in the regulation of epithelial lineage commitment in response to BMP signaling.

Wnt signaling favors crypt fate, which is confirmed by the staining of the activated (phosphorylated) form of LRP6 (P-LRP6). LRP6, a co-receptor for Wnt, which becomes phosphorylated upon Wnt binding, is predominantly expressed in crypts (FIGS. 10E-10F). FIG. 10F shows an increase in crypts in the mutant. Thus, BMP signaling promotes villus fate, favoring epithelial differentiation, which is opposite to Wnt signaling, which promotes proliferation of progenitor cells in crypts.

It was concluded that the BMP signal was important for epithelial cell differentiation and that BMP was directly involved in determination of lineage fate, as depicted in FIGS. 9A-9H. Results suggested that the BMP signal inhibited the differentiation of mucin-producing cells, such as paneth and goblet cells. Taken together, these results revealed that the BMP signal plays a critical role in determining cell fate by favoring columnar over mucosal lineages. When the BMP signal was blocked in the Bmpr1a mutant intestines, epithelial differentiation was impaired and lineage commitment was unbalanced, resulting in an excess of mucosal cells at the expense of cells committed to becoming enterocytes. This accounted for the significantly increased appearance of mucin-producing cysts in the abnormal small and large intestines, as shown in FIGS. 3C-3F. However, enterocyte differentiation in the Bmpr1a mutant mouse was not affected. Support for the foregoing conclusions was found in tumors and polyps of Bmpr1a mutant mice. Numerous goblet cells in the tumorous cysts of mutants secreted increased mucin levels in comparison with goblet cells of normal Wt mice.

It was observed that differentiation was partially inhibited in the tumor region of Bmpr1a mutant mice. In contrast, in normal Wt tissue, for cells residing in the differentiation zone, the existence of a low to intermediate BMP activity level in this zone was conducive to differentiation. In addition, this Wt BMP activity level also directly impacted lineage fate determination by favoring columnar lineage fate over mucosal fate.

### Example 16

The involvement of BMP signaling in inducing epithelial cell apoptosis was analyzed. As background information, intestinal homeostasis depends upon both cell proliferation and cell death in equilibrium. In a rapidly renewing intestinal system, cells are constantly lost from the villi into the gut lumen and are replaced at an equal rate by proliferation of cells in the crypts. The BMP signal is implicated in inducing epithelial cell death since up-regulation of Smad5 mediates apoptosis of gastric epithelial cells. Apoptotic features in the intestines of Wt control mice were compared to Bmpr1a mutant mice assayed by the presence of Bc 12-associated death promoter (BAD), a pro-apoptotic molecule. BAD is a preaptotic molecule triggering cell death through inhibition of B cell 2 and B cell XL. Blocking B cell 2 induces cell death. The apoptosis zone is located at the tips of the villi.

Apoptotic features in the intestines of Wt control mice were compared to Bmpr1a mutant mice by both the TUNEL assay and the presence of apoptotic molecules, including BAD, which inhibits Bcl2 family members. The TUNEL assay showed that cells in the tip of villi of Wt and in normal regions of mutant intestine are apoptotic, while cells at the tip of polyps are resistant to apoptosis (FIGS. 9I-9J). This was consistent with high levels of BAD detected in cells located in the tip of the villi (the apoptotic zone) in the Wt intestine (FIG. 10A); however, BAD expression was rarely seen in the polyp region of the Bmpr1a mutant intestines (FIG. 10B). Thus, the epithelial cells at the tip of the villi are resistant to apoptosis when the BMP signal is blocked.

It was observed that BAD staining was high in ISCs, where BMP activity was also high. The anti-P-BAD antibody (P-BAD: BAD-S136) was utilized and found that it was phosphorylated in the ISCs, evidenced by co-staining with 14-3-3ζ (FIGS. 10G-10H). This is consistent with an AKT function in priming BAD through phosphorylation on S136, to allow its binding to 14-3-3, and inhibiting the pro-apoptotic function of BAD. Thus, AKT also promotes a survival signal in the ISCs.

It was queried whether BAD existed in active or inactive (phosphorylated) forms. The anti-P-BAD antibody (P-BAD: BAD-S136) was utilized to show that BAD was phosphorylated in the ISCs and the immediate downstream progenitors, as shown in FIG. 101. In contrast, a much weaker P-BAD (and BAD) signal was detected in the tumor region of the Bmpr1a knock-out mutant, as shown in FIG. 10J. This finding is consistent with the function of AKT-S473, which can phosphorylate BAD at the site of S136 to inhibit its pro-apoptotic function. In keeping with this function, AKT provides a survival signal to the ISCs to protect them from apoptosis.

Thus, in the apoptotic zone of Wt mice, the highest BMP activity induced cell apoptosis, through increased BAD activity, as shown in FIG. 10H; however, in Bmpr1a mutants, the cells in the apoptotic zone were resistant to apoptosis due to the loss of BAD signaling resulting from conversion to P-BAD. Correspondingly, in tumor regions of mutant mice, P-BAD levels rise, as shown in FIG. 10J, and BAD levels drop, as shown in FIG. 1-B.

### Example 17

Bmpr1a mutant and Wt antigens to be prepared for immunization and to be used as standards in immunoassays include Bmpr1a Wt and mutant polypeptide whole molecule and polypeptide fragments. In addition, the corresponding Bmpr1a-derived nucleic acid molecules to the aforementioned polypeptide molecules can be produced as antigens for immunizations and standards.

Goat and rabbit polyclonal antibodies and mouse monoclonal antibodies to the Bmpr1a-derived Wt and mutant polypeptide are prepared by methods that are known to those of skill in the art. E. Harlow and D. Lane, Antibodies: A Laboratory Manual, Cold Spring Harbor Laboratory, New York, 1988.

Once monoclonal and polyclonal antibodies to Bmpr1a-derived polypeptide and nucleic acid molecules have been made, they can be utilized in immunodiagnostic kit assays for the detection and quantitation of the Bmpr1a-derived molecules. As such, immunodiagnostic kits containing anti-Bmpr1a, anti-BMP, anti-Noggin, anti-PTEN, anti-P-PTEN, anti-AKT, anti-P-AKT, anti-Tert, anti-β-catenin, anti-Ki67, anti-p27, anti-Smad1,5,8, anti-tubulin, anti-Chromgrin A, anti-BAD, anti-PBAD, and anti-FAK antibodies can be utilized for the detection and quantitation of individual markers associated with ISC and intestinal cell activation, proliferation, differentiation, apoptosis, and polyposis. These foregoing kits may be used either in vitro or in vivo.

Bmpr1a, BMP, and Noggin immunodiagnostics test kits can be made and used by the following procedure: mutant and Wt Bmpr1a, Wt BMP, and Wt Noggin polypeptides from intestinal cells can be detected, isolated, and amplified by standard molecular biological techniques. The foregoing polypeptide molecule antigens are then injected into mice, rabbits, and goats to make monoclonal and polyclonal antigen-specific antibodies. For monoclonal antibody production, murine monoclonal antibodies to Bmpr1a, BMP, and Noggin polypeptides can be isolated and purified from supernatants of cultured hybridoma cells by known fusion, hybridoma selection and cultivation methodologies in selective medium. For polyclonal antibody production, the foregoing polypeptide antigens can be injected into goats and rabbits in complete Freund's adjuvant, then boosted several times to produce secondary antibody responses.

The antibodies are then used to form a sandwich 96 well microtiter plate immunoassay can be made for detection and quantitation of Bmpr1a, BMP, and Noggin in intestinal tissue. Polyclonal anti-Bmpr1a, BMP, and Noggin polypeptide antibodies can be coated onto separate 96 well microtiter plates (1 mg/ml, 100 μl per well) in carbonate coating buffer, then blocked with blocking buffer containing BSA and stored for later use. Serial two-fold dilutions of intestinal tissue extracts from either Bmpr1a mutant or Wt mice are added to the wells. Similarly, two-fold dilutions of purified intestinal stem cells and other cell populations, isolated by FACS sorting techniques, can be added to wells. In separate wells, serial two-fold dilutions of Bmpr1a, BMP, and Noggin standards are added, incubated for 2 hours at 37° C., then rinsed in BSA wash buffer.

Alkaline phosphatase labeled mouse monoclonal antibodies to Bmpr1a, BMP, and Noggin are then added to wells, incubated, and washed. 4-methyl-umbelliferyl phosphate (MUP) is added as substrate, and the fluorescence emission in each well measured in a fluorescence microtiter reader. By comparing the quantitative amount of fluorescence in unknown vs. standard, the amount of Bmpr1a, BMP, and Noggin can be measured and compared among Bmpr1a mutant and Wt tissues.

### Example 18

Bmpr1a mutant and Wt intestinal tissue can be fixed and stained with fluorescein isothiocyanate (FITC) labeled mouse monoclonal antibodies for Bmpr1a, BMP, and Noggin. Localized fluorescence can be detected and measured on or in intestinal cells and cell populations by fluorescence microscopy. For example, Bmpr1a, BMP, and Noggin can be detected on ISCs and other intestinal cell populations in villi of small and large intestines. In addition, the amount of fluorescence per cell can be visually assessed by a 0, 1+ to 4+ semi-quantitative cell scoring system. By a similar procedure, BMP and Noggin associated with ISCs and other intestinal cell populations can be visually detected and quantitated in intestinal tissues. Tissue sections from small and large intestine can be stained.

Specifically, a mouse monoclonal antibody can be made that is directed against Wt Bmpr1a polypeptide encoded by a Bmpr1a gene containing intact Exon 2, and this antibody should be nonreactive against Bmpr1a mutant polypeptide lacking the Exon 2-encoded region. Such a murine monoclonal antibody, if labeled with FITC, would stain Wt ISCs but not Bmpr1a mutant ISCs. As such, Wt ISCs will fluoresce green, but Bmpr1a mutant ISCs will not. Similarly, a mouse monoclonal antibody might also be made that is directed against a Bmpr1a mutant polypeptide lacking the Exon 2-encoded region. This antibody, if labeled with rhodamine, should react with Bmpr1a mutant polypeptide, but not with Wt Bmpr1a polypeptide. Thus, clonally mutant villi will stain red, and Wt villi will stain green utilizing the foregoing immunofluorescent reagents.

### Example 19

Kit components for detection and quantitation of Bmpr1a Wt and mutant polypeptides and fragments are described. Immunodiagnostic methodologies utilized in these kits are modifications of general and specific principles well known in the art. E. Harlow and D. Lane, Antibodies: A Laboratory Manual, Cold Spring Harbor Laboratory, New York, 1988, and E. T. Maggio, Ed., Enzyme-Immunoassay, CRC Press, Florida, 1980.

Sandwich enzyme immunoassay (EIA) kit components are as follows: 96-well microtiter plates coated with anti-Bmpr1a antibody directed against Wt Bmpr1a molecules, 96-well microtiter plates coated with anti-Bmpr1a antibody directed against mutant Bmpr1a molecules, diluent buffer, Wt and mutant Bmpr1a standards, horseradish peroxidase (HRP)-conjugated mouse anti-Bmpr1a antibody, ortho-phenylenediamine (OPD) substrate solution, containing H₂O₂, and 2N sulfuric acid stop solution.

In the sandwich EIA procedure, Triton X-100 extracts from homogenized mutant Bmpr1a murine intestinal tissue in phosphate-buffered saline (PBS) are serially two-fold diluted in PBS in wells of the Wt Bmpr1a plates and wells of the mutant Bmpr1a plates. Mutant Bmpr1a small or large intestine tissue can be obtained from PolyI:C-induced post-excision mutant mice. Similarly, extracts from Wt Bmpr1a murine intestinal tissue are diluted into wells of Wt Bmpr1a and mutant Bmpr1a plates. Serial two-fold dilutions of purified Wt and mutant Bmpr1a polypeptide preparations are used as quantitative control standards in each set of microtiter plates. By spectrophotometrically measuring the colorimetric difference in OPD substrate absorbance at 405 nm in a microtiter EIA reader in Bmpr1a mutant as compared to Bmpr1a Wt intestinal tissue, the percentage of Bmpr1a mutation-containing villi can be quantitatively assessed in an unknown Bmpr1a mutant tissue.

Competitive enzyme immunoassay (EIA) kit components are as follows: 96-well microtiter plates coated with mutant Bmpr1a molecules, 96-well microtiter plates coated with Wt Bmpr1a molecules, diluent buffer, Bmpr1a Wt and mutant standards, horseradish peroxidase (HRP)-conjugated mouse anti-Bmpr1a antibody, ortho-phenylenediamine (OPD) substrate solution, containing hydrogen peroxide (H₂O₂), and 2N sulfuric acid stop solution. The label on the antibody can also be a radioactive, colorimetric, fluorometric, bioluminescent, or chemiluminescent label, as is known in the art.

In the competitive EIA procedure, intestinal tissue extracts in PBS buffer are serially two-fold diluted into wells of mutant Bmpr1a microtiter plates and also wells of Wt Bmpr1a microtiter plates. Serial two-fold dilutions of Wt and mutant Bmpr1a standards are also made as references. After incubation and wash, HRP-conjugated anti-Bmpr1a antibody and OPD substrate are added sequentially. By measuring inhibition of binding by Bmpr1a mutant intestinal tissue extracts of colorimetric signal at 405 nm in comparison with Wt intestinal tissue extracts, the percentage of mutant Bmpr1a in the intestinal tissue can be quantitatively assessed.

### Example 20

Immunodiagnostic kits for detection and quantitation of PTEN-PI3K-AKT cascade components (i.e., P-PTEN, PTEN, P-AKT, PI3K, 14-3-3ζ, Telomerase, Tert, GSK3β, β-catenin) in Wt and Bmpr1a mutants are described. Immunodiagnostic methodologies utilized in these kits are modifications of general and specific principles well known in the art. E. Harlow and D. Lane, Antibodies: A Laboratory Manual, Cold Spring Harbor Laboratory, New York, 1988, and E. T. Maggio, Ed., Emzyme-Immunoassay, CRC Press, Florida, 1980.

Polyclonal and monoclonal antibodies to specified PTEN-PI3K-AKT cascade antigens (P-PTEN, PTEN, P-AKT, AKT, PI3K, 14-3-3ζ, Telomerase, Tert, GSK3β, β-catenin, P-Smad1,5,8, Smad1,5,8, and BAD antigens) from Wt or Bmpr1a mutant organisms can be made by immunization of rabbits and mice, respectively, with antigen in Complete Freund's Adjuvant (CFA). After one month, animals are boosted with antigen in IFA, sera obtained and screened for antibody-specific binding activity by standard enzyme immunoassay (EIA) methods. Alternatively, commercial antibodies from Cell Signaling Technology can be used as follows: P-PTEN (Ser380, #9551), P-PTEN (Ser380/Thr382/383, #9554), PTEN (#9556, 9552), PI3K (4292, 4252, 4254), P-AKT (#4051, 9271, 9277, 9275, 2968, 5102), AKT (#2966, 5116), GSK-3p (#4042, 9332, 9331, 9551, 9554), P-BAD (#9290), and BAD (#9292). Tert antibodies (NB 100-141) were obtained from Novus.

Sandwich enzyme P-PTEN-PI3K-AKT (PPA) cascade immunoassay (EIA) kit components are as follows: 96-well microtiter plates coated with antibody directed against one of the Wt PPA cascade molecules, 96-well microtiter plates coated with anti-PPA antibody directed against mutant PPA cascade molecules, diluent buffer, Wt and mutant Bmpr1a standards, horseradish peroxidase (HRP)-conjugated anti-PPA antibody, ortho-phenylenediamine (OPD) substrate solution, containing H₂O₂, and 2N sulfuric acid stop solution.

In the sandwich EIA procedure, Triton X-100 extracts from homogenized mutant Bmpr1a murine intestinal tissue in phosphate-buffered saline (PBS) are serially two-fold diluted in PBS in wells of the Wt PPA cascade antigen plates and wells of the mutant Bmpr1a PPA cascade antigen plates. Mutant Bmpr1a small or large intestine tissue can be obtained from PolyI:C-induced post-excision mutant mice. Similarly, extracts from Wt Bmpr1a murine intestinal tissue are diluted into wells of Wt and mutant Bmpr1a plates. Serial two-fold dilutions of purified Wt and Bmpr1a mutant PPA cascade-containing polypeptide preparations are used as quantitative control standards in each set of microtiter plates. The colorimetric difference in OPD substrate absorbance at 405 nm can be measured in a microtiter EIA reader in Bmpr1a mutant as compared to Wt intestinal tissue.

Competitive PPA cascade enzyme immunoassay (EIA) kit components are as follows: 96-well microtiter plates coated with PPA cascade molecules from Bmpr1a mutants, 96-well microtiter plates coated with Wt PPA cascade molecules, diluent buffer, Wt and mutant PPA cascade standards, horseradish peroxidase (HRP)-conjugated mouse anti-PPA cascade molecule antibody, ortho-phenylenediamine (OPD) substrate solution, containing hydrogen peroxide (H₂O₂), and 2N sulfuric acid stop solution. Alternatively, the label on the antibody can be a radioactive, colorimetric, fluorometric, bioluminescent, or chemiluminescent label, as is known in the art.

In the competitive EIA procedure, intestinal tissue extracts in PBS buffer are serially two-fold diluted into wells of mutant Bmpr1a microtiter plates and also wells of Wt Bmpr1a microtiter plates. Serial two-fold dilutions of Wt and mutant Bmpr1a PPA cascade standards are also made as references. After incubation and wash, HRP-conjugated anti-PPA cascade antibody and OPD substrate are added sequentially.

### Example 21

An immunoprecipitation protocol and subsequent Western Blot protocol are described for analysis and characterization of various Bmpr1a-derived proteins and polypeptide molecules. Western blot kits based on the methodology described herein may also be produced.

Western blot kits can contain the following components: Bmpr1a-derived protein and polypeptide molecule standards, primary goat antibody against Bmpr1a, secondary alkaline phosphatase-conjugated anti-goat antibody, blocking buffer, diluent buffer, and substrate development solution.

The immunoprecipitation protocol involves a technique for separation of Bmpr1a-derived polypeptide molecules from whole cell lysates or cell culture supernatants. Bmpr1a-derived polypeptide molecules may be Wt or mutant molecules; and these molecules may be obtained from mammalian cell cultures (e.g., ISCs), mammalian tissue (e.g., intestine), or bacterial cells (e.g., E. coli). After immunoprecipitation binding to anti-Bmpr1a antibody and separation of these Bmpr1a-derived polypeptide molecules, the Bmpr1a molecules can be identified, biochemically characterized, and expression levels quantitated.

In initial immunoprecipitation runs, approximately 5-10 μg of anti-Bmpr1a-derived polypeptide molecule antibody is added to an Eppendorf tube containing the cold precleared lysate containing Bmpr1a polypeptides. Alternatively, antibodies recognizing an incorporated MYC tag may be utilized for these immunoprecipitations of Bmpr1a polypeptides. Reduced and nonreduced Bmpr1a-derived polypeptide molecules are prepared to run alongside prestained molecular weight standards for use on SDS-PAGE gels.

In the R&D System Immunostaining procedure, Western Blot membranes are blocked in Blocking Buffer, incubated with primary goat anti-Bmpr1a polypeptide antibody, incubated with secondary antibody (e.g., alkaline phosphatase conjugated anti-goat IgG antibody), incubated with Substrate Development solution, dried, and blocked in Blocking Buffer. Unoccupied protein binding sites on membrane are blocked by placing the membrane in Blocking Buffer on a rocker/shaker. Primary antibody (e.g., goat anti-Bmpr1a polypeptide molecule antibody) in Diluent Buffer is added to the membrane and incubated. After washing, blots are incubated with 20 mL of secondary antibody (e.g., TAGO alkaline phosphatase-conjugated rabbit anti-goat IgG antibody) in Diluent Buffer and incubated. Membranes are washed, incubated, and then Substrate Development Solution is added to membrane. Substrate development is stopped after incubation by removing Development Solution and rinsing the membrane in deionized water.

In summary, this Western blot methodology can be used to identify, biochemically and immunologically characterize, and quantitate Bmpr1a polypeptide molecules derived from Wt and/or mutants in both mammalian and bacterial cell culture systems. In addition, Western blot kits may be produced utilizing Bmpr1a-derived molecule standards, antibodies, and kit components described and utilized in the above-described methodology.

### Example 22

A Western Blot diagnostics kit is described for analysis and characterization of phosphorylated PTEN (P-PTEN) and phosphorylated AKT (P-AKT) derived proteins and polypeptide molecules. Intestinal tissue from either Wt or Bmpr1a mutant organisms is homogenized in a cocktail of 1 ml lysis buffer (100 mM Tris-HCl, pH 6.8, 2% SDS and a Roche protease inhibitor cocktail). The supernatants, containing the foregoing protein molecules of interest, are collected after centrifugation. As previously described, in initial immunoprecipitation runs, 5-10 μg of anti-P-PTEN is added to supernatants containing the desired molecules. In other runs, anti-P-AKT is added.

Protein extracts (75 μg/well) are fractionated on SDS-PAGE and transferred onto nitrocellulose membranes. The membrane was washed with TBST solution (Tris-buffered saline plus 0.05% Tween-20). In some tubes, rabbit anti-P-PTEN (#9551, Cell Signaling Technology) antibody solution is mixed with either Wt or Bmpr1a mutant intestinal tissue extracts containing cells possessing P-PTEN, PTEN, P-AKT, and AKT. In other tubes, rabbit anti-P-AKT Ser473 (#9271, #9275, Cell Signaling Technology) is mixed with Wt or Bmpr1a mutant extracts. HRP-conjugated goat anti-rabbit IgG (#7074, Cell Signaling Technology) was added, followed by luminol chemiluminescent substrate reagents (Santa Cruz). In the presence of hydrogen peroxide, HRP converts luminol to an excited intermediate dianion that emits light. Collected light exposes X-ray film, where the intensity of the exposure corresponds semiquantitatively with amount of P-PTEN or P-AKT present. The phospho-specificity of the antibodies was established by treating the membrane with or without calf intestine alkaline phosphatase after Western blot transfer.

Alternative, polyclonal anti-P-PTEN and P-AKT antibodies can be made by immunizing rabbits with synthetic P-PTEN or P-AKT polypeptide residues coupled to keyhole limpet hemocyanin carrier (KLH) in Complete Freund's Adjuvant (CFA), such as those surrounding Ser380 of PTEN, Ser 473 or Thr308 of AKT. Antiserum from immunized rabbits can be screened for selective binding against P-PTEN or P-AKT, and for absence of binding to nonphosphorylated PTEN and AKT. Monoclonal antibodies to P-PTEN or P-AKT can be made by immunization of mice with each of the above KLH conjugates in CFA, then fusion of spleen cells with Sp2/0, followed by HAT selective medium cultivation, screening and cloning of resultant antibody-producing hybridomas. Antibodies are purified by DEAE ion exchange chromatography, Sephadex gel filtration, and affinity chromatography.

### Example 23

Hybridization kits are described for the detection of Bmpr1a Wt and Bmpr1a variant nucleic acid sequences. Bmpr1a Wt and variant nucleic acid sequence molecules are prepared by either PCR methodology, including real time PCR techniques, or conventional cloning technology as is known in the art. Probe nucleic acid sequences can be produced in vectors as previously described. As alternatives to PCR methodology, isothermal techniques (Guatelli et al., Proceeding of the National Academy of Science 87: 1874-1878 (1990)), transcription based methods (Kwoh et al., Proceedings National Academy of Science 86: 1173-1177 (1989)), and QB replicase techniques (Munishkin et al., Nature 33: 473 (1988)) may be used. DNA or RNA primers are prepared containing desired Bmpr1a probe sequences. For example, a nucleic acid probe can be prepared to different portions of Bmpr1a nucleic acid sequences. Similarly, probes can be prepared for nucleic acid sequences that encode inactive Bmpr1a polypeptide variants that either do not bind to LRP5 or LRP6 or, alternatively, that, when inserted into mammalian cells, cause phenotypic characteristic changes manifested as increased ISC number, increased self-renewal, proliferation, and/or polyposis.

Bmpr1a Wt molecule and Bmpr1a variant cDNA synthesis and DIG labeling can be performed as follows: 10-15 μg Bmpr1a sample RNA is heated with 1.7 μl random primers (3 μg/μl; Invitrogen Cat. No. 48190-011) and 15.9 μl H₂O at 70° C. The mixture is snap cooled on ice and centrifuge. To each reaction tube, DIG-dCTP is added. The master mix is made by adding Strand Buffer, DTT, dNTPs (25 mM each dA/G/TTP, 10 mM dCTP) and SuperScript II (200 U/μl; Invitrogen Cat. No. 18064-014). Then, the reaction is incubated at 25° C., followed by 42° C. incubation.

Using the MinElute PCR purification kit (Qiagen Cat. No. 28004), DIG-labeled cDNA samples are applied to a MinElute column, then centrifuged. For hybridization, cDNA is denatured and exposed to hybridization solution in a pre-heated hybridization chamber. An optional label attached to the nucleic acid can be a radioactive, colorimetric, enzymatic, or fluourometric label, as is known in the art. After incubation, hybridization slides are washed and scanned using the ScanArray Express (Perkin Elmer Life Sciences, Boston, Mass.). Alternatively, the Image Trak Epi-fluorescence System (Perkin Elmer Life Sciences, Boston, Mass.) can be used for 96, 384, or 1536 well plates.

All references cited in the preceding text of the patent application or in the following reference list, to the extent that they provide exemplary, procedural, or other details supplementary to those set forth herein, are specifically incorporated by reference to the same extent as if each individual publication or patent application was specifically and individually indicated to be incorporated by reference.

Thus, there has been shown and described an invention for supporting intestinal stem cell proliferation, self-renewal, and differentiation which fulfills all the objects and advantages therefor. It is apparent to those of skill in the art, however, that many changes, variations, modifications, and other uses and applications to the invention are possible, and also such changes, variations, modifications, and other uses and applications which do not depart from the spirit and scope of the invention are deemed to be covered by the invention, which is limited only by the claims which follow.

### REFERENCES

Altschul et al. (J. Mol. Biol. 215:403-410. (1990)).
Altschul et al. (Nucleic Acids Res. 25:3389-3402. (1990)).
Bei, M. and Maas, R. FGFs and BMP4 induce both Msx1-independent and Msx1-dependent signaling pathways in early tooth development. Development 125, 4325-4333 (1998).
Belletti, B., Prisco, M., Morrione, A., Valentinis, B., Navarro, M., and Baserga, R. Regulation of Id2 gene expression by the insulin-like growth factor I receptor requires signaling by phosphatidylinositol 3-kinase. J Biol Chem 276, 13867-13874 (2001).
Bitgood, M. J., and McMahon, A. P. Hedgehog and Bmp genes are coexpressed at many diverse sites of cell-cell interaction in the mouse embryo. Dev Biol 172, 126-138 (1995).
Booth, C., and Potten, C. S. Gut instincts: thoughts on intestinal epithelial stem cells. J Clin Invest 105, 1493-1499 (2000).
Brittan, M., and Wright, N. A. Gastrointestinal stem cells. J Pathol 197, 492-509 (2002).
Clement, J. H., Marr, N., Meissner, A., Schwalbe, M., Sebald, W., Kliche, K. O., Hoffken, K., and Wolfl, S. Bone morphogenetic protein 2 (BMP-2) induces sequential changes of Id gene expression in the breast cancer cell line MCF-7. J Cancer Res Clin Oncol 126, 271-279 (2000).
Cotran, R., Kumar, v. and Collins, T. Small and large intestines. Sixth Edition of “Robinson Pathological Basis of Dieases” by W B Saunders Company Chapter 18, 828 (1999).
Cotsarelis, G., Sun, T. T., and Lavker, R. M. Label-retaining cells reside in the bulge area of pilosebaceous unit: implications for follicular stem cells, hair cycle, and skin carcinogenesis. Cell 61, 1329-1337 (1990).
Dale, L. & Wardle, F. C. A gradient of BMP activity specifies dorsal-ventral fates in early Xenopus embryos. Semin Cell Dev Biol 10, 319-26 (1999).
Datta, S. R. et al. AKT phosphorylation of BAD couples survival signals to the cell-intrinsic death machinery. Cell 91, 231-41 (1997).
Deng, W. & Lin, H. Spectrosomes and fusomes anchor mitotic spindles during asymmetric germ cell divisions and facilitate the formation of a polarized microtubule array for oocyte specification in Drosophila. Dev Biol 189, 79-94 (1997).
Entchev, E. V., Schwabedissen, A. & Gonzalez-Gaitan, M. Gradient formation of the TGF-beta homolog Dpp. Cell 103, 981-91 (2000).
Fero, M. L. et al. A syndrome of multiorgan hyperplasia with features of gigantism, tumorigenesis, and female sterility in p27(Kip1)-deficient mice. Cell 85, 733-44 (1996).
Fuchs, E., Merrill, B. J., Jamora, C., and DasGupta, R. At the roots of a never-ending cycle. Dev Cell 1, 13-25 (2001).
Fukumoto, S., Hsieh, C. M., Maemura, K., Layne, M. D., Yet, S. F., Lee, K. H., Matsui, T., Rosenzweig, A., Taylor, W. G., Rubin, J. S., et al. AKT participation in the Wnt signaling pathway through Dishevelled. J Biol Chem 276, 17479-17483 (2001).
Ghosh-Choudhury, N., Abboud, S. L., Nishimura, R., Celeste, A., Mahimainathan, L., and Choudhury, G. G. Requirement of BMP-2-induced phosphatidylinositol 3-kinase and AKT serine/threonine kinase in osteoblast differentiation and Smad-dependent BMP-2 gene transcription. J Biol Chem 277, 33361-33368 (2002).
Groszer, M. et al. Negative regulation of neural stem/progenitor cell proliferation by the PTEN tumor suppressor gene in vivo. Science 294, 2186-9 (2001).
Guatelli et al., Proceeding of the National Academy of Science 87: 1874-1878 (1990)).
Harlow, E and Lane, D. Antibodies: A Laboratory Manual, Cold Spring Harbor Laboratory, New York, 1988.
Hefferan, T. E. et al. Overexpression of a nuclear protein, TIEG, mimics transforming growth factor-beta action in human osteoblast cells. J Biol Chem 275, 20255-9 (2000).
Hermiston, M. L. & Gordon, J. I. Organization of the crypt-villus axis and evolution of its stem cell hierarchy during intestinal development. Am J Physiol 268, G813-22 (1995).
Hogan, B. L. Bone morphogenetic proteins in development. Curr Opin Genet Dev 6, 432-8 (1996).
Houlston, R., Bevan, S., Williams, A., Young, J., Dunlop, M., Rozen, P., Eng, C., Markie, D., Woodford-Richens, K., Rodriguez-Bigas, M. A., et al. Mutations in DPC4 (SMAD4) cause juvenile polyposis syndrome, but only account for a minority of cases. Hum Mol Genet 7, 1907-1912 (1998).
Howe, J. R., Bair, J. L., Sayed, M. G., Anderson, M. E., Mitros, F. A., Petersen, G. M., Velculescu, V. E., Traverso, G., and Vogelstein, B. Germline mutations of the gene encoding bone morphogenetic protein receptor 1A in juvenile polyposis. Nat Genet 28, 184-187 (2001).
Howe, J. R., Roth, S., Ringold, J. C., Summers, R. W., Jarvinen, H. J., Sistonen, P., Tomlinson, I. P., Houlston, R. S., Bevan, S., Mitros, F. A., et al. Mutations in the SMAD4/DPC4 gene in juvenile polyposis. Science 280, 1086-1088 (1998).
Hu, G., Lee, H., Price, S. M., Shen, M. M., and Abate-Shen, C. Msx homeobox genes inhibit differentiation through upregulation of cyclin D1. Development 128, 2373-2384 (2001).
Hurlstone, A., and Clevers, H. T-cell factors: turn-ons and turn-offs. Embo J 21, 2303-2311 (2002).
Itoh, H. et al. Activation of immediate early gene, c-fos, and c-jun in the rat small intestine after ischemia/reperfusion. Transplantation 69, 598-604 (2000).
Itoh, S., Itoh, F., Goumans, M. J. & Ten Dijke, P. Signaling of transforming growth factor-beta family members through Smad proteins. Eur J Biochem 267, 6954-67 (2000).
Kandel, E. S. & Hay, N. The regulation and activities of the multifunctional serine/threonine kinase AKT/PKB. Exp Cell Res 253, 210-29 (1999).
Kang, S. S., Kwon, T., Kwon, D. Y. & Do, S. I. AKT protein kinase enhances human Telomerase activity through phosphorylation of Telomerase reverse transcriptase subunit. J Biol Chem 274, 13085-90 (1999).
Karlin and Altschul. (Proc. Natl. Acad. Sci., USA 87:2264-2268. (1993)).
Khwaja, A., Rodriguez-Viciana, P., Wennstrom, S., Warne, P. H., and Downward, J. Matrix adhesion and Ras transformation both activate a phosphoinositide 3-OH kinase and protein kinase B/AKT cellular survival pathway. Embo J 16, 2783-2793 (1997).
Kimura, T. et al. Conditional loss of PTEN leads to testicular teratoma and enhances embryonic germ cell production. Development 130, 1691-1700 (2003).
King, K. E., Iyemere, V. P., Weissberg, P. L. & Shanahan, C. M. Kruppel-like factor 4 (KLF4/GKLF) is a target of bone morphogenetic proteins and transforming growth factor beta 1 in the regulation of vascular smooth muscle cell phenotype. J Biol Chem 278, 11661-9 (2003).
Kobayashi, T., Niimi, S., Fukuoka, M. & Hayakawa, T. Regulation of inhibin beta chains and follistatin mRNA levels during rat hepatocyte growth induced by the peroxisome proliferator di-n-butyl phthalate. Biol Pharm Bull 25, 1214-6 (2002).
Korinek, V., Barker, N., Moerer, P., van Donselaar, E., Huls, G., Peters, P. J., and Clevers, H. Depletion of epithelial stem-cell compartments in the small intestine of mice lacking Tcf-4. Nat Genet 19, 379-383 1998).
Kuhn, R., Schwenk, F., Aguet, M. & Rajewsky, K. Inducible gene targeting in mice. Science 269, 1427-9 (1995).
Kwoh et al., Proceedings National Academy of Science 86: 1173-1177 (1989)).
Lim, D. A., Tramontin, A. D., Trevejo, J. M., Herrera, D. G., Garcia-Verdugo, J. M., and Alvarez-Buylla, A. Noggin antagonizes BMP signaling to create a niche for adult neurogenesis. Neuron 28, 713-726 (2000).
Luongo, C., Moser, A. R., Gledhill, S., and Dove, W. F. Loss of Apc+ in intestinal adenomas from Min mice. Cancer Res 54, 5947-5952 (1994).
Maehama, T. & Dixon, J. E. The tumor suppressor, PTEN/MMAC1, dephosphorylates the lipid second messenger, phosphatidylinositol 3,4,5-trisphosphate. J Biol Chem 273, 13375-8 (1998).
Maggio, E. T. Ed., Enzyme-Immunoassay, CRC Press, Florida, 1980.
Massague, J. TGF-beta signal transduction. Annu Rev Biochem 67, 753-91 (1998).
McMahon, J. A., Takada, S., Zimmerman, L. B., Fan, C. M., Harland, R. M., and McMahon, A. P. Noggin-mediated antagonism of BMP signaling is required for growth and patterning of the neural tube and somite. Genes Dev 12, 1438-1452 (1998).
Mishina, Y., Hanks, M. C., Miura, S., Tallquist, M. D., and Behringer, R. R. Generation of Bmpr/Alk3 conditional knockout mice. Genesis 32, 69-72 (2002).
Mishina, Y., Suzuki, A., Ueno, N., and Behringer, R. R. Bmpr encodes a type I bone morphogenetic protein receptor that is essential for gastrulation during mouse embryogenesis. Genes Dev 9, 3027-3037 (1995).
Morrison, S. J., Prowse, K. R., Ho, P. & Weissman, I. L. Telomerase activity in hematopoietic cells is associated with self-renewal potential. Immunity 5, 207-16. (1996).
Munishkin et al., Nature 33: 473 (1988)).
Nagasako, T., Sugiyama, T., Mizushima, T., Miura, Y., Kato, M., and Asaka, M. Up-regulated Smad5 Mediates Apoptosis of Gastric Epithelial Cells Induced by Helicobacter pylori Infection. J Biol Chem 278, 4821-4825 (2003).
Nakamura, Y., Ozaki, T., Koseki, H., Nakagawara, A. & Sakiyama, S. Accumulation of p27 KIP1 is associated with BMP2-induced growth arrest and neuronal differentiation of human neuroblastoma-derived cell lines. Biochem Biophys Res Commun 307, 206-13 (2003).
Novak, A., Guo, C., Yang, W., Nagy, A., and Lobe, C. G. Z/EG, a double reporter mouse line that expresses enhanced green fluorescent protein upon Cre-mediated excision. Genesis 28, 147-155 (2000).
Penninger, J. M. & Woodgett, J. Stem cells. PTEN—coupling tumor suppression to stem cells? Science 294, 2116-8 (2001).
Polakis, P. Wnt signaling and cancer. Genes Dev 14, 1837-1851 (2000).
Potten, C. S., and Booth, C. Keratinocyte stem cells: a commentary. J Invest Dermatol 119, 888-899 (2002).
Powell, S. M., Zilz, N., Beazer-Barclay, Y., Bryan, T. M., Hamilton, S. R., Thibodeau, S. N., Vogelstein, B., and Kinzler, K. W. APC mutations occur early during colorectal tumorigenesis. Nature 359, 235-237 (1992).
Prendergast, G. C. & Ziff, E. B. A new bind for Myc. Trends Genet 8, 91-6 (1992).
Quaroni, A., Tian, J. Q., Seth, P. & Ap Rhys, C. p27(Kip1) is an inducer of intestinal epithelial cell differentiation. Am J Physiol Cell Physiol 279, C1045-57 (2000).
Ray, P. et al. Inducible expression of keratinocyte growth factor (KGF) in mice inhibits lung epithelial cell death induced by hyperoxia. Proc Natl Acad Sci USA 100, 6098-103 (2003).
Roberts, D. J. Molecular mechanisms of development of the gastrointestinal tract. Dev Dyn 219, 109-120 (2000).
Spradling, A., Drummond-Barbosa, D. & Kai, T. Stem cells find their niche. Nature 414, 98-104 (2001).
Teleman, A. A., and Cohen, S. M. Dpp gradient formation in the Drosophila wing imaginal disc. Cell 103, 971-980 (2000).
Travers, A. A. Priming the nucleosome: a role for HMGB proteins? EMBO Rep 4, 131-6 (2003).
Vazquez, F., Ramaswamy, S., Nakamura, N., and Sellers, W. R. Phosphorylation of the PTEN tail regulates protein stability and function. Mol Cell Biol 20, 5010-5018 (2000).
Waite, K. A. & Eng, C. BMP2 exposure results in decreased PTEN protein degradation and increased PTEN levels. Hum Mol Genet 12, 679-684 (2003).
Weissman, I. L. Developmental switches in the immune system. Cell 76, 207-218 (1994).
Wice, B. M., and Gordon, J. I. Forced expression of Id-1 in the adult mouse small intestinal epithelium is associated with development of adenomas. J Biol Chem 273, 25310-25319 (1998).
Wilson, J. W., Deed, R. W., Inoue, T., Balzi, M., Becciolini, A., Faraoni, P., Potten, C. S., and Norton, J. D. Expression of Id helix-loop-helix proteins in colorectal adenocarcinoma correlates with p53 expression and mitotic index. Cancer Res 61, 8803-8810 (2001).
Wong, G. A., Tang, V., El-Sabeawy, F. & Weiss, R. H. BMP-2 inhibits proliferation of human aortic smooth muscle cells via p21Cip1/Waf1. Am J Physiol Endocrinol Metab 284, E972-9 (2003).
Wu, H., Goel, V. & Haluska, F. G. PTEN signaling pathways in melanoma. Oncogene 22, 3113-22 (2003).
Yamashita, Y. M., Jones, D. L. & Fuller, M. T. Orientation of asymmetric stem cell division by the APC tumor suppressor and centrosome. Science 301, 1547-50 (2003).
Yi, S. E., Daluiski, A., Pederson, R., Rosen, V. & Lyons, K. M. The type I BMP receptor BMPRIB is required for chondrogenesis in the mouse limb. Development 127, 621-30 (2000).
Yi, S. E., Daluiski, A., Pederson, R., Rosen, V., and Lyons, K. M. The type I BMP receptor BMPRIB is required for chondrogenesis in the mouse limb. Development 127, 621-630 (2000).
Zhang, J. et al. Dissection of promoter control modules that direct BMP4 expression in the epithelium-derived components of hair follicles. Biochem Biophys Res Commun 293, 1412-9 (2002).
Zhang, J., Niu, C., Feng, J., He, X., Johnson, T., Haug, J., Harris, S., Wiedemann, L., Mishina, Y. and Li, L. Identification of the Hematopoietic Stem Cell Niche and Control of the Niche Size. Nature In press (2003).

What is claimed is:

1. A vector for use in transfecting an embryonic stem cell, whereby clonal changes in adult intestinal tissue can be promoted by a recombination activator, comprising:
(a) at least two conditional recombination sites; and,
(b) a Bmpr1a nucleotide sequence located between the sites, whereby the vector inserts the recombination sites and transgenic nucleotide sequence into a Bmpr1a sequence of the embryonic stem cell.

2. The vector of claim 1, wherein the vector is selected from the group consisting of expression vectors, fusion vectors, gene therapy vectors, two-hybrid vectors, reverse two-hybrid vectors, sequencing vectors, expression kits, and cloning vectors.

3. The vector of claim 1, wherein the recombination sites are LoxP.

4. The vector of claim 1, wherein the vector is selected from the group consisting of eukaryotic and prokaryotic vectors.

5. The eukaryotic vector of claim 4, wherein the vector is selected from the group consisting of MSCV, Harvey murine sarcoma virus, pFastBac, pFastBac HT, pFastBac DUAL, pSFV, pTet-Splice, pEUK-C1, pPUR, pMAM, pMAMneo, pBI101, pBI121, pDR2, pCMVEBNA, YACneo, pSVK3, pSVL, pMSG, pCH110, pKK232-8, p3′SS, pBlueBacIII, pCDM8, pcDNA1, pZeoSV, pcDNA3, pREP4, pCEP4, and pEBVHis vectors.

6. The prokaryotic vector of claim 4, wherein the vector is selected from the group consisting of pET, pET28, pcDNA3.11V5-His-TOPO, pCS2+, pcDNA II, pSL301, pSE280, pSE380, pSE420, pTrcHis, pRSET, pGEMEX-1, pGEMEX-2, pTrc99A, pKK223-3, pGEX, pEZZ18, pRIT2T, pMC1871, pKK233-2, pKK38801, and pProEx-HT.

7. The vector of claim 1, wherein the Bmpr1a nucleotide sequence is selected from the group consisting of Bmpr1a homologs, degenerate variants, mutants, orthologs, Wt sequences, fragments, and related nucleotide sequences.

8. The vector of claim 1, wherein the Bmpr1a nucleotide sequence is selected from the group consisting of SEQ ID NOs 1, 2, 3, 6, and 8.

9. The vector of claim 1, wherein the Bmpr1a nucleotide sequence is selected from the group consisting of any nucleotide sequence homologous to the Bmpr1a nucleotide sequence or a fragment of the Bmpr1a nucleotide sequence.

10. A vector for use in transfecting an embryonic stem cell, comprising:
(a) at least one conditional recombination site; and,
(b) a BMP nucleotide sequence.

11. A vector for use in transfecting an embryonic stem cell, comprising:
(a) A nucleotide sequence selected from the group consisting of SEQ ID NO 1, 2, 3, 6, and 8; and,
(b) two LoxP sites flanking the nucleotide sequence, whereby the vector can be used to transfect an embryonic stem cell to produce Bmpr1a^{fx/fx} progeny.

12. A vector for producing a conditionally activated mutant, comprising:
(a) a Bmpr1a nucleotide sequence; and,
(b) two recombination sites.

13. A vector for transforming cells in a differentiated intestinal cell, comprising:
(a) a Bmpr1a nucleotide sequence; and,
(b) a vector.

14. An embryonic stem cell transfected by the vector of claim 1.

15. The vector of claim 1, comprising a selectable marker selected from the group consisting of LacZ, neo, Fc, DIG, Myc, and FLAG.

16. An embryonic stem cell comprising a transgenic Bmpr1a sequence flanked by recombination sites.

17. An embryonic stem cell transfected with a transgenic conditional mutant sequence.

18. A conditional mutant intestinal stem cell comprising:
(a) a transgenic nucleotide sequence selected from the group consisting of BMP and Bmpr1a; and,
(b) at least two recombination sites flanking the nucleotide sequence.

19. The cell of claim 18, wherein the cell is selected from the group consisting of in vivo and in vitro cells.

20. The cell of claim 18, wherein the cell is mammalian.

21. The cell of claim 20, wherein the mammalian cell is selected from the group consisting of mice, rat, primate, and human cells.

22. The cell of claim 18, wherein it is contacted with a recombination activator to produce a mutant intestinal stem cell.

23. An intestinal cell, comprising a floxed Bmpr1a nucleotide sequence, wherein the intestinal cell is a conditional knock-out.

24. The intestinal cell of claim 23, wherein the intestinal cell is selected from the group consisting of in vivo transfected cells and in vitro transfected cells.

25. The intestinal cell of claim 23, wherein the cell is selected from the group consisting of intestinal stem, transient amplifying progenitor, paneth, goblet, enterocytes, mucosal progenitor, endocrine and columnar progenitor cells.

26. The intestinal cell of claim 23, wherein the cell is derived from tissue selected from the group consisting of stomach, intestine, digestive tract, duodenum, and colon cells.

27. The intestinal cell of claim 23, wherein the cell is contacted with a recombination activator to form a mutant intestinal cell.

28. A mutant intestinal stem cell comprising a Bmpr1a mutant selected from the group consisting of frame shift, point substitution, loss of function, knock-out deletion, and conventional deletion mutations.

29. An intestinal stem cell comprising an inactive BMP, wherein BMP protein binding to Bmpr1a is inhibited.

30. The intestinal stem cell of claim 29, wherein the cell is selected from the group consisting of in vivo and in vitro cells.

31. An intestinal stem cell comprising an inactive, truncated Bmpr1a receptor polypeptide formed by a conditional mutant.

32. The intestinal stem cell of claim 31, wherein the cell is an ISC having increased nuclear accumulation of β-catenin and P-PTEN.

33. An intestinal stem cell having increased self-renewal capacity and having increased P-PTEN, AKTS473, nuclear β-catenin, 14-3-3ζ, and Tert proteins associated with the cell.

34. An intestinal stem cell, wherein a Bmpr1a nucleotide sequence is knocked out.

35. A mutant intestinal stem cell, comprising an inactive BMP, wherein the cells are selected from the group consisting of in vivo and in vitro cells, and the cells are selected from the group consisting of intestinal stem, transient amplifying progenitor, paneth, goblet, enterocytes, mucosal progenitor, and columnar progenitor cells.

36. An intestinal cell population selected from the group consisting of intestinal stem, transient amplifying progenitor, paneth, goblet, enterocytes, mucosal progenitor, and columnar progenitor cells, wherein BMP is inhibited from binding to Bmpr1a sequences in the cells.

37. In vivo intestinal tissue comprising mutant clonal cells located in crypt and villus regions with the cells formed from a transgenic Bmpr1a nucleotide sequence.

38. The tissue of claim 37, wherein intestinal stem cells divide symmetrically and asymmetrically.

39. The tissue of claim 37, having increased populations of paneth and goblet cells.

40. The tissue of claim 37, wherein crypt fission has occurred.

41. The tissue of claim 37, having a reduced population of columnar progenitor cells.

42. The tissue of claim 37, having multiple polyps.

43. The tissue of claim 37, having reduced apoptosis.

44. The tissue of claim 37, having increased P-BAD and 14-3-3ζ.

45. The tissue of claim 37, having increased P-Smad1,5,8.

46. In vitro intestinal tissue comprising Bmpr1a mutant clonal cells located in crypt and villus regions.

47. The tissue of claim 46, wherein intestinal stem cells divide symmetrically and asymmetrically.

48. The tissue of claim 46, having increased populations of paneth and goblet cells.

49. The tissue of claim 46, wherein crypt fission has occurred.

50. The tissue of claim 46, having a reduced population of columnar progenitor cells.

51. The tissue of claim 46, having reduced apoptosis.

52. The tissue of claim 46, having impaired epithelial differentiating and unbalanced lineage commitment.

53. In vivo intestinal tissue, comprising:
(a) mutant intestinal stem cell, whereby Bmpr1a has been knocked-out to block BMP binding;
(b) abnormally differentiated mucosal progenitor cells;
(c) fused crypts; and,
(d) increased intestinal stem cell proliferation.

54. A nucleotide sequence comprising Bmpr1a flanked by at least two recombination sites.

55. The nucleotide sequence of claim 54, wherein at least two recombination sites are conditional recombination sites.

56. SEQ ID NO 1 flanked by LoxP.

57. A mutant Mx1-Cre⁺Bmpr1a^{fx/fx} organism comprising a mutant intestinal cell, wherein an inactivated Bmpr1a cell receptor polypeptide is expressed.

58. The mutant Mx1-Cre⁺Bmpr1a^{fx/fx} organism of claim 57, wherein the mutant intestinal cell is selected from the group consisting of intestinal epithelial, intestinal stem, transient amplifying progenitor, mucosal progenitor, columnar progenitor, enterocyte, mesenchymal, paneth, goblet, and enteroendocrine cells.

59. A mutant Bmpr1a organism having a mutant intestinal cell comprising a nonfunctional mutant Bmpr1a gene, wherein the gene encodes an inactive Bmpr1a receptor.

60. A post-excision Mx1-Cre⁺Bmpr1a^{fx/fx} knock-out organism having a mutant intestinal cell, wherein a Bmpr1a receptor has been substantially eliminated.

61. A Bmpr1a^{fx/fx} mouse line.

62. An Mx1-Cre⁺Bmpr1a^{fx/fx} mouse.

63. An Mx1-Cre⁺Bmpr1a^{fx/fx} Z/EG mouse.

64. The Mx1-Cre⁺Bmpr1a^{fx/fx} knock-out organism of claim 57, wherein the organism expresses a phenotype selected from the group consisting of expanded ISC number, intestinal polyps, and intestinal tumor phenotypes.

65. An Mx1-Cre⁺Bmpr1a/fx knock-out organism, wherein the mutant intestinal cells express polypeptides selected from the group consisting of inactive and truncated Bmpr1a receptor polypeptides.

66. A pre-excision Bmpr1a^{fx/fx} knock-out mutant organism, comprising intestinal cells having recombination site-flanked Bmpr1a genes.

67. A mutant mouse comprising:
(a) a clonal population of intestinal cells, whereby Bmpr1a is knocked out; and,
(b) an increased population of the intestinal cells in intestinal crypt.

68. A mutant mouse comprising:
(a) a clonal population of intestinal cells whereby Bmpr1a is knocked out; and,
(b) apoptosis in lumen is decreased.

69. A mutant mouse comprising:
(a) a clonal population of intestinal cells whereby Bmpr1a is knocked out; and,
(b) a population of abnormal columnar and mucosal progenitors cells.

70. An in vitro intestinal stem cell cultivation system, comprising:
(a) isolated intestinal tissue, wherein the tissue includes cells that are clonal Bmpr1a knock-out mutants; and,
(b) a culture medium.

71. The stem cell system of claim 70, wherein the clonal mutants are conditional.

72. The stem cell system of claim 70, wherein the mutant is activated and BMP binding to Bmpr1a is inhibited.

73. An in vitro intestinal stem cell cultivation system, comprising:
(a) an isolated intestinal tissue;
(b) a culture medium; and,
(c) at least one stem cell regulator selected from the group consisting of BMP, Noggin, and Ly294002, added in an amount greater than what is found in a Wt tissue.

74. An in vitro intestinal stem cell cultivation system, wherein an intestinal stem cell population proliferates, comprising:
(a) an isolated intestinal stem cell population comprising at least 10⁴ cells;
(b) a culture medium; and,
(c) isolated Noggin polypeptides, wherein Bmpr1a receptor binding to BMP polypeptide is substantially inhibited.

75. An in vitro mutant intestinal Bmpr1a stem cell cultivation system, wherein a mutant intestinal stem cell population proliferates, comprising:
(a) an isolated mutant intestinal Bmpr1a stem cell population comprising at least 10⁴ cells, wherein the cells comprise inactive Bmpr1a cell receptors; and,
(b) a culture medium.

76. An in vitro intestinal stem cell cultivation system for expansion of an intestinal stem cell population comprising:
(a) an isolated intestinal stem cell population comprising at least 10⁴ cells;
(b) an isolated intestinal stem cell activator, wherein the activator is selected from the group consisting of anti-Bmpr1a antibodies, anti-BMP antibodies, Wt Bmpr1a receptor antisense sequences, and fragments thereof; and,
(c) a culture medium.

77. The in vitro intestinal stem cell cultivation system of claim 76, comprising a cell population selected from group consisting of feeder and mesenchymal cell populations.

78. An in vitro intestinal stem cell cultivation system comprising:
(a) an isolated intestinal stem cell population comprising at least 10⁴ cells;
(b) Bmpr1a antisense oligonucleotides, wherein the Bmpr1a antisense oligonucleotides hybridize with Bmpr1a mRNA sequences in cells of the intestinal stem cell population to inhibit Bmpr1a mRNA translation; and,
(c) a culture medium.

79. An in vitro intestinal cell cultivation system comprising:
(a) isolated intestinal tissue;
(b) a culture medium; and
(c) an activator selected from the group consisting of BMP, Noggin, and Ly294002, added in an amount greater than what is found in a Wt tissue.

80. A method for forming a pre-excision conditional Mx1-Cre-Lox Bmpr1a^{fx/fx} knock-out mutant organism, comprising:
(a) isolating a Bmpr1a gene;
(b) forming a modified Bmpr1a gene, wherein the modified Bmpr1 gene is flanked by Lox recombination sites and has a markers;
(c) forming a Bmpr1a vector by insertion of the modified Bmpr1a gene into a vector;
(d) transfecting an embryonic stem cell with the Bmpr1a vector to form a Bmpr1a embryonic stem cell;
(e) inserting the Bmpr1a embryonic stem cell into a host uterus, wherein a Bmpr1a^{fx/fx} organism is formed; and,
(f) crossing the Bmpr1a^{fx/fx} organism with an Mx1-Cre organism to produce Mx1-Cre-Lox Bmpr1a^{fx/fx} progeny.

81. The method of claim 80, wherein Bmpr1a vector formation comprises inserting marker sites into the vector's genomic sequence.

82. The method of claim 80, wherein Bmpr1a vector formation comprises inserting at least one of LacZ and GFP marker sites into the vector's genomic sequence.

83. A method for making a post-excision Mx1-Cre⁺Bmpr1a^{fx/fx} knock-out mutant organism for use in studying an intestinal cell population comprising:
(a) making the hybrid pre-excision Mx1-Cre-Lox Bmpr1a^{fx/fx} knock-out mutant organism by the method of claim 80; and,
(b) administering a recombination activator to the hybrid pre-excision Mx1-Cre Bmpr1a^{fx/fx} knock-out mutant organism, wherein Cre-mediated Lox site-directed Bmpr1a gene recombination is induced to yield substantially eliminated Bmpr1a intestinal cell receptor genes.

84. The method of claim 80, comprising administering Poly I:C at P2 or P20.

85. A method for generating a mutant phenotypic change in an intestinal tissue in vivo, wherein the phenotypic change is selected from the group consisting of expanded intestinal stem cell population, increased self-renewal activity, differentiation change, reduced apoptosis, crypt fission, symmetrical intestinal stem cell division, and polyposis, comprising:
(a) isolating a Bmpr1a gene in a Wt Bmpr1a organism;
(b) forming a modified Bmpr1a gene, wherein the modified Bmpr1 gene comprises Lox recombination sites flanking the Bmpr1a gene and a marker;
(c) forming a Bmpr1a vector by insertion of the modified Bmpr1a gene into a vector;
(d) transfecting an embryonic stem cell with the Bmpr1a vector to form a Bmpr1a embryonic stem cell;
(e) inserting the Bmpr1a embryonic stem cell into a host uterus, wherein a Bmpr1a^{fx/fx} organism is formed;
(f) crossing the Bmpr1a^{fx/fx} organism with an Mx1-Cre organism to form a hybrid Mx1-Cre-Lox Bmpr1a^{fx/fx} organism; and,
(g) injecting a recombination activator into the hybrid Mx1-Cre-Lox Bmpr1a^{fx/fx} embryo, wherein recombination results in expression of inactive Bmpr1a cell receptors.

86. The method of claim 85, wherein the recombination activator injection is performed at a postnatal time selected from the group consisting of 1, 2, and 20 days.

87. A method for forming a post-excision Mx1-Cre⁺Bmpr1a^{fx/fx} Z/EG knock-out mutant organism for use in studying an intestinal cell comprising:
(a) making a hybrid pre-excision Mx1-Cre-Lox Bmpr1a^{fx/fx} knock-out mutant organism;
(b) crossing the pre-excision Mx1-Cre-Lox Bmpr1a^{fx/fx} organism with a Z/EG organism, wherein a pre-excision hybrid Mx1-Cre-Lox Bmpr1a^{fx/fx} Z/EG organism is formed; and,
(c) administering a recombination activator to the hybrid Mx1-Cre-Lox Bmpr1a^{fx/fx} Z/EG organism, wherein Cre-mediated Lox site-directed intracellular Bmpr1a gene recombination is induced.

88. A method for increasing an intestinal stem cell population number in vitro comprising:
(a) isolating a Wt intestinal tissue;
(b) exposing the intestinal tissue to an stem cell activator, wherein the activator induces intestinal stem cell proliferation; and,
(c) cultivating the intestinal tissue in culture medium in vitro.

89. The method of claim 88, wherein the activator is Noggin.

90. The method of claim 88, wherein the Noggin concentration in medium is between 10 ng/ml and 200 ng/ml.

91. A method for studying effect of a regulator upon intestinal stem cell population in vitro, comprising:
(a) isolating a Wt intestinal tissue;
(b) exposing the intestinal tissue to a stem cell regulator selected from the group consisting of BMP, Noggin, and Ly294002;
(c) cultivating the intestinal tissue in culture medium in vitro; and,
(d) assessing the regulator's effect upon intestinal stem cell population number.

92. The method of claim 91, wherein the exposure of the intestinal tissue to the regulator is selected from the group consisting of injection, bead-mediated transfer, particle-mediated transfer, liposome transfer, transfection, and electroporesis.

93. A method for making a mouse model for human juvenile intestinal polyposis comprising:
(a) forming a pre-excision Bmpr1a mutant Mx1-Cre-Lox mouse pup; and,
(b) administering a recombination activator to excise a Bmpr1a gene to form a post-excision Bmpr1a mutant Mx1-Cre-Lox mouse pup, wherein the Bmpr1a receptor is inactivated.

94. A method for using the post-excision Bmpr1a mutant Mx1-Cre-Lox mouse pup of claim 93 as a mouse model for human juvenile intestinal polyposis comprising: detecting a phenotypic change in murine intestinal tissue selected from the group consisting of polyposis, crypt fission, increased cell proliferation, abnormal differentiation, and reduced apoptosis.

95. The method of claim 93 for using the mouse model for human juvenile intestinal polyposis, comprising detecting at least one marker associated with a cell in the mouse selected from the group consisting of goblet, paneth, mucin-producing, enterocyte, tumorous, and polyp cells.

96. A method for forming a mutant intestinal stem cell population number in vitro comprising:
(a) isolating a Wt intestinal stem cell population comprising at least 10⁴ cells;
(b) forming antibodies selected from the group consisting of anti-Bmpr1a receptor antibodies and anti-BMP antibodies;
(c) isolating the antibodies;
(d) administering the isolated activating antibodies to intestinal stem cells in vitro, wherein the antibodies operatively prevent binding of Bmpr1a receptor polypeptides to BMP polypeptides; and,
(e) cultivating the intestinal stem cell population in vitro in a growth medium.

97. The method of claim 96, wherein the administration of isolated activating antibodies to intestinal stem cells is selected from the group consisting of injection, transfection, micro-vessel encapsulation, particle-mediated delivery, diffusion, and liposome encapsulation.

98. A method for forming a mutant intestinal stem cell population number in vitro comprising:
(a) isolating a Wt intestinal stem cell population comprising at least 10⁴ cells;
(b) forming Bmpr1a antisense oligonucleotides;
(c) isolating the Bmpr1a antisense oligonucleotides;
(d) administering the isolated Bmpr1a antisense oligonucleotides into intestinal stem cells in vitro, wherein the oligonucleotides operably hybridize with Bmpr1a mRNA sequences to prevent intracellular translation of Bmpr1a polypeptides; and,
(e) cultivating the intestinal stem cell population in vitro in a growth medium.

99. The method of claim 98, wherein the administration of the antisense oligonucleotides into the intestinal stem cell population is selected from the group consisting of microinjection, transfection, micro-vessel transfer, particle bombardment, biolistic particle delivery, liposome mediated transfer, and electroporation

100. A kit for detecting marker polypeptides associated with polyposis in cells of an intestinal cell population, wherein the kit comprises:
(a) a container; and,
(b) an anti-marker antibody attached to a label, wherein the anti-marker antibody binds to a marker polypeptide selected from the group consisting of P-PTEN, P-AKT, Tert, 14-3-3ζ, β-catenin, P-BAD, and Ki67.

101. A kit for detecting BMP mutants in an intestinal cell population, wherein the kit comprises:
(a) a container;
(b) at least two marker nucleic acid probes attached to a label, wherein the marker nucleic acid probes are selected from the group consisting of BMP, Noggin, PTEN, P-PTEN, AKT, P-AKT, Tert, β-catenin, Ki67, p27, Smad1,5,8, tubulin, Chromgrin A, BAD, PBAD, and FAK nucleic acid sequence probes; and,
(c) control Wt intestinal cell population.

102. A method for detecting a marker polypeptide in target cells of an intestinal cell population comprising:
(a) immunizing an animal with a marker selected from the group consisting of Bmpr1a, BMP, Noggin, PTEN, P-PTEN, AKT, PAKT, Tert, β-catenin, Ki67, p27, Smad1,5,8, tubulin, Chromgrin A, BAD, PBAD, and FAK polypeptides, and mutant polypeptides thereof;
(b) isolating the marker antibody, wherein the marker antibody binds to the marker;
(c) attaching a label to the isolated marker antibody to form a labeled anti-marker antibody;
(d) administering the labeled anti-marker antibody to a target cell of the intestinal cell population in an intestinal cell preparation; wherein the labeled anti-marker antibody binds to a marker polypeptide in the target cell; and,
(e) detecting the presence of the labeled anti-marker antibody in the target cell, wherein the labeled antibody identifies the presence of the marker polypeptide in the target cell.

103. A method for detecting a marker nucleic acid in target cells of an intestinal cell population, comprising:
(a) forming a marker nucleic acid probe selected from the group consisting of BMP, Noggin, PTEN, P-PTEN, AKT, PAKT, Tert, β-catenin, Ki67, p27, Smad1,5,8, tubulin, Chromgrin A, BAD, PBAD, and FAK nucleic acid sequence probes, and mutant probes thereof;
(b) amplifying the marker nucleic acid probe;
(c) attaching a label to the marker nucleic acid probe to form labeled marker nucleic acid probe;
(d) administering the labeled marker nucleic acid probe to a target cell of the intestinal cell population; and,
(e) detecting the label in the target cell, wherein the label identifies the presence of the marker nucleic acid probe in the target cell.

104. A kit for detecting mutant BMP pathway signaling in an intestinal tissue, wherein the kit comprises:
(a) a container;
(b) a mutant Wt intestinal tissue; and,
(c) at least two labeled antibodies selected from the group consisting of antibodies to PTEN, P-PTEN, AKT, activated AKT, β-catenin, Tert, α-tubulin, γ-tubulin, FAK, BAD, and P-BAD.

105. The kit of claim 104, comprising a control Wt intestinal tissue.

106. The kit of claim 104, wherein the label is selected from the group consisting of fluorescent, phosphorescent, luminescent, radioactive, and chromogenic labels.

107. A kit for detecting mutant BMP pathway signaling in an intestinal cell population, wherein the kit comprises:
(a) a container;
(b) a control Wt intestinal cell population;
(c) BrdU; and,
(d) at least one labeled antibody selected from the group consisting of antibodies to PTEN, P-PTEN, AKT, activated AKT, β-catenin, Tert, α-tubulin, γ-tubulin, FAK, BAD, and P-BAD.

108. A kit for detecting mutant Bmpr1a nucleic acid sequences in intestinal tissue comprising:
(a) a container;
(b) at least one nucleic acid sequence probe, wherein the probe hybridizes to a mutant Bmpr1a sequence region; and,
(c) an intestinal tissue selected from the group consisting of Bmpr1a mutant and Wt tissue.

109. A Western Blot kit for detecting mutant Bmpr1a polypeptide sequences in intestinal tissue comprising:
(a) a container;
(b) Bmpr1a polypeptide standards;
(c) primary antibodies selected from the group consisting of antibodies to Wt Bmpr1a and mutant Bmpr1a polypeptides; and,
(d) labeled secondary antibodies, wherein the binding of labeled secondary antibodies to the primary antibodies permit detection of the mutant Bmpr1a polypeptide sequence in intestinal tissue.

110. A vector comprising a mutant Bmpr1a nucleotide sequence, or fragment thereof, wherein the mutant Bmpr1a sequence encodes an inactive Bmpr1a polypeptide.

111. The vector of claim 110, wherein the mutant Bmpr1a nucleotide sequence is selected from the group consisting of frame shift, deletion, loss of function, point, and substitution mutant sequences.

112. A vector comprising:
(a) a PTEN family nucleotide sequence, wherein the PTEN family is selected from the group consisting of PTEN, AKT, Tert, PI3K, Smad 1,5,8, P27, and mutant genes derived therefrom; and,
(b) at least one recombination site.

113. The vector of claim 112, comprising a promoter.

114. A vector comprising Exon 2 of the Bmpr1a nucleotide sequence.

115. A vector, comprising a PTEN nucleotide sequence, at least one recombination site, and a marker.

116. An intestinal tissue specimen, comprising an intestinal cell population that comprises a mutant PTEN nucleotide sequence.

117. A mutant PTEN organism, comprising a mutant PTEN nucleotide sequence.

118. A mutant mouse, comprising a mutant PTEN nucleotide sequence.

119. An in vitro tissue system comprising:
(a) isolated intestinal tissue; and,
(b) beads possessing a regulator, selected from the group consisting of Noggin, BMP, and Ly294002, wherein the regulator operatively contacts the intestinal tissue.

120. An isolated stem cell population characterized as being Bmrpr1a⁺, Noggin⁺, P-PTEN⁺.

121. An isolated intestinal cell population characterized as being P-PTEN⁺, AKTS473⁺, Tert⁺.

122. An isolated stem cell population characterized as being BMP⁺, PTEN⁺, Smad 1, 5, or 8⁺.

123. The stem cell population of claim 122, wherein the cells are fixed in vitro.

124. An in vivo stem cell population characterized as being P-PTEN⁺, AKTS473⁺, Tert⁺.

125. A group of markers for determining whether intestinal cells are mutagenized, wherein the markers are selected from the group consisting of P-PTEN, PTEN, AKT, P-AKT, Tert, β-catenin, P-Smad1,5,8, BMP, Noggin, Bmpr1a, BAD, P-BAD, 14-3-3ζ, and combinations thereof.

126. Markers for identifying intestinal stem cell self-renewal, comprising AKT and 14-3-3ζ.

127. Markers for identifying stem cell proliferation, comprising BMP, PTEN, P-PTEN, AKT, and P-AKT.

128. Markers for identifying mutant stem cell differentiation β-catenin, P-AKT, P-PTEN, Ki67, and BrdU.

129. Markers for identifying inhibited apoptosis in intestinal cells, comprising BAD, 14-3-3ζ, and TUNEL.

130. An in vitro intestinal tissue sample comprising:
(a) BMP that is blocked from individual stem cells;
(b) an increased number of ISCs self renewing; and,
(c) an increased amount of P-PTEN.

131. An in vitro intestinal tissue sample comprising:
(a) an increased amount of P-PTEN;
(b) an increase in mucosal progenitor cells; and,
(c) a member for causing mutation.

132. The tissue sample of claim 130, wherein the mutation is caused by blocking Bmpr1a or blocking BMP.

133. A pathway which controls self-renewal, proliferation, differentiation, and apoptosis in intestinal tissue, comprising:
(a) a Bmpr1a receptor on an ISC cell surface;
(b) BMP expressed in self-renewal zone;
(c) BMP not expressed in the proliferation zone;
(d) BMP expression progressively increased in the differentiation zone; and,
(e) BMP expressed in the apoptosis zone.

134. A method for preventing apoptosis in intestinal cells comprising blocking BMP binding to a Bmpr1a receptor on a cell selected from the group consisting of paneth, goblet, and enterocyte cells.

135. A method for causing progenitor cells to differentiate into mucosal progenitor cells instead of columnar progenitor cells, comprising blocking BMP binding to Bmpr1a receptors on the progenitor cells.

136. A method for controlling intestinal cell development from self-renewal through apoptosis, comprising preventing binding by BMP to Bmpr1a.

137. A method for controlling proliferation of cells, comprising contacting transient amplifying cells with BMP.

138. A method for causing proliferation of transient amplifying cells comprising blocking BMP with an activator selected from the group consisting of: Noggin, BMP antibodies, and Bmpr1a mutants.

139. A population of ISCs with increased self-renewal identified as P-PTEN⁺, P-AKT⁺, nuclear accumulated β-catenin, 14-3-3 ζ, and Tert⁺.

140. A population of transient amplifying progenitors which are proliferating which are marked Ki67⁺, Brd-U⁺, P-PTEN⁺.

141. A method of regulating β-catenin and Tert comprising controlling BMP which regulates AKT.

142. An isolated group of genes which comprise a pathway for controlling self-renewal, differentiation, and apoptosis in intestinal cells, consisting of: BMP, Noggin, Bmpr1a, PTEN, AKT, Smad1,5,8, β-catenin, and BAD.
